(19) United States
(12) Patent Application Publication
(10) Pub. No.: US 2021/0315869 A1
(43) Pub. Date: Oct. 14, 2021

(54) COMPOUNDS AND METHODS FOR CSK MODULATION AND INDICATIONS THEREFOR

(71) Applicant: Plexxikon Inc., South San Francisco, CA (US)

(72) Inventors: Aaron Albers, San Francisco, CA (US); Zuojun Guo, Pasadena, CA (US); Hiroaki Inagaki, Edogawa-ku (JP); Phuongly Pham, San Francisco, CA (US); Hannah Powers, Oakland, CA (US); Wayne Spevak, Berkeley, CA (US); Jack Walleshauser, Berkeley, CA (US); Jiazhong Zhang, Foster City, CA (US); Ying Zhang, Fremont, CA (US)

(21) Appl. No.: 17/220,665

(22) Filed: Apr. 1, 2021

Related U.S. Application Data
(60) Provisional application No. 63/004,306, filed on Apr. 2, 2020.

(51) Int. Cl. A61K 31/4375; A61P 35/00; C07D 471/04; C07B 59/00; A61K 31/282; A61K 31/675; A61K 31/7068; A61K 31/4745; A61K 31/44; A61K 38/21; A61K 38/20; A61K 31/337; A61K 31/475; A61K 38/14

(52) U.S. Cl. CPC A61K 31/4375; C07B 2200/05; C07D 471/04; C07B 59/002; A61K 31/282; A61K 31/675; A61K 31/7068; A61K 31/4745; A61K 31/44; A61K 38/212; A61K 38/2013; A61K 31/337; A61K 31/475; A61K 38/14; A61P 35/00

(57) ABSTRACT

Disclosed are compounds of Formula (I):

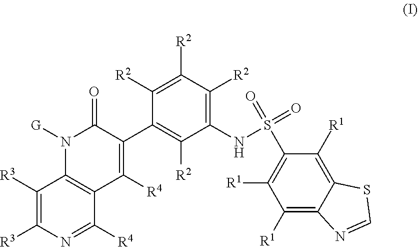

or a pharmaceutically acceptable salt, a tautomer, a stereoisomer, or a deuterated analog thereof, wherein R¹, R², R³, R⁴, and G are as described in any of the embodiments described in this disclosure; compositions thereof; and uses thereof.

### CROSS-REFERENCE TO RELATED APPLICATIONS

The present application claims the benefit under 35 U.S.C. § 119(e) to U.S. Provisional Application No. 63/004,306, filed Apr. 2, 2020, which is hereby incorporated by reference in its entirety.

### FIELD

The present disclosure relates to organic compounds useful for therapy in mammals, and in particular for modulating CSK for treatment of various diseases associated with the overexpression of CSK.

### BACKGROUND

In an immune response, T cells are activated and initiate an intracellular signaling cascade that results in the release of cytotoxins and pro-inflammatory cytokines that ultimately result in the elimination of antigen-presenting disease cells. C-terminal Src kinase (CSK) negatively regulates proximal T-cell antigen receptor (TCR) signaling and T-cell function by phosphorylating LCK, which is a member of the Src family of tyrosine kinases. LCK (lymphocyte-specific kinase) is responsible for the activation of immune responses in T cells. More specifically, inhibition of CSK leads to dephosphorylation of LCK-pY505; increased phosphorylation of LCK-pY394; activation of downstream signaling pathways. LCK is regulated by phosphorylation on two conserved tyrosines wherein trans-autophosphorylation of its kinase domain activation loop tyrosine, Y394, increases its catalytic activity, and phosphorylation of its C-terminal inhibitory tyrosine, Y505, promotes its closed, inactive conformation. Moderate CSK inhibition can synergize with even weak TCR stimulation to enhance signaling/activation. Manz, et al. Small molecule inhibition of CSK alters affinity recognition by T cells. Manz et el., Small molecule inhibition of CSK alters affinity recognition of T cells, eLife 2015; 4:e08088, (2015). As an immuno-oncology agent, many tumor types may benefit.

Aberrant activation of the Src family of tyrosine kinases has been implicated in the development and progression of colorectal cancer (CRC). As a result, Src inhibitors are now being studied as possible therapeutic agents to treat metastatic disease.

Aberrant Src activation has been described in multiple cancers, including colorectal, ovarian, breast, lung, liver, prostate, and pancreatic cancers. (Gallick et al., Src family kinases in tumor progression and metastasis. (Lieu et al, The Src Family of Protein Tyrosine Kinases: A New and Promising Target for Colorectal Cancer Therapy, Clin Colorectal Cancer: 2010 April; 9(2): 89-94). In particular, gastrointestinal cancers show an increase in Src activity as the disease progresses, and chemoresistance in these cells appears to be related to an increase in motility, invasiveness, and detachment as a result of an increased activation of Src. Id.

It has been observed in preclinical studies that there was overexpression of CSK in more than 70% of renal cell carcinoma (RCC) specimens. (Feng et al., Overexpression of CSK-binding protein contributes to renal cell carcinogenesis, Oncogene (2009) 28, 3320-3331)

Compounds that can inhibit CSK, thereby activating the T-cell response, represent a new class of potential therapeutics capable of modulating the immune response and tumor growth. As there are no CSK inhibitors that are currently approved for the treatment or prevention of diseases in humans, there is an unmet need for new compounds that are capable of modulating CSK.

### SUMMARY

One embodiment of the disclosure relates to novel compounds, as described in any of the embodiments herein, or a pharmaceutically acceptable salt, a tautomer, a stereoisomer or a deuterated analog thereof, wherein these novel compounds can modulate CSK.

Another embodiment of this disclosure relates to a compound of Formula (I):

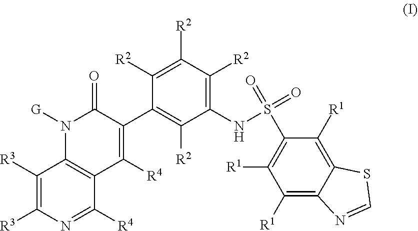

or a pharmaceutically acceptable salt, a tautomer, a stereoisomer, or a deuterated analog thereof, wherein R¹, R², R³, R⁴, and G are as described in any of the embodiments (including any of the subembodiments thereof) in this disclosure.

Other embodiments and sub-embodiments of Formula (I) are further described herein in this disclosure.

Another embodiment of the disclosure relates to a pharmaceutical composition comprising a compound according to Formula (I) or any embodiment and sub-embodiment of Formula (I) described herein in this disclosure, or a pharmaceutically acceptable salt, a tautomer, a stereoisomer or a deuterated analog of any of these compounds, and a pharmaceutically acceptable carrier or excipient.

Another embodiment of the disclosure relates to a pharmaceutical composition comprising a compound according to Formula (I), or any embodiment of Formula (I) described herein in this disclosure, or a pharmaceutically acceptable salt, a tautomer, a stereoisomer or a deuterated analog of any of these compounds, and another therapeutic agent.

Another embodiment of this disclosure relates to a method for treating a subject with a disease or condition mediated, at least in part, by CSK or T-cell activation, said method comprising administering to the subject an effective amount of a compound according to Formula (I), or any embodiment of Formula (I) described in this disclosure, or a pharmaceutically acceptable salt, a tautomer, a stereoisomer or a deuterated analog of any of these compounds, or a pharmaceutical composition of any of the compounds as described in this disclosure. In some embodiments of the method for treating a subject with a disease or condition mediated by mediated by CSK or T-cell activation are methods wherein there is selective inhibition of CSK over LCK.

Also provided herein is the use of a compound according to Formula (I), or any embodiment of Formula (I) described in this disclosure, or a pharmaceutically acceptable salt, a tautomer, a stereoisomer or a deuterated analog of any of these compounds, or a pharmaceutical composition of any of the compounds as described in this disclosure, for the treatment of a disease or condition mediated by mediated by CSK or T-cell activation. In some of such embodiments, the disease or condition mediated by CSK or T-cell activation is a disease or condition wherein there is selective inhibition of CSK over LCK.

Additional embodiments are described are further described in the Detailed Description of this disclosure.

### DETAILED DESCRIPTION

### I. Definitions

As used herein the following definitions apply unless clearly indicated otherwise:

It is noted here that as used herein and the appended claims, the singular forms “a,” “an,” and “the” include plural reference unless the context clearly dictates otherwise.

Unless a point of attachment indicates otherwise, the chemical moieties listed in the definitions of the variables of Formula (I) of this disclosure, and all the embodiments thereof, are to be read from left to right, wherein the right hand side is directly attached to the parent structure as defined. However, if a point of attachment (e.g., a dash “-”) is shown on the left hand side of the chemical moiety (e.g., -alkyloxy-C₁-C₆alkyl), then the left hand side of this chemical moiety is attached directly to the parent moiety as defined.

It is assumed that when considering generic descriptions of compounds described herein for the purpose of constructing a compound, such construction results in the creation of a stable structure. That is, one of ordinary skill in the art would recognize that, theoretically, some constructs would not normally be considered as stable compounds (that is, sterically practical and/or synthetically feasible).

“Alkyl,” by itself, or as part of another substituent, means, unless otherwise stated, a straight or branched chain hydrocarbon, having the number of carbon atoms designated (i.e. C₁-C₆ means one to six carbons). Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like. For each of the definitions herein (e.g., alkyl, alkoxy, arylalkyl, cycloalkylalkyl, heterocycloalkylalkyl, heteroarylalkyl, etc.), when a prefix is not included to indicate the number of carbon atoms in an alkyl portion, the alkyl moiety or portion thereof will have 12 or fewer main chain carbon atoms or 8 or fewer main chain carbon atoms or 6 or fewer main chain carbon atoms. For example, C₁-C₆alkyl refers to a straight or branched hydrocarbon having 1, 2, 3, 4, 5 or 6 carbon atoms and includes, but is not limited to, —CH₃, C₂alkyl, C₃alkyl, C₄alkyl, C₅alkyl, C₆alkyl, C₁-C₂alkyl, C₂alkyl, C₃alkyl, C₁-C₃alkyl, C₁-C₄alkyl, C₁-C₅alkyl, C₁-C₆alkyl, C₂-C₃alkyl, C₂-C₄alkyl, C₂-C₅alkyl, C₂-C₆alkyl, C₃-C₄alkyl, C₃-C₅alkyl, C₃-C₆alkyl, C₄-C₅alkyl, C₄-C₆alkyl, C₅-C₆ alkyl and C₆alkyl. While it is understood that substitutions are attached at any available atom to produce a stable compound, when optionally substituted alkyl is an R group of a moiety such as —OR (e.g. alkoxy), —SR (e.g. thioalkyl), —NHR (e.g. alkylamino), —C(O)NHR, and the like, substitution of the alkyl R group is such that substitution of the alkyl carbon bound to any O, S, or N of the moiety (except where N is a heteroaryl ring atom) excludes substituents that would result in any O, S, or N of the substituent (except where N is a heteroaryl ring atom) being bound to the alkyl carbon bound to any O, S, or N of the moiety.

“Alkylene” by itself or as part of another substituent means a linear or branched saturated divalent hydrocarbon moiety derived from an alkane having the number of carbon atoms indicated in the prefix. For example, (i.e., C₁-C₆ means one to six carbons; C₁-C₆alkylene is meant to include methylene, ethylene, propylene, 2-methylpropylene, pentylene, hexylene and the like). C₁₋₄ alkylene includes methylene —CH₂—, ethylene —CH₂CH₂—, propylene —CH₂CH₂CH₂—, and isopropylene —CH(CH₃)CH₂—, —CH₂CH(CH₃)—, —CH₂—(CH₂)₂CH₂—, —CH₂—CH(CH₃)CH₂—, —CH₂—C(CH₃)₂—CH₂—CH₂CH(CH₃)—. Typically, an alkyl (or alkylene) group will have from 1 to 24 carbon atoms, with those groups having 10 or fewer, 8 or fewer, or 6 or fewer carbon atoms. When a prefix is not included to indicate the number of carbon atoms in an alkylene portion, the alkylene moiety or portion thereof will have 12 or fewer main chain carbon atoms or 8 or fewer main chain carbon atoms, 6 or fewer main chain carbon atoms, or 4 or fewer main chain carbon atoms, or 3 or fewer main chain carbon atoms, or 2 or fewer main chain carbon atoms, or 1 carbon atom.

“Alkoxy” or “alkoxyl” refers to a —O-alkyl group, where alkyl is as defined herein. By way of example, “C₁-C₆alkoxy” refers to a —O—C₁-C₆alkyl group, where alkyl is as defined herein. While it is understood that substitutions on alkoxy are attached at any available atom to produce a stable compound, substitution of alkoxy is such that O, S, or N (except where N is a heteroaryl ring atom), are not bound to the alkyl carbon bound to the alkoxy O. Further, where alkoxy is described as a substituent of another moiety, the alkoxy oxygen is not bound to a carbon atom that is bound to an O, S, or N of the other moiety (except where N is a heteroaryl ring atom), or to an alkene or alkyne carbon of the other moiety.

The terms “alkoxyalkyl” and “alkoxyalkylene” refer to an alkyl group substituted with an alkoxy group. By way of example, “C₁-C₆alkoxyC₁-C₆alkyl” refers to C₁-C₆alkyl substituted with a C₁-C₆alkoxy where alkyl and alkoxy are as defined herein, while “C₁-C₃alkoxyC₁-C₃alkylene” refers to C₁-C₃alkyl substituted with a C₁-C₃alkoxy where alkylene and alkoxy are as defined herein.

“Amino” or “amine” denotes the group NH₂.

“Aryl” by itself, or as part of another substituent, unless otherwise stated, refers to a monocyclic, bicyclic or polycyclic polyunsaturated aromatic hydrocarbon radical containing 6 to 14 ring carbon atoms, which can be a single ring or multiple rings (up to three rings) which are fused together or linked covalently. Aryl, however, does not encompass or overlap in any way with heteroaryl defined below. If one or more aryl rings are fused with a heteroaryl ring, the resulting ring system is heteroaryl. Non-limiting examples of unsubstituted aryl groups include phenyl, 1-naphthyl and 2-naphthyl. The term “arylene” refers to a divalent aryl, wherein the aryl is as defined herein.

“Arylalkyl” and “arylalkylene” refer to an -(alkylene)-aryl group where alkylene as defined herein has the indicated number of carbon atoms or if unspecified having six or fewer carbon atoms; and aryl is as defined herein. By way of example, aryl-C₁-C₆alkyl refers to an aryl ring attached to an alkylene chain with 1-6 carbon atoms, wherein the alkylene chain is attached to the parent moiety. Non limiting examples of arylalkyl groups include benzyl, napthylphenyl, and the like.

“5-6 membered aromatic ring” refers to a phenyl ring or a 5-6 membered heteroaryl ring as defined herein.

“Cycloalkyl” or “Carbocycle” or “Carbocyclic” by itself, or as part of another substituent, unless otherwise stated, refers to saturated or partially unsaturated, nonaromatic monocyclic ring, or fused rings, such as bicyclic or tricyclic carbon ring systems, or cubane, having the number of carbon atoms indicated in the prefix or if unspecified having 3-6, also 4-6, and also 5-6 ring members per ring, such as cyclopropyl, cyclopentyl, cyclohexyl, where one or two ring carbon atoms may optionally be replaced by a carbonyl. Further, the term cycloalkyl is intended to encompass ring systems fused to an aromatic ring (e.g., of an aryl or heteroaryl), regardless of the point of attachment to the remainder of the molecule. Cycloalkyl refers to hydrocarbon rings having the indicated number of ring atoms (e.g., C₃_₆ cycloalkyl and 3-6 membered cycloalkyl both mean three to six ring carbon atoms). The term “cycloalkenyl” refers to a cycloalkyl having at least one unit of unsaturation. A substituent of a cycloalkyl or cycloalkenyl may be at the point of attachment of the cycloalkyl or cycloalkenyl group, forming a quaternary center.

“Cycloalkylalkyl” and “cycloalkylalkylene” refer to an -(alkylene)-cycloalkyl group where alkylene as defined herein has the indicated number of carbon atoms or if unspecified having six or fewer carbon atoms; and cycloalkyl is as defined herein has the indicated number of carbon atoms or if unspecified having 3-10, also 3-8, and also 3-6, ring members per ring. By way of example, 4-6 membered cycloalkyl-C₁-C₆alkyl refers to a cycloalkyl with 4-6 carbon atoms attached to an alkylene chain with 1-6 carbon atoms, wherein the alkylene chain is attached to the parent moiety. Other exemplary cycloalkylalkyl includes, e.g., cyclopropylmethylene, cyclobutylethylene, cyclobutylmethylene, and the like.

The term “cyano” refers to the group —CN. The term “C₁-C₆cyanoalkyl” refers to a C₁-C₆alkyl, as defined herein, that is substituted with 1, 2 or 3 cyano groups. “C₁-C₆cyanoalkylethynylene” is a group —C≡C—C₁-C₆cyanoalkyl.

“Halogen” or “halo” refers to all halogens, that is, chloro (Cl), fluoro (F), bromo (Br), or iodo (I).

“Heteroatom” is meant to include oxygen (O), nitrogen (N), and sulfur (S).

“Heteroaryl” refers to a monocyclic or bicyclic aromatic ring radical containing 5-9 ring atoms (also referred to in this disclosure as a 5-9 membered heteroaryl, including monocyclic aromatic ring radicals containing 5 or 6 ring atoms (also referred to in this disclosure as a 5-6 membered heteroaryl), containing one or more, 14, 13, or 12, heteroatoms independently selected from the group consisting of O, S, and N. Any aromatic ring or ring system containing at least one heteroatom is a heteroaryl regardless of the point of attachment (i.e., through any one of the fused rings). Heteroaryl is also intended to include oxidized S or N, such as sulfinyl, sulfonyl and Noxide of a tertiary ring nitrogen. A carbon or nitrogen atom is the point of attachment of the heteroaryl ring structure such that a stable compound is produced. Examples of heteroaryl groups include, but are not limited to, pyridyl, pyridazinyl, pyrazinyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, indolyl, triazinyl, quinoxalinyl, cinnolinyl, phthalazinyl, benzotriazinyl, benzimidazolyl, benzopyrazolyl, benzotriazolyl, benzisoxazolyl, isobenzofuryl, isoindolyl, indolizinyl, benzotriazinyl, thienopyridyl, thienopyrimidinyl, pyrazolopyrimidinyl, imidazopyridines, benzothiaxolyl, benzothienyl, quinolyl, isoquinolyl, indazolyl, pteridinyl and thiadiazolyl. “Nitrogen containing heteroaryl” refers to heteroaryl wherein at least one of the ring heteroatoms is N.

“Heteroarylalkyl” and “heteroarylalkylene” refer to an -(alkylene)-heteroaryl group where alkylene as defined herein has the indicated number of carbon atoms or if unspecified having six or fewer carbon atoms; and heteroaryl is as defined herein. By way of example, 5-9 membered heteroaryl-C₁-C₆alkyl refers to a heteroaryl ring with 5-9 ring members attached to an alkylene chain with 1-6 carbon atoms, wherein the alkylene chain is attached to the parent moiety. Non limiting examples of heteroarylalkyl groups include pyridylmethyl, pyrazolylethyl, thiazolylmethyl, and the like.

“Heterocycloalkyl” refers to a saturated or partially unsaturated non-aromatic cycloalkyl group that contains from one to five heteroatoms selected from N, O, S (including S(O) and S(O)₂), or P (including phosphine oxide) wherein the nitrogen, sulfur, and phosphorous atoms are optionally oxidized, and the nitrogen atom(s) are optionally quarternized, the remaining ring atoms being C, where one or two C atoms may optionally be present as a carbonyl. Further, the term heterocycloalkyl is intended to encompass any ring or ring system containing at least one heteroatom that is not a heteroaryl, regardless of the point of attachment to the remainder of the molecule. Heterocycloalkyl groups include those having a ring with a formally charge-separated aromatic resonance structure, for example, N-methylpyridonyl. The heterocycloalkyl may be substituted with one or two oxo groups, and can include sulfone and sulfoxide derivatives. The heterocycloalkyl may be a monocyclic, a fused bicyclic or a fused polycyclic ring system of 3 to 12, 4 to 10, 5 to 10, or 5 to 6 ring atoms in which one to five ring atoms are heteroatoms selected from —N═, —N—, —O—, —S—, —S(O)—, or —S(O)₂— and further wherein one or two ring atoms are optionally replaced by a —C(O)— group. As an example, a 4-6 membered heterocycloalkyl is a heterocycloalkyl with 4-6 ring members having at least one heteroatom. The heterocycloalkyl can also be a heterocyclic alkyl ring fused with a cycloalkyl. Non limiting examples of heterocycloalkyl groups include pyrrolidinyl, piperidinyl, morpholinyl, pyridonyl, and the like. A heterocycloalkyl group can be attached to the remainder of the molecule through a ring carbon or a heteroatom. “Heterocycloalkenyl” refers to a heterocycloalkyl having at least one unit of unsaturation. A substituent of a heterocycloalkyl or heterocycloalkenyl may be at the point of attachment of the heterocycloalkyl or heterocycloalkenyl group, forming a quaternary center.

“Heterocycloalkylalkyl” and “heterocycloalkylalkylene” refer to an -(alkylene)-heterocycloalkyl group where alkylene as defined herein has the indicated number of carbon atoms or if unspecified having six or fewer carbon atoms; and heterocycloalkyl is as defined herein. By way of example, 4-6 membered heterocycloalkyl-C₁-C₆alkyl refers to a heterocycloalkyl with 4-6 ring members attached to an alkylene chain with 1-6 carbon atoms, wherein the alkylene chain is attached to the parent moiety. Non limiting examples of heterocycloalkylalkyl groups include pyrrolidinylmethyl, piperidinylmethyl, morpholinylethyl, pyridonylmethyl, and the like.

“Hydroxyl” or “hydroxy” refers to the group OH. The term “hydroxyalkyl” or “hydroxyalkylene” refers to an alkyl group or alkylene group, respectively as defined herein, substituted with 1-5 hydroxy groups.

“Optional substituents” or “optionally substituted” as used throughout the disclosure means that the substitution on a compound may or may not occur, and that the description includes instances where the substitution occurs and instances in which the substitution does not. For example, the phrase “optionally substituted with 1-3 T^{l} groups” means that the T^{l} group may but need not be present. It is assumed in this disclosure that optional substitution on a compound occurs in a way that would result in a stable compound.

Selective inhibition of CSK over LCK means CSK is inhibited to a greater extent, which can be measured by assays for CSK IC₅₀ values as described herein, compared to inhibition of LCK, which can be measured by assays for LCK IC₅₀ values as described herein.

As used herein in connection with compounds of the disclosure, the term “synthesizing” and like terms means chemical synthesis from one or more precursor materials.

As used herein, the term “composition” refers to a formulation suitable for administration to an intended animal subject for therapeutic purposes that contains at least one pharmaceutically active compound and at least one pharmaceutically acceptable carrier or excipient.

The term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile, e.g., for injectables.

“Pharmaceutically acceptable salt” refers to a salt which is acceptable for administration to a patient, such as a mammal (e.g., salts having acceptable mammalian safety for a given dosage regime). Contemplated pharmaceutically acceptable salt forms include, without limitation, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug. Such salts can be derived from pharmaceutically acceptable inorganic or organic bases and from pharmaceutically-acceptable inorganic or organic acids, depending on the particular substituents found on the compounds described herein.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. In another example, a salt can be prepared by reacting the free base and acid in an organic solvent.

When compounds of the present disclosure contain relatively acidic functionalities, base addition salts can be obtained by contacting the neutral form of such compounds with a sufficient amount of the desired base (i.e. a primary, secondary, tertiary, quaternary, or cyclic amine; an alkali metal hydroxide; alkaline earth metal hydroxide; or the like), either neat or in a suitable inert solvent. The desired acid can be, for example, a pyranosidyl acid (such as glucuronic acid or galacturonic acid), an alpha-hydroxy acid (such as citric acid or tartaric acid), an amino acid (such as aspartic acid or glutamic acid), an aromatic acid (such as benzoic acid or cinnamic acid), a sulfonic acid (such as p-toluenesulfonic acid or ethanesulfonic acid), or the like. In some embodiments, salts can be derived from pharmaceutically acceptable acids such as acetic, trifluoroacetic, propionic, ascorbic, benzenesulfonic, benzoic, camphorsulfonic, citric, ethanesulfonic, fumaric, glycolic, gluconic, glucoronic, glutamic, hippuric, hydrobromic, hydrochloric, isethionic, lactic, lactobionic, maleic, malic, malonic, mandelic, oxalic, methanesulfonic, mucic, naphthalenesulfonic, nicotinic, nitric, pamoic, pantothenic, phosphoric, succinic, sulfuric, sulfamic, hydroiodic, carbonic, tartaric, p-toluenesulfonic, pyruvic, aspartic, benzoic, cinnamic, anthranilic, mesylic, salicylic, p-hydroxybenzoic, phenylacetic, embonic (pamoic), ethanesulfonic, benzenesulfonic, 2-hydroxyethanesulfonic, sulfanilic, stearic, cyclohexylsulfamic, cyclohexylaminosulfonic, quinic, algenic, hydroxybutyric, galactaric and galacturonic acid and the like.

Also included are salts of amino acids such as arginate and the like, and salts of organic acids like glucuronic or galactunoric acids and the like (see, for example, Berge, S. M. et al., “Pharmaceutical Salts,” J. Pharmaceutical Science, 1977, 66:1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The pharmaceutically acceptable salt of the different compounds may be present as a complex. Examples of complexes include 8-chlorotheophylline complex (analogous to, e.g., dimenhydrinate: diphenhydramine 8-chlorotheophylline (1:1) complex; Dramamine) and various cyclodextrin inclusion complexes.

The term “deuterated” as used herein alone or as part of a group, means substituted deuterium atoms. The term “deuterated analog” as used herein alone or as part of a group, means substituted deuterium atoms in place of hydrogen. The deuterated analog of the disclosure may be a fully or partially deuterium substituted derivative. In some embodiments, the deuterium substituted derivative of the disclosure holds a fully or partially deuterium substituted alkyl, aryl or heteroaryl group.

The disclosure also embraces isotopically-labeled compounds of the present disclosure which are identical to those recited herein, but for the fact that one or more atoms are replaced by an atom having an atomic mass or mass number different from the atomic mass or mass number usually found in nature. All isotopic variations of the compounds of the present disclosure, whether radioactive or not, are intended to be encompassed within the scope of the present disclosure. Examples of isotopes that can be incorporated into compounds of the disclosure include isotopes of hydrogen, carbon, nitrogen, oxygen, phosphorous, fluorine, and chlorine, such as, but not limited to ²H (deuterium, D), ³H (tritium), ¹¹C, ¹³C, ⁴C, ⁵N, ¹⁸F, ³¹P, ³²P ³⁵S, ³⁶Cl, and ¹²⁵I. Unless otherwise stated, when a position is designated specifically as “H” or “hydrogen,” the position is understood to have hydrogen at its natural abundance isotopic composition or its isotopes, such as deuterium (D) or tritium (3H). Certain isotopically-labeled compounds of the present disclosure (e.g., those labeled with ³H and ¹⁴C) are useful in compound and/or substrate tissue distribution assays. Tritiated (i.e., ³H) and carbon-14 (i.e., ¹⁴C) and fluorine-18 (¹⁸F) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those described in the Schemes and in the Examples herein below, by substituting an isotopically labeled reagent for a non-isotopically labeled reagent.

“Prodrugs” means any compound which releases an active parent drug according to Formula (I) in vivo when such prodrug is administered to a subject. Prodrugs of a compound of Formula (I) are prepared by modifying functional groups present in the compound of Formula (I) in such a way, either in routine manipulation or in vivo, that the modifications may be cleaved in vivo to release the parent compound. Prodrugs may proceed from prodrug form to active form in a single step or may have one or more intermediate forms which may themselves have activity or may be inactive. Some prodrugs are activated enzymatically to yield the active compound, or a compound which, upon further chemical reaction, yields the active compound. Prodrugs include compounds of Formula (I) wherein a hydroxy, amino, carboxyl or sulfhydryl group in a compound of Formula (I) is bonded to any group that may be cleaved in vivo to regenerate the free hydroxyl, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds of Formula (I), and the like. Other examples of prodrugs include, without limitation, carbonates, ureides, solvates, or hydrates of the active compound. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

As described in The Practice of Medicinal Chemistry, Ch. 31-32 (Ed. Wermuth, Academic Press, San Diego, Calif., 2001), prodrugs can be conceptually divided into two non-exclusive categories, bioprecursor prodrugs and carrier prodrugs. Generally, bioprecursor prodrugs are compounds that are inactive or have low activity compared to the corresponding active drug compound, that contain one or more protective groups and are converted to an active form by metabolism or solvolysis. Both the active drug form and any released metabolic products should have acceptably low toxicity. Typically, the formation of active drug compound involves a metabolic process or reaction that is one of the follow types:

(1) Oxidative reactions: Oxidative reactions are exemplified without limitation to reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

(2) Reductive reactions: Reductive reactions are exemplified without limitation to reactions such as reduction of carbonyl functionalities, reduction of alcohol functionalities and carbon-carbon double bonds, reduction of nitrogen-containing functional groups, and other reduction reactions.

(3) Reactions without change in the oxidation state: Reactions without change in the state of oxidation are exemplified without limitation to reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. (See, e.g., Cheng et al., U.S. Patent Publ. No. 2004/0077595, incorporated herein by reference.) Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g. stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols.

The term “carrier” is also meant to include microspheres, liposomes, micelles, nanoparticles (naturally-equipped nanocarriers, for example, exosomes), and the like. It is known that exosomes can be highly effective drug carriers, and there are various ways in which drugs can be loaded into exosomes, including those techniques described in J Control Release. 2015 Dec. 10; 219: 396-405, the contents of which are incorporated by reference in its entirety.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic process in the body of a subject. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques known in the art. See, e.g., Bertolini et al., 1997, J. Med. Chem., 40:2011-2016; Shan et al., 1997, J Pharm Sci 86(7):756-757; Bagshawe, 1995, Drug Dev. Res., 34:220-230.

“Tautomer” means compounds produced by the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The tautomers also refer to one of two or more structural isomers that exist in equilibrium and are readily converted from one isomeric form to another. Examples of include keto-enol tautomers, such as acetone/propen-2-ol, imine-enamine tautomers and the like, ring-chain tautomers, such as glucose/2,3,4,5,6-pentahydroxy-hexanal and the like, the tautomeric forms of heteroaryl groups containing a —N═C(H)—NH— ring atom arrangement, such as pyrazoles, imidazoles, benzimidazoles, triazoles, and tetrazoles. Where the compound contains, for example, a keto or oxime group or an aromatic moiety, tautomeric isomerism (‘tautomerism’) can occur. The compounds described herein may have one or more tautomers and therefore include various isomers. A person of ordinary skill in the art would recognize that other tautomeric ring atom arrangements are possible. All such isomeric forms of these compounds are expressly included in the present disclosure.

“Isomers” mean compounds that have identical molecular Formulae but differ in the nature or sequence of bonding of their atoms or in the arrangement of their atoms in space. Isomers that differ in the arrangement of their atoms in space are termed “stereoisomers.” “Stereoisomer” and “stereoisomers” refer to compounds that exist in different stereoisomeric forms, for example, if they possess one or more asymmetric centers or a double bond with asymmetric substitution and, therefore, can be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers.” When a compound has an asymmetric center, for example, an atom such as carbon bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.” As another example, stereoisomers include geometric isomers, such as cis- or trans-orientation of substituents on adjacent carbons of a double bond. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 6th edition J. March, John Wiley and Sons, New York, 2007) differ in the chirality of one or more stereocenters.

In the context of the use, testing, or screening of compounds that are or may be modulators, the term “contacting” means that the compound(s) are caused to be in sufficient proximity to a particular molecule, complex, cell, tissue, organism, or other specified material that potential binding interactions and/or chemical reaction between the compound and other specified material can occur.

By “assaying” is meant the creation of experimental conditions and the gathering of data regarding a particular result of the exposure to specific experimental conditions. For example, enzymes can be assayed based on their ability to act upon a detectable substrate. A compound can be assayed based on its ability to bind to a particular target molecule or molecules.

As used herein, the terms “ligand” and “modulator” are used equivalently to refer to a compound that changes (i.e., increases or decreases) the activity of a target biomolecule, e.g., an enzyme such as those described herein. Generally a ligand or modulator will be a small molecule, where “small molecule refers to a compound with a molecular weight of 1500 Daltons or less, 1000 Daltons or less, 800 Daltons or less, or 600 Daltons or less. Thus, an “improved ligand” is one that possesses better pharmacological and/or pharmacokinetic properties than a reference compound, where “better” can be defined by one skilled in the relevant art for a particular biological system or therapeutic use.

The term “binds” in connection with the interaction between a target and a potential binding compound indicates that the potential binding compound associates with the target to a statistically significant degree as compared to association with proteins generally (i.e., non-specific binding). Thus, the term “binding compound” refers to a compound that has a statistically significant association with a target molecule. In some embodiments, a binding compound interacts with a specified target with a dissociation constant (K_{D}) of 10 mM or less, 1,000 μM or less, 100 μM or less, 10 μM or less, 1 μM or less, 1,000 nM or less, 100 nM or less, 10 nM or less, or 1 nM or less. In the context of compounds binding to a target, the terms “greater affinity” and “selective” indicates that the compound binds more tightly than a reference compound, or than the same compound in a reference condition, i.e., with a lower dissociation constant. In some embodiments, the greater affinity is at least 2, 3, 4, 5, 8, 10, 50, 100, 200, 400, 500, 1000, or 10,000-fold greater affinity.

The terms “modulate,” “modulation,” and the like refer to the ability of a compound to increase or decrease the function and/or expression of a target, such as CSK, where such function may include transcription regulatory activity and/or binding. Modulation may occur in vitro or in vivo. Modulation, as described herein, includes the inhibition, antagonism, partial antagonism, activation, agonism or partial agonism of a function or characteristic associated with CSK, either directly or indirectly, and/or the upregulation or downregulation of the expression CSK, either directly or indirectly. In another embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction.

As used herein, the terms “treat,” “treating,” “therapy,” “therapies,” and like terms refer to the administration of material, e.g., any one or more compound(s) as described herein in an amount effective to inhibit CSK or to activate T-cells. In other embodiments, the terms “treat,” “treating,” “therapy,” “therapies,” and like terms refer to the administration of material, e.g., any one or more compound(s) as described herein is an amount effective to prevent, alleviate, or ameliorate one or more symptoms of a disease or condition, i.e., indication, and/or to prolong the survival of the subject being treated.

The terms “prevent,” “preventing,” “prevention” and grammatical variations thereof as used herein, refers to a method of partially or completely delaying or precluding the onset or recurrence of a disease, disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “subject,” “animal subject,” and the like refers to a living organism including, but not limited to, human and non-human vertebrates, e.g. any mammal, such as a human, other primates, sports animals and animals of commercial interest such as cattle, horses, ovines, or porcines, rodents, or pets such as dogs and cats.

“Unit dosage form” refers to a composition intended for a single administration to treat a subject suffering from a disease or medical condition. Each unit dosage form typically comprises each of the active ingredients of this disclosure plus pharmaceutically acceptable excipients. Examples of unit dosage forms are individual tablets, individual capsules, bulk powders, liquid solutions, ointments, creams, eye drops, suppositories, emulsions or suspensions. Treatment of the disease or condition may require periodic administration of unit dosage forms, for example: one unit dosage form two or more times a day, one with each meal, one every four hours or other interval, or only one per day. The expression “oral unit dosage form” indicates a unit dosage form designed to be taken orally.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

In the present context, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. In general, satisfactory results in subjects are indicated to be obtained at a daily dosage of from about 0.1 to about 10 g/kg subject body weight. In some embodiments, a daily dose ranges from about 0.10 to 10.0 mg/kg of body weight, from about 1.0 to 3.0 mg/kg of body weight, from about 3 to 10 mg/kg of body weight, from about 3 to 150 mg/kg of body weight, from about 3 to 100 mg/kg of body weight, from about 10 to 100 mg/kg of body weight, from about 10 to 150 mg/kg of body weight, or from about 150 to 1000 mg/kg of body weight. The dosage can be conveniently administered, e.g., in divided doses up to four times a day or in sustained-release form.

The ability of a compound to inhibit the function of CSK can be demonstrated in a biochemical assay, e.g., binding assay, or a cellbased assay.

The ability of a compound to inhibit the function of LCK can be demonstrated in a biochemical assay, e.g., binding assay, or a cellbased assay.

As used herein, the term “CSK mediated disease or condition” refers to a disease or condition in which the biological function of CSK affect the development and/or course of the disease or condition, and/or in which modulation of CSK alters the development, course, and/or symptoms. A CSK mediated disease or condition includes a disease or condition for which CSK inhibition provides a therapeutic benefit, e.g. wherein treatment with CSK inhibitors, including compounds described herein, provides a therapeutic benefit to the subject suffering from or at risk of the disease or condition. A CSK mediated disease or condition is intended to include a cancer that harbors loss of function mutations in CSK, or a cancer where there is activation of CSK. A CSK mediated disease or condition is also intended to include various human carcinomas, including those of colon, lung, pancreas, and ovary, as well as diseases or conditions associated with tumor neovascularization, and invasiveness.

Also in the context of compounds binding to a biomolecular target, the term “greater specificity” indicates that a compound binds to a specified target to a greater extent than to another biomolecule or biomolecules that may be present under relevant binding conditions, where binding to such other biomolecules produces a different biological activity than binding to the specified target. Typically, the specificity is with reference to a limited set of other biomolecules, e.g., in the case of CSK. In particular embodiments, the greater specificity is at least 2, 3, 4, 5, 8, 10, 50, 100, 200, 400, 500, or 1000-fold greater specificity.

As used herein in connection with binding compounds or ligands, the term “specific for CSK,” and terms of like import mean that a particular compound binds to CSK to a statistically greater extent than to other epigenetic targets that may be present in a particular sample. Also, where biological activity other than binding is indicated, the term “specific for CSK” indicates that a particular compound has greater biological effect associated with binding CSK than to other enzymes, e.g., enzyme activity inhibition.

The term “first line cancer therapy” refers to therapy administered to a subject as an initial regimen to reduce the number of cancer cells. First line therapy is also referred to as induction therapy, primary therapy and primary treatment. First-line therapy can be an administered combination with one or more agents. A summary of currently accepted approaches to first line treatment for certain disease can be found in the NCI guidelines for such diseases.

The term “second line cancer therapy” refers to a cancer treatment that is administered to a subject who does not respond to first line therapy, that is, often first line therapy is administered or who has a recurrence of cancer after being in remission. In certain embodiments, second line therapy that may be administered includes a repeat of the initial successful cancer therapy, which may be any of the treatments described under “first line cancer therapy.” A summary of the currently accepted approaches to second line treatment for certain diseases is described in the NCI guidelines for such diseases.

The term “refractory” refers to wherein a subject fails to respond or is otherwise resistant to cancer therapy or treatment. The cancer therapy may be first-line, second-line or any subsequently administered treatment. In certain embodiments, refractory refers to a condition where a subject fails to achieve complete remission after two induction attempts. A subject may be refractory due to a cancer cell's intrinsic resistance to a particular therapy, or the subject may be refractory due to an acquired resistance that develops during the course of a particular therapy.

In addition, abbreviations as used herein have respective meanings as follows:

| | |
|---|---|
| ° C. | Degree Celsius |
| AcOH | Acetic acid |
| BOC | tert-butoxycarbonyl |
| DMF | Dimethylformamide |
| DMSO | Dimethylsulfoxide |
| EtOAc | Ethyl Acetate |
| ESI | Electrospray ionization |
| HPLC | High Performance Liquid Chromatography |
| IC₅₀ | Half minimal (50%) inhibitory concentration |
| LCMS | Liquid Chromatography Mass Spectrometry |
| [M + H+]+ or (MH)+ | Mass peak plus hydrogen |
| [M − H−]− or (MH)− | Mass peak minus hydrogen |
| Me | Methyl |
| MeOH | Methanol |
| MS | Mass spectrometry |
| N | Normal |
| PMB | (4-methoxyphenyl)methanamine or para- |
| | methoxy benzyl |
| RP | Reverse phase |
| RT | Room temperature |
| TLC | Thin-layer chromatography |
| THF | Tetrahydrofuran |
| XantPhos | 4,5-bis(diphenylphosphino)-9,9- |
| | dimethylxanthene |

### II. Compounds

Embodiment 1 of this disclosure relates to a compound having Formula (I):

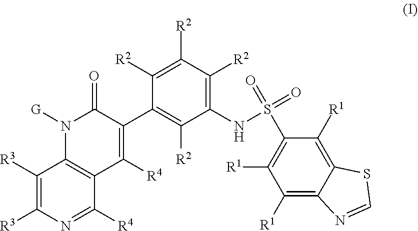

or a pharmaceutically acceptable salt, a tautomer, a stereoisomer, or a deuterated analog thereof, wherein:

G is H, alkyl substituted with 0-3 X³ groups and 0-1 Z¹ group, -[C(X¹)(X²)]₁₋₆-alkoxy substituted with 0-4 X³ groups and 0-1 Z¹ group, -[C(X¹)(X²)]₀₋₆-cycloalkyl substituted with 0-4 X⁴ groups and 0-1 Z¹ group, or —[C(X¹)(X²)]₀₋₆-heterocycloalkyl substituted with 0-3 X⁴ groups and 0-1 Z¹ group;

each R¹ is independently H, halogen, OH, CN, C₁-C₃alkyl optionally substituted with 1-3 Z² groups, or alkoxy optionally substituted with 1-3 Z² groups;

each R² is independently H, halogen, CN, C₁-C₃alkyl optionally substituted with 1-3 Z² groups, or C₁-C₃alkoxy optionally substituted with 1-3 Z² groups;

each R³ is independently H, CN, halogen, —NHR⁵, alkyl optionally substituted with 1-4 Z² groups, alkoxy optionally substituted with 1-4 Z² groups, alkoxyalkyl optionally substituted with 1-4 Z² groups, cycloalkyl optionally substituted with 1-4 Z³ groups, cycloalkylalkyl optionally substituted with 1-4 Z³, heterocycloalkyl optionally substituted with 1-4 Z³ groups, heterocycloalkylalkyl optionally substituted with 1-4 Z³ groups, heteroaryl optionally substituted with 1-4 Z³ groups, heteroarylalkyl optionally substituted with 1-4 Z³ groups, aryl optionally substituted with 1-4 Z³ groups, or arylalkyl optionally substituted with 1-4 Z³ groups, provided that no more than one R³ is —NHR⁵;

each R⁴ is independently H, —C₁-C₃alkyl, halo, OH, or CN;

R⁵ is H, alkyl optionally substituted with 1-4 Z² groups, alkoxyalkyl optionally substituted with 1-4 Z² groups, cycloalkyl optionally substituted with 1-4 Z³ groups, cycloalkylalkyl optionally substituted with 1-4 Z³ groups, heterocycloalkyl optionally substituted with 1-4 Z³ groups, heterocycloalkylalkyl optionally substituted with 1-4 Z³ groups, heteroaryl optionally substituted with 1-4 Z³ groups, heteroarylalkyl optionally substituted with 1-4 Z³ groups, aryl optionally substituted with 1-4 Z³ groups, or arylalkyl optionally substituted with 1-4 Z³ groups provided that when R⁵ is heterocycloalkyl or heteroaryl, then any heteroatom of R⁵ cannot be attached to the nitrogen atom of —NHR⁵;

each X¹ is independently H, halogen or alkyl;

each X² is independently H, halogen or alkyl;

each X³ is independently OH, CN, or halogen;

each X⁴ is independently D, OH, CN, halogen, alkyl optionally substituted with 1-4 Z² groups, or alkoxy optionally substituted with 1-4 Z² groups;

Z¹ is cycloalkyl optionally substituted with 1-4 Z³ groups, cycloalkylalkyl optionally substituted with 1-4 Z³ groups, heterocycloalkyl optionally substituted with 1-4 Z³ groups, heterocycloalkylalkyl optionally substituted with 1-4 Z³ groups, heteroaryl optionally substituted with 1-4 Z³ groups, heteroarylalkyl optionally substituted with 1-4 Z³ groups, aryl optionally substituted with 1-4 Z³ groups, or arylalkyl optionally substituted with 1-4 Z³ groups;

each Z² is independently halogen or OH; and

each Z³ is independently halogen, OH, or alkyl optionally substituted with 1-3 halogens, provided that when Z³ is halogen or OH, then Z³ cannot be attached to any heteroatom from R⁵.

### Subembodiments of Embodiment 1

Embodiment 1(a) of this disclosure relates to Embodiment 1, wherein G is H.

Embodiment 1(b) of this disclosure relates to Embodiment 1, wherein G is alkyl substituted with 0-4 X³ groups and 0-1 Z¹ group.

Embodiment 1(c) of this disclosure relates to Embodiment 1, wherein G is —[C(X¹)(X²)]₁₋₆-alkoxy substituted with 0-4 X³ groups and 0-1 Z¹ group.

Embodiment 1(d) of this disclosure relates to Embodiment 1, wherein G is —[C(X¹)(X²)]₀₋₆-cycloalkyl substituted with 0-4 X⁴ groups and 0-1 Z¹ group.

Embodiment 1(e) of this disclosure relates to Embodiment 1, wherein G is —[C(X¹)(X²)]₀₋₆-heterocycloalkyl substituted with 0-3 X⁴ groups and 0-1 Z¹ group.

Embodiment 1(f) of this disclosure relates to any one of Embodiments 1, 1(a), 1(b), 1(c), 1(d), or 1(e), wherein each R¹ is independently H or halogen.

Embodiment 1(g) of this disclosure relates to any one of Embodiments 1, 1(a), 1(b), 1(c), 1(d), 1(e), or 1(f), wherein each R² is independently H or halogen.

Embodiment 1(h) of this disclosure relates to any one of Embodiments 1, 1(a), 1(b), 1(c), 1(d), 1(e), 1(f) or 1(g), wherein one R³ is H, and the other R³ is —NHR⁵

Embodiment 1(i) of this disclosure relates to any one of Embodiments 1, 1(a), 1(b), 1(c), 1(d), 1(e), 1(f) or 1(g), wherein one R³ is H, and the other R³ is —NHR⁵

Embodiment 1(j) of this disclosure relates to any one of Embodiments 1, 1(a), 1(b), 1(c), 1(d), 1(e), 1(f) or 1(g), wherein one R³ is CN, and the other R³ is —NHR.

Embodiment 1(k) of this disclosure relates to any one of Embodiments 1, 1(a), 1(b), 1(c), 1(d), 1(e), 1(f) or 1(g), wherein one R³ is halogen, and the other R³ is —NHR.

Embodiment 1(l) of this disclosure relates to any one of Embodiments 1, 1(a), 1(b), 1(c), 1(d), 1(e), 1(f) or 1(g), wherein one R³ is alkyl optionally substituted with 1-4 Z² groups, and the other R³ is —NHR⁵

Embodiment 1(m) of this disclosure relates to any one of Embodiments 1, 1(a), 1(b), 1(c), 1(d), 1(e), 1(f) or 1(g), wherein one R³ is alkoxy optionally substituted with 1-4 Z² groups, and the other R³ is —NHR⁵

Embodiment 1(n) of this disclosure relates to any one of Embodiments 1, 1(a), 1(b), 1(c), 1(d), 1(e), 1(f) or 1(g), wherein one R³ is alkoxy optionally substituted with 1-4 Z² groups, and the other R³ is —NHR⁵

Embodiment 1(o) of this disclosure relates to any one of Embodiments 1, 1(a), 1(b), 1(c), 1(d), 1(e), 1(f) or 1(g), wherein one R³ is alkoxyalkyl optionally substituted with 1-4 Z² groups, and the other R³ is —NHR⁵

Embodiment 1(p) of this disclosure relates to any one of Embodiments 1, 1(a), 1(b), 1(c), 1(d), 1(e), 1(f) or 1(g), wherein one R³ is alkoxyalkyl optionally substituted with 1-4 Z² groups, and the other R³ is —NHR⁵

Embodiment 1(q) of this disclosure relates to any one of Embodiments 1, 1(a), 1(b), 1(c), 1(d), 1(e), 1(f) or 1(g), wherein one R³ is cycloalkyl optionally substituted with 1-4 Z³ groups, and the other R³ is —NHR⁵

Embodiment 1(r) of this disclosure relates to any one of Embodiments 1, 1(a), 1(b), 1(c), 1(d), 1(e), 1(f) or 1(g), wherein one R³ is cycloalkylalkyl optionally substituted with 1-4 Z³ groups, and the other R³ is —NHR⁵

Embodiment 1(s) of this disclosure relates to any one of Embodiments 1, 1(a), 1(b), 1(c), 1(d), 1(e), 1(f) or 1(g), wherein one R³ is heterocycloalkyl optionally substituted with 1-4 Z³ groups, and the other R³ is —NHR⁵

Embodiment 1(t) of this disclosure relates to any one of Embodiments 1, 1(a), 1(b), 1(c), 1(d), 1(e), 1(f) or 1(g), wherein one R³ is heterocycloalkylalkyl optionally substituted with 1-4 Z³ groups, and the other R³ is —NHR⁵.

Embodiment 1(u) of this disclosure relates to any one of Embodiments 1, 1(a), 1(b), 1(c), 1(d), 1(e), 1(f) or 1(g), wherein one R³ is heteroaryl optionally substituted with 1-4 Z³ groups, and the other R³ is —NHR⁵

Embodiment 1(v) of this disclosure relates to any one of Embodiments 1, 1(a), 1(b), 1(c), 1(d), 1(e), 1(f) or 1(g), wherein one R³ is heteroarylalkyl optionally substituted with 1-4 Z³ groups, and the other R³ is —NHR⁵

Embodiment 1(w) of this disclosure relates to any one of Embodiments 1, 1(a), 1(b), 1(c), 1(d), 1(e), 1(f) or 1(g), wherein one R³ is arylalkyl optionally substituted with 1-4 Z³ groups, and the other R³ is —NHR⁵

Embodiment 1(v) of this disclosure relates to any one of Embodiments 1, 1(a), 1(b), 1(c), 1(d), 1(e), 1(f) or 1(g), wherein one R³ is arylalkyl optionally substituted with 1-4 Z³ groups, and the other R³ is —NHR⁵

Embodiment 1(w) of this disclosure relates to any one of Embodiments 1, 1(c), 1(d), or 1(e), wherein each X¹ and X² are H.

Embodiment 2 of this disclosure relates Embodiment 1 having Formula (II):

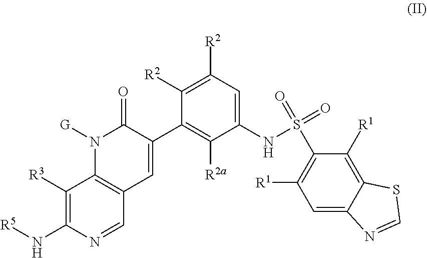

or a pharmaceutically acceptable salt, a tautomer, a stereoisomer, or a deuterated analog thereof, wherein:

G is H, C₁-C₆alkyl substituted with 0-4 X³ groups and 0-1 Z¹ group, —[C(X¹)(X²)]₀₋₄—C₃-C₆cycloalkyl substituted with 0-4 X⁴ groups and 0-1 Z¹ group, —[C(X¹)(X²)]₁_4-C₁-C₆alkoxy substituted with 0-3 X³ groups and 0-1 Z¹ group, or —[C(X¹)(X²)]₀₋₄-4-6 membered heterocycloalkyl substituted with 0-3 X⁴ groups and 0-1 Z¹ group;

each R¹ is independently H, halogen, CN, C₁-C₂alkyl optionally substituted with 1-3 Z² groups;

each R² is independently H, halogen, CN, C₁-C₂alkyl optionally substituted with 1-3 Z² groups;

R^{2a} is F or Cl;

R³ is H, CN, halogen, C₁-C₃alkyl optionally substituted with 1-3 Z² groups, C₁-C₃alkoxy optionally substituted with 1-3 Z² groups, or C₁-C₃alkoxyC₁-C₃alkyl optionally substituted with 1-3 Z² groups;

R⁵ is H, C₁-C₆alkyl optionally substituted with 1-4 Z² groups, C₁-C₄alkoxyC₁-C₄alkyl optionally substituted with 1-4 Z² groups, C₃-C₆cycloalkyl optionally substituted with 1-4 Z³ groups, —C₁-C₄alkyl-C₃-C₆cycloalkyl optionally substituted with 1-4 Z³ groups, 4-6 membered heterocycloalkyl optionally substituted with 1-4 Z³ groups, —C₁-C₄alkyl-4-6 membered heterocycloalkyl optionally substituted with 1-4 Z³ groups, 5-6 membered heteroaryl optionally substituted with 1-4 Z³ groups, —C₁-C₄alkyl-5-6 membered heteroaryl optionally substituted with 1-4 Z³ groups, phenyl optionally substituted with 1-4 Z³ groups, or —C₁-C₄alkyl-phenyl optionally substituted with 1-4 Z³ groups, provided that when R⁵ is a 4-6 membered heterocycloalkyl or 5-6 membered heteroaryl, then any heteroatom of R cannot be attached to the nitrogen atom of the group —NHR⁵;

each X¹ is independently H, halogen or methyl;

each X² is independently H, halogen or methyl;

each X³ is independently OH, CN or halogen;

each X⁴ is independently D, OH, CN, halogen, C₁-C₆alkyl optionally substituted with 1-4 Z² groups, or C₁-C₆alkoxy optionally substituted with 1-4 Z² groups;

Z¹ is cycloalkyl optionally substituted with 1-4 Z³ groups, cycloalkylalkyl optionally substituted with 1-4 Z³ groups, heterocycloalkyl optionally substituted with 1-4 Z³ groups, heterocycloalkylalkyl optionally substituted with 1-4 Z³ groups, heteroaryl optionally substituted with 1-4 Z³ groups, or heteroarylalkyl optionally substituted with 1-4 Z³ groups;

each Z² is independently halogen or OH; and

each Z³ is independently halogen, OH, or alkyl optionally substituted with 1-3 halogens, provided that when Z³ is halogen or OH, then Z³ cannot be attached to any heteroatom from R⁵.

### Subembodiments of Embodiment 2

Embodiment 2(a) of this disclosure relates to Embodiment 1, wherein G is H.

Embodiment 2(b) of this disclosure relates to Embodiment 1, wherein G is C₁-C₆alkyl substituted with 0-4 X³ groups and 0-1 Z¹ group.

Embodiment 2(c) of this disclosure relates to Embodiment 1, wherein G is —[C(X¹)(X²)]₀₋₄—C₃-C₆cycloalkyl substituted with 0-4 X⁴ groups and 0-1 Z¹ group.

Embodiment 2(d) of this disclosure relates to Embodiment 1, wherein G is —[C(X¹)(X²)]₁₋₄—C₁-C₆alkoxy substituted with 0-3 X³ groups and 0-1 Z¹ group.

Embodiment 2(e) of this disclosure relates to Embodiment 1, wherein G is —[C(X¹)(X²)]₀₋₄-4-6 membered heterocycloalkyl substituted with 0-3 X⁴ groups and 0-1 Z¹ group.

Embodiment 2(f) of this disclosure relates to any one of Embodiments 2, 2(a), 2(b), 2(c), 2(d), or 2(e), wherein each R¹ is independently H or halogen.

Embodiment 2(g) of this disclosure relates to any one of Embodiments 2, 2(a), 2(b), 2(c), 2(d), 2(e) or 2(f), wherein R², is F.

Embodiment 2(h) of this disclosure relates to any one of Embodiments 2, 2(a), 2(b), 2(c), 2(d), 2(e), 2(f) or 2(g), wherein R³ is H.

Embodiment 2(i) of this disclosure relates to any one of Embodiments 2, 2(a), 2(b), 2(c), 2(d), 2(e), 2(f) or 2(g), wherein R³ is CN.

Embodiment 2(j) of this disclosure relates to any one of Embodiments 2, 2(a), 2(b), 2(c), 2(d), 2(e), 2(f) or 2(g), wherein R³ is halogen.

Embodiment 2(k) of this disclosure relates to any one of Embodiments 2, 2(a), 2(b), 2(c), 2(d), 2(e), 2(f) or 2(g), wherein R³ is C₁-C₃alkyl optionally substituted with 1-3 Z² groups.

Embodiment 2(l) of this disclosure relates to any one of Embodiments 2, 2(a), 2(b), 2(c), 2(d), 2(e), 2(f) or 2(g), wherein R³ is C₁-C₃alkoxy optionally substituted with 1-3 Z² groups.

Embodiment 2(m) of this disclosure relates to any one of Embodiments 2, 2(a), 2(b), 2(c), 2(d), 2(e), 2(f) or 2(g), wherein R³ is C₁-C₃alkoxyC₁-C₃alkyl optionally substituted with 1-3 Z² groups.

Embodiment 2(n) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is H.

Embodiment 2(o) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is C₁-C₆alkyl optionally substituted with 1-4 Z² groups.

Embodiment 2(p) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is C₁-C₄alkoxyC₁-C₄alkyl optionally substituted with 1-4 Z² groups.

Embodiment 2(q) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is C₃-C₆cycloalkyl optionally substituted with 1-4 Z³ groups.

Embodiment 2(r) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is —C₁-C₄alkyl-C₃-C₆cycloalkyl optionally substituted with 1-4 Z³ groups.

Embodiment 2(s) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is a 4-6 membered heterocycloalkyl optionally substituted with 1-4 Z³ groups, provided that any heteroatom of R⁵ cannot be attached to the nitrogen atom of —NHR⁵.

Embodiment 2(t) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is C₁-C₄alkyl-4-6 membered heterocycloalkyl optionally substituted with 1-4 Z³ groups.

Embodiment 2(u) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is a 5-6 membered heteroaryl optionally substituted with 1-4 Z³ provided that any heteroatom of R⁵ cannot be attached to the nitrogen atom of —NHR⁵.

Embodiment 2(v) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is —C₁-C₄alkyl-5-6 membered heteroaryl optionally substituted with 1-4 Z³ groups.

Embodiment 2(w) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is phenyl optionally substituted with 1-4 Z³ groups.

Embodiment 2(x) of this disclosure relates to any one of Embodiments 2, 2(a), 2(b), 2(c), 2(d), 2(e), 2(f), 2(g), 2(h), 2(i), 2(j), 2(k), 2(l), or 2(m), wherein R⁵ is —C₁-C₄alkyl-phenyl optionally substituted with 1-4 Z³ groups.

Embodiment 2(y) of this disclosure relates to any one of Embodiments 2, 2(a), 2(b), 2(c), 2(d), 2(e), 2(f), 2(g), 2(h), 2(i), 2(j), 2(k), 2(l), 2(m), 2(n), 2(o), 2(p), 2(q), 2(r), 2(s), 2(t), 2(u), 2(v), 2(w) or 2(x), wherein each X¹ and X² are H.

Embodiment 3 of this disclosure relates to a compound having Formula (III):

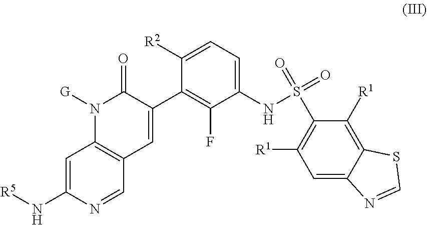

or a pharmaceutically acceptable salt, a tautomer, a stereoisomer, or a deuterated analog thereof, wherein:

G is H, C₁-C₄alkyl optionally substituted with 1-3 X³ groups, —[C(X¹)(X²)]₀₋₂—C₃-C₄cycloalkyl optionally substituted with 1-3 X⁴ groups, or C₁-C₄alkoxy optionally substituted with 1-3 X³ groups;

each R¹ is independently H, F or Cl;

R² is H, F or Cl;

R⁵ is H, C₁-C₆alkyl optionally substituted with 1-3 Z² groups, C₁-C₃alkoxyC₁-C₃alkyl optionally substituted with 1-3 Z² groups, C₃-C₄cycloalkyl optionally substituted with 1-3 Z³ groups, —C₁-C₃alkyl-C₃-C₄cycloalkyl optionally substituted with 1-3 Z³ groups, 4-6 membered heterocycloalkyl optionally substituted with 1-3 Z³ groups, —C₁-C₂alkyl-4-6 membered heterocycloalkyl optionally substituted with 1-3 Z³ groups, 5-6 membered heteroaryl optionally substituted with 1-3 Z³ groups, —C₁-C₂alkyl-5-6 membered heteroaryl optionally substituted with 1-3 Z³ groups, phenyl optionally substituted with 1-3 Z³ groups, or —C₁-C₂alkyl-phenyl optionally substituted with 1-3 Z³ groups, provided that when R⁵ is a 4-6 membered heterocycloalkyl or 5-6 membered heteroaryl, then any heteroatom of R cannot be attached to the nitrogen atom of —NHR⁵;

each X¹ is independently H, F, Cl, or CH₃;

each X² is independently H, F, Cl, or CH₃;

each X³ is independently F, Cl, or OH;

each X⁴ is independently F, Cl, OH or methyl;

each Z² is independently F, Cl or OH; and

each Z³ is independently F, Cl, OH, or C₁-C₃alkyl optionally substituted with 1-3 halogens, provided that when Z³ is F, Cl, or OH, then Z³ cannot be attached to any heteroatom from R⁵.

### Subembodiments of Embodiment 3

Embodiment 3(a) of this disclosure relates to Embodiment 3, wherein G is H.

Embodiment 3(b) of this disclosure relates to Embodiment 3, wherein G is C₁-C₄alkyl optionally substituted with 1-3 X³ groups.

Embodiment 3(c) of this disclosure relates to Embodiment 3, wherein G is —[C(X¹)(X²)]₀₋₂—C₃-C₄cycloalkyl optionally substituted with 1-3 X⁴ groups.

Embodiment 3(d) of this disclosure relates to Embodiment 3, wherein G is or C₁-C₄alkoxy optionally substituted with 1-3 X³ groups.

Embodiment 3(e) of this disclosure relates to any one of Embodiments 3, 3(a), 3(b), 3(c) or 3(d), wherein R² is H.

Embodiment 3(f) of this disclosure relates to any one of Embodiments 3, 3(a), 3(b), 3(c) or 3(d), wherein R² is F.

Embodiment 3(g) of this disclosure relates to any one of Embodiments 3, 3(a), 3(b), 3(c) or 3(d), wherein R² is Cl.

Embodiment 3(h) of this disclosure relates to any one of Embodiments 3, 3(a), 3(b), 3(c), 3(d), 3(e), 3(f), or 3(g), wherein R⁵ is H.

Embodiment 3(i) of this disclosure relates to any one of Embodiments 3, 3(a), 3(b), 3(c), 3(d), 3(e), 3(f), or 3(g), wherein R⁵ is C₁-C₆alkyl optionally substituted with 1-3 Z² groups.

Embodiment 3(j) of this disclosure relates to any one of Embodiments 3, 3(a), 3(b), 3(c), 3(d), 3(e), 3(f), or 3(g), wherein R⁵ is C₁-C₃alkoxyC₁-C₃alkyl optionally substituted with 1-3 Z² groups.

Embodiment 3(k) of this disclosure relates to any one of Embodiments 3, 3(a), 3(b), 3(c), 3(d), 3(e), 3(f), or 3(g), wherein R⁵ is C₃-C₄cycloalkyl optionally substituted with 1-3 Z³ groups.

Embodiment 3(l) of this disclosure relates to any one of Embodiments 3, 3(a), 3(b), 3(c), 3(d), 3(e), 3(f), or 3(g), wherein R⁵ is —C₁-C₃alkyl-C₃-C₄cycloalkyl optionally substituted with 1-3 Z³ groups.

Embodiment 3(m) of this disclosure relates to any one of Embodiments 3, 3(a), 3(b), 3(c), 3(d), 3(e), 3(f), or 3(g), wherein R⁵ is a 4-6 membered heterocycloalkyl optionally substituted with 1-3 Z³ groups provided that any heteroatom of R⁵ cannot be attached to the nitrogen atom of —NHR⁵.

Embodiment 3(n) of this disclosure relates to any one of Embodiments 3, 3(a), 3(b), 3(c), 3(d), 3(e), 3(f), or 3(g), wherein R⁵ is —C₁-C₂alkyl-4-6 membered heterocycloalkyl optionally substituted with 1-3 Z³ groups.

Embodiment 3(o) of this disclosure relates to any one of Embodiments 3, 3(a), 3(b), 3(c), 3(d), 3(e), 3(f), or 3(g), wherein R⁵ is a 5-6 membered heteroaryl optionally substituted with 1-3 Z³ groups provided that any heteroatom of R cannot be attached to the nitrogen atom of —NHR⁵.

Embodiment 3(p) of this disclosure relates to any one of Embodiments 3, 3(a), 3(b), 3(c), 3(d), 3(e), 3(f), or 3(g), wherein R⁵ is —C₁-C₂alkyl-5-6 membered heteroaryl optionally substituted with 1-3 Z³ groups.

Embodiment 3(q) of this disclosure relates to any one of Embodiments 3, 3(a), 3(b), 3(c), 3(d), 3(e), 3(f), or 3(g), wherein R⁵ is phenyl optionally substituted with 1-3 Z³ groups.

Embodiment 3(r) of this disclosure relates to any one of Embodiments 3, 3(a), 3(b), 3(c), 3(d), 3(e), 3(f), or 3(g), wherein R⁵ is —C₁-C₂alkyl-phenyl optionally substituted with 1-3 Z³ groups.

Embodiment 3(s) of this disclosure relates to any one of Embodiments 3, 3(a), 3(b), 3(c), 3(d), 3(e), 3(f), 3(g), 3(h), 3(i), 3(j), 3(k), 3(l), 3(m), 3(n), 3(o), 3(p), 3(q) or 3(r), wherein each Xi and X² are H.

Embodiment 4 of this disclosure relates to Embodiment 1 having one of the following Formulae:

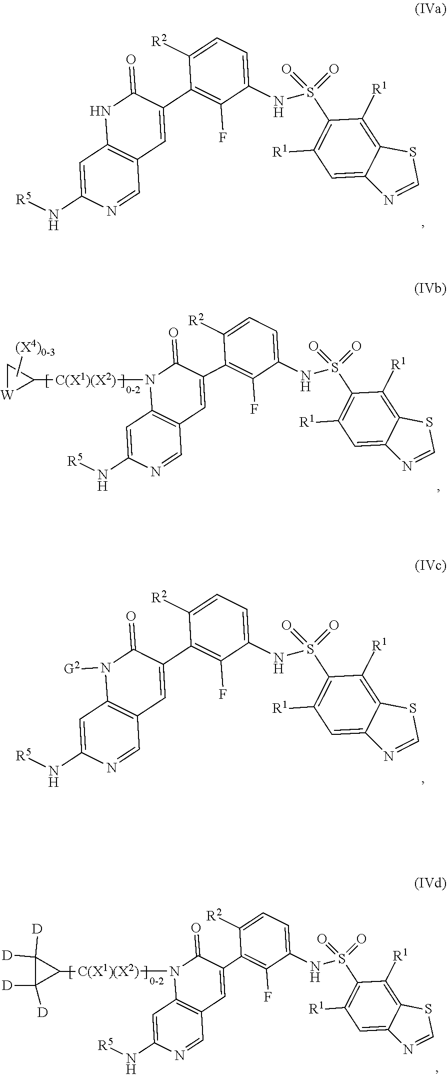

or a pharmaceutically acceptable salt, or a stereoisomer thereof, wherein:

G² is C₁-C₄alkyl optionally substituted with 1-3 X³ groups;

each R¹ is independently H, F or Cl;

R² is H, F or Cl;

R⁵ is H, C₁-C₆alkyl optionally substituted with 1-3 Z² groups, C₁-C₃alkoxyC₁-C₃alkyl optionally substituted with 1-3 Z² groups, C₃-C₄cycloalkyl optionally substituted with 1-3 Z³ groups, —C₁-C₃alkyl-C₃-C₄cycloalkyl optionally substituted with 1-3 Z³ groups, 4-6 membered heterocycloalkyl optionally substituted with 1-3 Z³ groups, —C₁-C₂alkyl-4-6 membered heterocycloalkyl optionally substituted with 1-3 Z³ groups, 5-6 membered heteroaryl optionally substituted with 1-3 Z³ groups, —C₁-C₂alkyl-5-6 membered heteroaryl optionally substituted with 1-3 Z³ groups, phenyl optionally substituted with 1-3 Z³ groups, or —C₁-C₂alkyl-phenyl optionally substituted with 1-3 Z³ groups, provided that when R⁵ is a 4-6 membered heterocycloalkyl or 5-6 membered heteroaryl, then any heteroatom of R cannot be attached to the nitrogen atom of —NHR⁵;

W is —CH₂—, —CH₂CH₂—, or —CH₂CH₂CH₂—;

each X¹ is independently H or F or CH₃;

each X² is independently H, F, or CH₃;

each X³ is independently F, Cl, or OH;

each X⁴ is independently F, Cl, OH or methyl;

each Z² is independently F, Cl or OH; and

each Z³ is independently F, Cl, OH, or C₁-C₃alkyl optionally substituted with 1-3 halogens, provided that when Z³ is F, Cl, or OH, then Z³ cannot be attached to any heteroatom from R⁵.

### Subembodiments of Embodiment 4

Embodiment 4(a) of this disclosure relates to Embodiment 4, wherein R² is H.

Embodiment 4(b) of this disclosure relates to Embodiment 4, wherein R² is F.

Embodiment 4(c) of this disclosure relates to Embodiment 4, wherein R² is Cl.

Embodiment 4(d) of this disclosure relates to Embodiment 4, 4(a), 4(b) or 4(c), wherein R⁵ is H.

Embodiment 4(e) of this disclosure relates to Embodiment 4, 4(a), 4(b) or 4(c), wherein R⁵ is C₁-C₆alkyl optionally substituted with 1-3 Z² groups.

Embodiment 4(f) of this disclosure relates to Embodiment 4, 4(a), 4(b) or 4(c), wherein R⁵ is C₁-C₃alkoxyC₁-C₃alkyl optionally substituted with 1-3 Z² groups.

Embodiment 4(g) of this disclosure relates to Embodiment 4, 4(a), 4(b) or 4(c), wherein R⁵ is C₃-C₄cycloalkyl optionally substituted with 1-3 Z³ groups.

Embodiment 4(h) of this disclosure relates to Embodiment 4, 4(a), 4(b) or 4(c), wherein R⁵ is —C₁-C₃alkyl-C₃-C₄cycloalkyl optionally substituted with 1-3 Z³ groups.

Embodiment 4(i) of this disclosure relates to Embodiment 4, 4(a), 4(b) or 4(c), wherein R⁵ is 4-6 membered heterocycloalkyl optionally substituted with 1-3 Z³ groups, provided that any heteroatom of R⁵ cannot be attached to the nitrogen atom of —NHR⁵.

Embodiment 4(j) of this disclosure relates to Embodiment 4, 4(a), 4(b) or 4(c), wherein R⁵ is —C₁-C₂alkyl-4-6 membered heterocycloalkyl optionally substituted with 1-3 Z³ groups.

Embodiment 4(k) of this disclosure relates to Embodiment 4, 4(a), 4(b) or 4(c), wherein R⁵ is a 5-6 membered heteroaryl optionally substituted with 1-3 Z³ groups, provided that any heteroatom of R⁵ cannot be attached to the nitrogen atom of —NHR⁵.

Embodiment 4(l) of this disclosure relates to Embodiment 4, 4(a), 4(b) or 4(c),

Embodiment 4(m) of this disclosure relates to Embodiment 4, 4(a), 4(b) or 4(c), wherein R⁵ is —C₁-C₂alkyl-5-6 membered heteroaryl optionally substituted with 1-3 Z³ groups.

Embodiment 4(n) of this disclosure relates to Embodiment 4, 4(a), 4(b) or 4(c), wherein R⁵ is phenyl optionally substituted with 1-3 Z³ groups.

Embodiment 4(o) of this disclosure relates to Embodiment 4, 4(a), 4(b) or 4(c), wherein R⁵ is or —C₁-C₂alkyl-phenyl optionally substituted with 1-3 Z³ groups.

Embodiment 4(p) of this disclosure relates to any one of Embodiments 4, 4(a), 4(b), 4(c), 4(d), 4(e), 4(f), 4(g), 4(h), 4(i), 4(j), 4(k), 4(l), 4(m), 4(n) or 4(o), wherein each X¹ and X² are H.

Embodiment 5 of this disclosure relates to Embodiment 4 having Formula (IVa), wherein:

each R¹ is independently H or F;

R² is H or F; and

R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R⁵ is a 5-6 membered heteroaryl, then any heteroatom of the 5-6 membered heteroaryl cannot be attached to the nitrogen atom of —NHR⁵.

### Subembodiments of Embodiment 5

Embodiment 5(a) of this disclosure relates to Embodiment 5, wherein R² is H.

Embodiment 5(b) of this disclosure relates to Embodiment 5, wherein R² is F.

Embodiment 5(c) of this disclosure relates to Embodiment 5, 5(a) or 5(b), wherein R⁵ is H.

Embodiment 5(d) of this disclosure relates to Embodiment 5, 5(a) or 5(b), wherein R⁵ is C₁-C₄alkyl optionally substituted with 1-3 Z² groups.

Embodiment 5(e) of this disclosure relates to Embodiment 5, 5(a) or 5(b), wherein R⁵ is C₁-C₃alkoxyC₁-C₃alkyl optionally substituted with 1-3 Z² groups.

Embodiment 5(f) of this disclosure relates to Embodiment 5, 5(a) or 5(b), wherein R⁵ is a 5-6 membered heteroaryl optionally substituted with 1-3 Z³ groups, provided that any heteroatom of the 5-6 membered heteroaryl cannot be attached to the nitrogen atom of —NHR⁵.

Embodiment 5(g) of this disclosure relates to Embodiment 5, 5(a) or 5(b), wherein R⁵ is —C₁-C₂alkyl-5-6 membered heteroaryl optionally substituted with 1-3 Z³ groups.

Embodiment 6 of this disclosure relates to Embodiment 4 having Formula (IVb), wherein:

each R¹ is independently H or F;

R² is H or F;

R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R⁵ is a 5-6 membered heteroaryl, then any heteroatom of the 5-6 membered heteroaryl cannot be attached to the nitrogen atom of —NHR⁵;

W is —CH₂— or —CH₂CH₂—;

each X² is independently H or F; and

each X⁴ is independently F or methyl.

### Subembodiments of Embodiment 6

Embodiment 6(a) of this disclosure relates to Embodiment 6, wherein R² is H.

Embodiment 6(b) of this disclosure relates to Embodiment 6, wherein R² is F.

Embodiment 6(c) of this disclosure relates to Embodiment 6, wherein R⁵ is H.

Embodiment 6(d) of this disclosure relates to Embodiment 6, 6(a) or 6(b), wherein R⁵ is C₁-C₄alkyl optionally substituted with 1-3 Z² groups

Embodiment 6(e) of this disclosure relates to Embodiment 6, 6(a) or 6(b), wherein R⁵ is C₁-C₃alkoxyC₁-C₃alkyl optionally substituted with 1-3 Z² groups.

Embodiment 6(f) of this disclosure relates to Embodiment 6, 6(a) or 6(b), wherein R⁵ is a 5-6 membered heteroaryl optionally substituted with 1-3 Z³ groups, provided that any heteroatom of the 5-6 membered heteroaryl cannot be attached to the nitrogen atom of —NHR⁵.

Embodiment 6(g) of this disclosure relates to Embodiment 6, 6(a) or 6(b), wherein R⁵ is —C₁-C₂alkyl-5-6 membered heteroaryl optionally substituted with 1-3 Z³ groups.

Embodiment 6(h) of this disclosure relates to Embodiment 6, 6(a), 6(c), 6(c), 6(d), 6(e), 6(f) or 6(g), wherein W is —CH₂-.

Embodiment 6(i) of this disclosure relates to Embodiment 6, 6(a), 6(c), 6(c), 6(d), 6(e), 6(f) or 6(g), wherein W is —CH₂CH₂-.

Embodiment 6(j) of this disclosure relates to Embodiment 6, 6(a), 6(c), 6(c), 6(d), 6(e), 6(f) or 6(g), wherein W is —CH₂CH₂CH₂-.

Embodiment 6(k) of this disclosure relates to any one of Embodiments 6, 6(a), 6(b), 6(c), 6(d), 6(e), 6(f) or 6(g), wherein each X¹ and X² are H.

Embodiment 7 of this disclosure relates to Embodiment 4 having Formula (IVc), wherein:

G² is C₁-C₄alkyl optionally substituted with 1-3 F;

R² is H or F; and

R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R is a 5-6 membered heteroaryl, then any heteroatom of the 5-6 membered heteroaryl cannot be attached to the nitrogen atom of —NHR⁵.

### Subembodiments of Embodiment 7

Embodiment 7(a) of this disclosure relates to Embodiment 7, wherein R² is H.

Embodiment 7(b) of this disclosure relates to Embodiment 7, wherein R² is F.

Embodiment 7(c) of this disclosure relates to Embodiment 7, 7(a) or 7(b), wherein R⁵ is H.

Embodiment 7(d) of this disclosure relates to Embodiment 7, 7(a) or 7(b), wherein R⁵ is C₁-C₄alkyl optionally substituted with 1-3 Z² groups.

Embodiment 7(e) of this disclosure relates to Embodiment 7, 7(a) or 7(b), wherein R⁵ is C₁-C₃alkoxyC₁-C₃alkyl optionally substituted with 1-3 Z² groups.

Embodiment 7(f) of this disclosure relates to Embodiment 7, 7(a) or 7(b), wherein R⁵ is a 5-6 membered heteroaryl optionally substituted with 1-3 Z³ groups provided that any heteroatom of the 5-6 membered heteroaryl cannot be attached to the nitrogen atom of —NHR⁵.

Embodiment 7(g) of this disclosure relates to Embodiment 7, 7(a) or 7(b), wherein R⁵ is —C₁-C₂alkyl-5-6 membered heteroaryl optionally substituted with 1-3 Z³ groups.

Embodiment 8 of this disclosure relates to Embodiment 4 having Formula (IVd), wherein:

each R¹ is independently H or F;

R² is H or F;

R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R⁵ is a 5-6 membered heteroaryl, then any heteroatom of the 5-6 membered heteroaryl cannot be attached to the nitrogen atom of —NHR⁵;

W is —CH₂— or —CH₂CH₂—; and

each X² is independently H or F.

### Subembodiments of Embodiment 8

Embodiment 8(a) of this disclosure relates to Embodiment 8, wherein R² is H.

Embodiment 8(b) of this disclosure relates to Embodiment 8, wherein R² is F.

Embodiment 8(c) of this disclosure relates to Embodiment 8, 8(a) or 8(b), wherein R⁵ is H.

Embodiment 8(d) of this disclosure relates to Embodiment 8, 8(a) or 8(b), wherein R⁵ is C₁-C₄alkyl optionally substituted with 1-3 Z² groups.

Embodiment 8(e) of this disclosure relates to Embodiment 8, 8(a) or 8(b), wherein R⁵ is C₁-C₃alkoxyC₁-C₃alkyl optionally substituted with 1-3 Z² groups.

Embodiment 8(f) of this disclosure relates to Embodiment 8, 8(a) or 8(b), wherein R⁵ is a 5-6 membered heteroaryl optionally substituted with 1-3 Z³ groups provided that any heteroatom of the 5-6 membered heteroaryl cannot be attached to the nitrogen atom of —NHR⁵.

Embodiment 8(g) of this disclosure relates to Embodiment 8, 8(a) or 8(b), wherein R⁵ is —C₁-C₂alkyl-5-6 membered heteroaryl optionally substituted with 1-3 Z³ groups.

Embodiment 8(h) of this disclosure relates to any one of Embodiments 8, 8(a), 8(b), 8(c), 8(d), 8(e), 8(f) or 8(g), wherein each X¹ and X² are H.

Embodiment 9 of this disclosure relates to Embodiment 1 having one of the following Formulae:

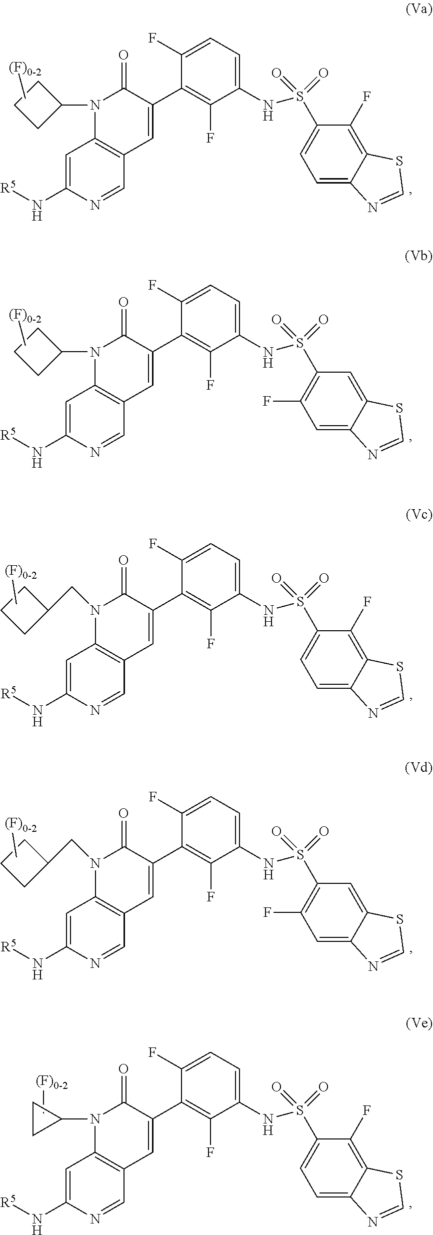

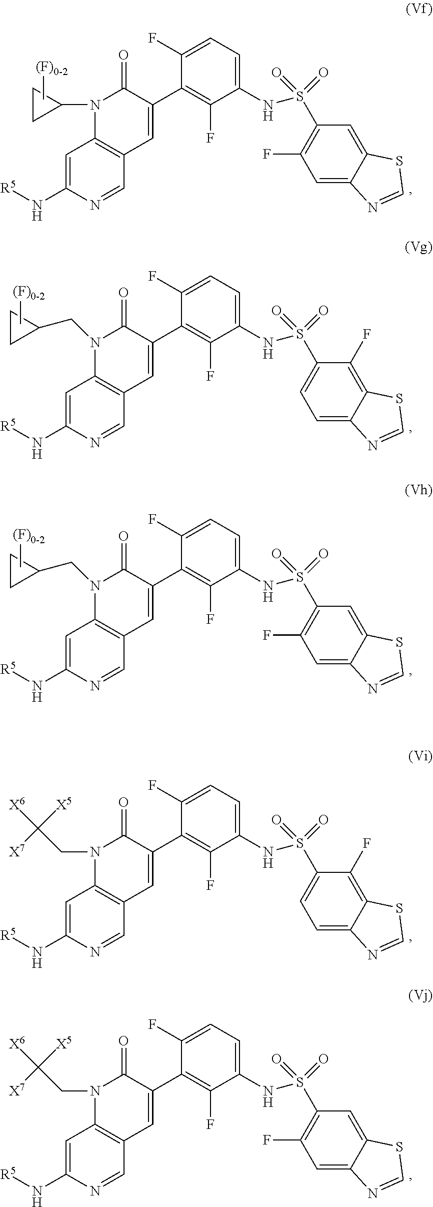

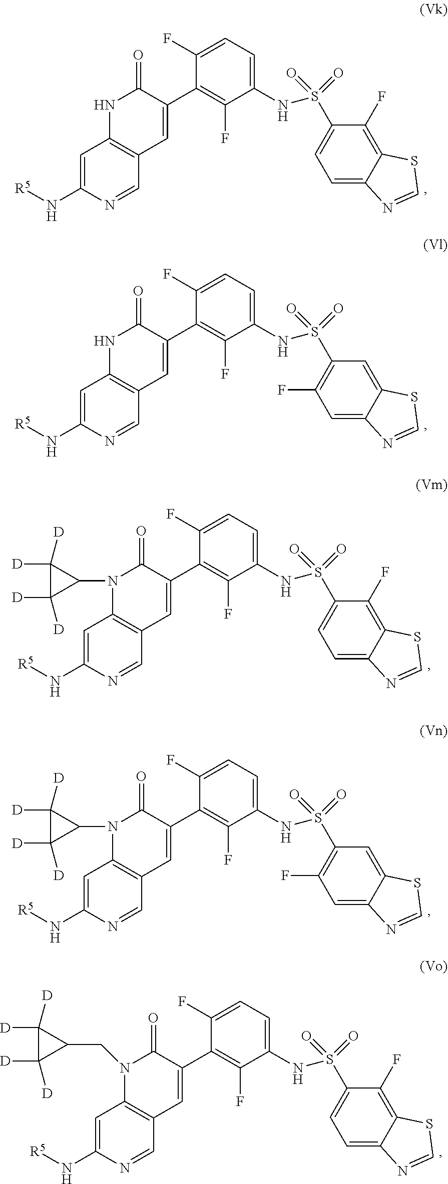

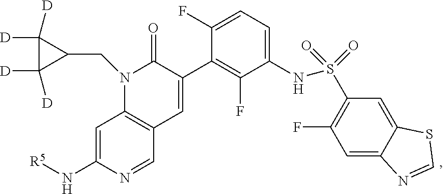

or a pharmaceutically acceptable salt or a stereoisomer thereof, wherein.

R⁵ is H, C₁-C₆alkyl optionally substituted with 1-3 Z² groups, C₁-C₃alkoxyC₁-C₃alkyl optionally substituted with 1-3 Z² groups, C₃-C₄cycloalkyl optionally substituted with 1-3 Z³ groups, —C₁-C₃alkyl-C₃-C₄cycloalkyl optionally substituted with 1-3 Z³ groups, 4-6 membered heterocycloalkyl optionally substituted with 1-3 Z³ groups, —C₁-C₂alkyl-4-6 membered heterocycloalkyl optionally substituted with 1-3 Z³ groups, 5-6 membered heteroaryl optionally substituted with 1-3 Z³ groups, —C₁-C₂alkyl-5-6 membered heteroaryl optionally substituted with 1-3 Z³ groups, phenyl optionally substituted with 1-3 Z³ groups, or —C₁-C₂alkyl-phenyl optionally substituted with 1-3 Z³ groups, provided that when R⁵ is a 4-6 membered heterocycloalkyl or 5-6 membered heteroaryl, then any heteroatom of R⁵ cannot be attached to the nitrogen atom of —NHR⁵;

X⁵ is H or F;

X⁶ is H, F, or CH₃;

X⁷ is H, F, or CH₃;

each Z² is independently F, Cl or OH; and

each Z³ is independently F, Cl, OH, or C₁-C₃alkyl optionally substituted with 1-3 halogens, provided that when Z³ is F, Cl, or OH, then Z³ cannot be attached to any heteroatom from R.

### Subembodiments of Embodiment 9

Embodiment 9(a) of this disclosure relates to Embodiment 9, wherein R⁵ is H.

Embodiment 9(b) of this disclosure relates to Embodiment 9, wherein R⁵ is C₁-C₆alkyl optionally substituted with 1-3 Z² groups.

Embodiment 9(b) of this disclosure relates to Embodiment 9, wherein R⁵ is C₁-C₃alkoxyC₁-C₃alkyl optionally substituted with 1-3 Z² groups.

Embodiment 9(c) of this disclosure relates to Embodiment 9, wherein R⁵ is C₃-C₄cycloalkyl optionally substituted with 1-3 Z³ groups.

Embodiment 9(d) of this disclosure relates to Embodiment 9, wherein R⁵ is —C₁-C₃alkyl-C₃-C₄cycloalkyl optionally substituted with 1-3 Z³ groups.

Embodiment 9(e) of this disclosure relates to Embodiment 9, wherein R⁵ is a 4-6 membered heterocycloalkyl optionally substituted with 1-3 Z³ groups, provided that any heteroatom of R cannot be attached to the nitrogen atom of —NHR⁵.

Embodiment 9(f) of this disclosure relates to Embodiment 9, wherein R⁵ is —C₁-C₂alkyl-4-6 membered heterocycloalkyl optionally substituted with 1-3 Z³ groups.

Embodiment 9(g) of this disclosure relates to Embodiment 9, wherein R⁵ is a 5-6 membered heteroaryl optionally substituted with 1-3 Z³ groups, provided that any heteroatom of R⁵cannot be attached to the nitrogen atom of —NHR⁵.

Embodiment 9(h) of this disclosure relates to Embodiment 9, wherein R⁵ is —C₁-C₂alkyl-5-6 membered heteroaryl optionally substituted with 1-3 Z³ groups.

Embodiment 9(i) of this disclosure relates to Embodiment 9, wherein R⁵ is phenyl optionally substituted with 1-3 Z³ groups.

Embodiment 9(j) of this disclosure relates to Embodiment 9, wherein R⁵ is or —C₁-C₂alkyl-phenyl optionally substituted with 1-3 Z³ groups.

Embodiment 10 of this disclosure relates to Embodiment 9 having one of Formulae (Va), (Vb), (Vc), (Vd), or a pharmaceutically acceptable salt, a tautomer, a stereoisomer, or a deuterated analog thereof.

Embodiment 11 of this disclosure relates to Embodiment 9 having one of Formulae (Ve), (Vf), (Vg), (Vh), or a pharmaceutically acceptable salt, a tautomer, a stereoisomer, or a deuterated analog thereof.

Embodiment 12 of this disclosure relates to Embodiment 9 having one of Formulae (Vi), (Vj), or a pharmaceutically acceptable salt, a tautomer, a stereoisomer, or a deuterated analog thereof.

Embodiment 13 of this disclosure relates to Embodiment 9 having one of Formulae (Vk), (Vl), or a pharmaceutically acceptable salt, a tautomer, a stereoisomer, or a deuterated analog thereof.

Embodiment 14 of this disclosure relates to any of Embodiments 1-13, wherein R⁵ is H, CH₃, hydroxyethyl, methoxyethyl, or 1-(difluoromethyl)-1H-pyrazolyl, provided that a nitrogen atom of 1-(difluoromethyl)-1H-pyrazolyl cannot be attached to the nitrogen atom of —NHR⁵.

Embodiment 15 of this disclosure relates to a compound selected from Table 1, or a pharmaceutically acceptable salt, thereof.

In other embodiments of the compounds described herein, one or more of the compounds described in any of Embodiments 1-15, including any sub-embodiments thereof, has a selectivity of CSK over LCK of at least 10, 20, 50, 100, 200, 300, 400 or 500 fold greater.

Compounds contemplated herein are described with reference to both generic formulae and specific compounds. In addition, the compounds described herein may exist in a number of different forms or derivatives, all within the scope of the present disclosure. These include, for example, tautomers, stereoisomers, racemic mixtures, regioisomers, salts, prodrugs (e.g. carboxylic acid esters), and active metabolites.

It is understood that some compounds may exhibit tautomerism. In such cases, the formulae provided herein expressly depict only one of the possible tautomeric forms. It is therefore to be understood that the formulae provided herein are intended to represent any tautomeric form of the depicted compounds and are not to be limited merely to the specific tautomeric form depicted by the drawings of the formulae.

Likewise, some of the compounds according to the present disclosure may exist as stereoisomers as defined herein. All such single stereoisomers, racemates and mixtures thereof are intended to be within the scope of the present disclosure. Unless specified to the contrary, all such stereoisomeric forms are included within the formulae provided herein.

In some embodiments, a chiral compound of the present disclosure is in a form that contains at least 80% of a single isomer (60% enantiomeric excess (“e.e.”) or diastereomeric excess (“d.e.”)), or at least 85% (70% e.e. or d.e.), 90% (80% e.e. or d.e.), 95% (90% e.e. or d.e.), 97.5% (95% e.e. or d.e.), or 99% (98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In some embodiments, the compound is present in optically pure form.

For compounds in which synthesis involves addition of a single group at a double bond, particularly a carbon-carbon double bond, the addition may occur at either of the double bond-linked atoms. For such compounds, the present disclosure includes both such regioisomers.

In addition to the present formulae and compounds described herein, the disclosure also includes prodrugs (generally pharmaceutically acceptable prodrugs), active metabolic derivatives (active metabolites), and their pharmaceutically acceptable salts.

Unless specified to the contrary, specification of a compound herein includes pharmaceutically acceptable salts of such compound.

In some embodiments, compounds of the disclosure are complexed with an acid or a base, including base addition salts such as ammonium, diethylamine, ethanolamine, ethylenediamine, diethanolamine, t-butylamine, piperazine, meglumine; acid addition salts, such as acetate, acetylsalicylate, besylate, camsylate, citrate, formate, fumarate, glutarate, hydrochlorate, maleate, mesylate, nitrate, oxalate, phosphate, succinate, sulfate, tartrate, thiocyanate and tosylate; and amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. In some instances, the amorphous form of the complex is facilitated by additional processing, such as by spray-drying, mechanochemical methods such as roller compaction, or microwave irradiation of the parent compound mixed with the acid or base. Such methods may also include addition of ionic and/or non-ionic polymer systems, including, but not limited to, hydroxypropyl methyl cellulose acetate succinate (HPMCAS) and methacrylic acid copolymer (e.g. Eudragit® L100-55), that further stabilize the amorphous nature of the complex. Such amorphous complexes provide several advantages. For example, lowering of the melting temperature relative to the free base facilitates additional processing, such as hot melt extrusion, to further improve the biopharmaceutical properties of the compound. Also, the amorphous complex is readily friable, which provides improved compression for loading of the solid into capsule or tablet form.

### III. Formulations and Administration

Embodiment 16 of this disclosure relates to a pharmaceutical composition comprising a compound in one of Embodiments 1-15, or any of the subembodiments thereof, and a pharmaceutically acceptable carrier.

Embodiment 17 of this disclosure relates to the pharmaceutical composition of Embodiment 16, further comprising a second pharmaceutical agent.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in The Science and Practice of Pharmacy, 21^{st} edition, Lippincott, Williams and Wilkins, Philadelphia, Pa., 2005 (hereby incorporated by reference herein).

Compounds of the present disclosure (i.e. any of the compounds described in Embodiments 1-15, including any of the subembodiments thereof) can be formulated as pharmaceutically acceptable salts.

Carriers or excipients can be used to produce compositions. The carriers or excipients can be chosen to facilitate administration of the compound. Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

The compounds can be administered by different routes including intravenous, intraperitoneal, subcutaneous, intramuscular, oral, transmucosal, rectal, transdermal, or inhalant. In some embodiments, the compounds can be administered by oral administration. For oral administration, for example, the compounds can be formulated into conventional oral dosage forms such as capsules, tablets, and liquid preparations such as syrups, elixirs, and concentrated drops.

For inhalants, compounds of the disclosure may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The compounds of the disclosure may also be used in combination with other inhaled therapies, for example corticosteroids such as fluticasone propionate, beclomethasone dipropionate, triamcinolone acetonide, budesonide, and mometasone furoate; beta agonists such as albuterol, salmeterol, and formoterol; anticholinergic agents such as ipratropium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

Pharmaceutical preparations for oral use can be obtained, for example, by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone). If desired, disintegrating agents may be added, such as the cross-linked polyvinylpyrrolidone, agar, or alginic acid, or a salt thereof such as sodium alginate.

Dragee cores are provided with suitable coatings. For this purpose, concentrated sugar solutions may be used, which may optionally contain, for example, gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the compounds of the disclosure are formulated in sterile liquid solutions, such as in physiologically compatible buffers or solutions, such as saline solution, Hank's solution, or Ringer's solution. In addition, the compounds may be formulated in solid form and redissolved or suspended immediately prior to use. Lyophilized forms can also be produced.

Administration can also be by transmucosal, topical, transdermal, or inhalant means. For transmucosal, topical or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal).

The topical compositions of this disclosure are formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In another embodiment, the carriers are those in which the active ingredient is soluble. Emulsifiers, stabilizers, humectants and antioxidants may also be included as well as agents imparting color or fragrance, if desired. Creams for topical application are formulated from a mixture of mineral oil, self-emulsifying beeswax and water in which mixture the active ingredient, dissolved in a small amount solvent (e.g. an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will, of course, be continuous rather than intermittent throughout the dosage regimen.

The amounts of various compounds to be administered can be determined by standard procedures taking into account factors such as the compound IC₅₀, the biological half-life of the compound, the age, size, and weight of the subject, and the indication being treated. The importance of these and other factors are well known to those of ordinary skill in the art. Generally, a dose will be between about 0.01 and 50 mg/kg, or 0.1 and 20 mg/kg of the subject being treated. Multiple doses may be used.

The compounds of the disclosure may also be used in combination with other therapies for treating the same disease. Such combination use includes administration of the compounds and one or more other therapeutics at different times, or co-administration of the compound and one or more other therapies. In some embodiments, dosage may be modified for one or more of the compounds of the disclosure or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a compound of the present disclosure, or at the same time as a compound of the disclosure. Use in combination also includes use with a therapy or medical procedure that is administered once or infrequently, such as surgery, along with a compound of the disclosure administered within a short time or longer time before or after the other therapy or procedure. In some embodiments, the present disclosure provides for delivery of compounds of the disclosure and one or more other drug therapeutics delivered by a different route of administration or by the same route of administration. The use in combination for any route of administration includes delivery of compounds of the disclosure and one or more other drug therapeutics delivered by the same route of administration together in any formulation, including formulations where the two compounds are chemically linked in such a way that they maintain their therapeutic activity when administered. In one aspect, the other drug therapy may be co-administered with one or more compounds of the disclosure. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations of compounds of the disclosure and one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

### IV. Methods of Use

The methods and compounds will typically be used in therapy for human subjects. However, they may also be used to treat similar or identical indications in other animal subjects.

In certain embodiments, the patient is 60 years or older and relapsed after a first line cancer therapy. In certain embodiments, the patient is 18 years or older and is relapsed or refractory after a second line cancer therapy. In certain embodiments, the patient is 60 years or older and is primary refractory to a first line cancer therapy. In certain embodiments, the patient is 70 years or older and is previously untreated. In certain embodiments, the patient is 70 years or older and is ineligible and/or unlikely to benefit from cancer therapy.

In certain embodiments, the therapeutically effective amount used in the methods provided herein is at least 10 mg per day. In certain embodiments, the therapeutically effective amount is 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, or 2500 mg per day. In other embodiments, the therapeutically effective amount is 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500, 3000, 3500, 4000, 4500, or 5000 mg per day or more. In certain embodiments, the compound is administered continuously.

In certain embodiments, provided herein is a method for treating a diseases or condition mediated by CSK by administering to a mammal having a disease or condition at least 10, 50, 90, 100, 135, 150, 200, 250, 300, 350, 400, 450, 500, 600, 700, 800, 900, 1000, 1200, 1300, 1400, 1500, 1600, 1700, 1800, 1900, 2000, 2200, 2500, 3000, 3500, 4000, 4500, or 5000 mg per day of any of the compounds described in a compound in one of Embodiments 1-15, or a pharmaceutically acceptable salt, deuterated analog, a tautomer or a stereoisomer thereof, and wherein the compound is administered on an empty stomach.

Embodiment 18 of this disclosure relates to a method for treating a subject with a disease or condition mediated by CSK or T-cell activation, said method comprising administering to the subject an effective amount of a compound in one of Embodiments 1-15, or any of the subembodiments thereof, or a pharmaceutically acceptable salt, deuterated analog, a tautomer or a stereoisomer thereof, or a pharmaceutical composition in one of Embodiments 16-17.

Embodiment 19 of this disclosure relates to Embodiment 18, wherein there is selective inhibition of CSK over LCK.

Embodiment 20 of this disclosure relates to a method for treatment of a disease or condition according to Embodiment 18 or 19, wherein the disease or condition is a neoplastic disorder, a cancer, an age-related disease, an inflammatory disorder, a cognitive disorder and or a neurodegenerative disease.

Embodiment 21 of this disclosure relates to a method for treatment of a disease or condition according to Embodiment 18 or 19, wherein the disease or condition is colorectal cancer, ovarian cancer, breast cancer, lung cancer, liver cancer, prostate cancer, kidney cancer, lymphoma, melanoma, pancreatic cancer, or leiomyosarcoma, e.g. uterine leiomyosarcoma.

Embodiment 21(a) of this disclosure relates to a method for treatment of a disease or condition according to Embodiment 21, wherein the disease or condition is colorectal cancer, ovarian cancer, breast cancer, lung cancer, liver cancer, prostate cancer, kidney cancer, pancreatic cancer, or leiomyosarcoma, e.g. uterine leiomyosarcoma. In some embodiments of Embodiment 21 and Embodiment 21(a), the cancer is refractory to treatment with one or more other kinase inhibitors.

### V. Combination Therapy

CSK modulators may be usefully combined with another pharmacologically active compound, or with two or more other pharmacologically active compounds, particularly in the treatment of cancer. In one embodiment, the composition includes any one or more compound(s) as described herein along with one or more compounds that are therapeutically effective for the same disease indication, wherein the compounds have a synergistic effect on the disease indication. In one embodiment, the composition includes any one or more compound(s) as described herein effective in treating a cancer and one or more other compounds that are effective in treating the same cancer, further wherein the compounds are synergistically effective in treating the cancer.

Embodiment 22 of this disclosure relates the method according to any one of Embodiments 18-21, further comprising administering one or more additional therapeutic agents.

Embodiment 23 of this disclosure relates the method according to Embodiment 22, wherein the one or more additional therapeutic agents is one or more of i) an alkylating agent selected from adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, oxaliplatin, piposulfan, semustine, streptozocin, temozolomide, thiotepa, and treosulfan; ii) an antibiotic selected from bleomycin, dactinomycin, daunorubicin, doxorubicin, epirubicin, idarubicin, menogaril, mitomycin, mitoxantrone, neocarzinostatin, pentostatin, and plicamycin; iii) an antimetabolite selected from azacitidine, capecitabine, cladribine, clofarabine, cytarabine, decitabine, floxuridine, fludarabine, 5-fluorouracil, ftorafur, gemcitabine, hydroxyurea, mercaptopurine, methotrexate, nelarabine, pemetrexed, raltitrexed, thioguanine, and trimetrexate; iv) an immune checkpoint agent selected from a PD-1 inhibitor, a PD-L1 inhibitor, and an anti-CTLA4 inhibitor; v) a hormone or hormone antagonist selected from enzalutamide, abiraterone, anastrozole, androgens, buserelin, diethylstilbestrol, exemestane, flutamide, fulvestrant, goserelin, idoxifene, letrozole, leuprolide, magestrol, raloxifene, tamoxifen, and toremifene; vi) a taxane selected from DJ-927, docetaxel, TPI 287, paclitaxel and DHA-paclitaxel; vii) a retinoid selected from alitretinoin, bexarotene, fenretinide, isotretinoin, and tretinoin; viii) an alkaloid selected from etoposide, homoharringtonine, teniposide, vinblastine, vincristine, vindesine, and vinorelbine; ix) an antiangiogenic agent selected from AE-941 (GW786034, Neovastat), ABT-510, 2-methoxyestradiol, lenalidomide, and thalidomide; x) a topoisomerase inhibitor selected from amsacrine, edotecarin, exatecan, irinotecan, SN-38 (7-ethyl-10-hydroxy-camptothecin), rubitecan, topotecan, and 9-aminocamptothecin; xi) a kinase inhibitor selected from erlotinib, gefitinib, flavopiridol, imatinib mesylate, lapatinib, sorafenib, sunitinib malate, 7-hydroxystaurosporine, and vatalanib; xii) a targeted signal transduction inhibitor selected from bortezomib, geldanamycin, and rapamycin; xiii) a biological response modifier selected from imiquimod, interferon-α and interleukin-2; xiv) an IDO inhibitor; xv) a chemotherapeutic agent selected from 3-AP (3-amino-2-carboxyaldehyde thiosemicarbazone), altrasentan, aminoglutethimide, anagrelide, asparaginase, bryostatin-1, cilengitide, elesclomol, eribulin mesylate, ixabepilone, lonidamine, masoprocol, mitoguanazone, oblimersen, sulindac, testolactone, tiazofurin, an mTOR inhibitor, a PI3K inhibitor, a Cdk4 inhibitor, an Akt inhibitor, a Hsp90 inhibitor, a farnesyltransferase inhibitor and an aromatase inhibitor (anastrozole letrozole exemestane); xvi) a BRAF inhibitor e.g., vemurafenib, dabrafenib, or encorafenib; xvii) a Mek inhibitor e.g, cobimetinib, trametinib, binimetinib or selumetinib; xviii) a c-Kit mutant inhibitor, xix) an EGFR inhibitor, xx) an epigenetic modulator; xxi) other adenosine axis blockade agents selected from CD39, CD38, A2AR and A2BR; xxii) agonists of TNFA super family member; or xxiii) an anti-ErbB2 mAb.

In another embodiment, the present disclosure provides a method of treating a cancer in a subject in need thereof by administering to the subject an effective amount of a composition including any one or more compound(s) as described herein in combination with one or more other therapies or medical procedures effective in treating the cancer. Other therapies or medical procedures include suitable anticancer therapy (e.g. drug therapy, vaccine therapy, gene therapy, photodynamic therapy) or medical procedure (e.g. surgery, radiation treatment, hyperthermia heating, bone marrow or stem cell transplant). In one embodiment, the one or more suitable anticancer therapies or medical procedures is selected from treatment with a chemotherapeutic agent (e.g. chemotherapeutic drug), radiation treatment (e.g. x-ray, gamma-ray, or electron, proton, neutron, or alpha-particle beam), hyperthermia heating (e.g. microwave, ultrasound, radiofrequency ablation), Vaccine therapy (e.g. AFP gene hepatocellular carcinoma vaccine, AFP adenoviral vector vaccine, AG-858, allogeneic GM-CSF-secretion breast cancer vaccine, dendritic cell peptide vaccines), gene therapy (e.g. Ad5CMV-p53 vector, adenovector encoding MDA7, adenovirus 5-tumor necrosis factor alpha), photodynamic therapy (e.g. aminolevulinic acid, motexatin lutetium), surgery, or bone marrow and stem cell transplantation.

It has been observed that CSK inhibitors described herein can exhibit off-target MAP2K4 inhibitory activity, as verified by crystal structural studies using a surrogate of MAP2K4 (MAP2K1). One of the most frequent alterations for leiomyosarcomas is in MAP2K4. Uterine leiomyosarcomas (LMS) arises from the myometrium, the smooth muscle layer of the uterus. They represent the most common type of uterine sarcomas, consisting of up to 80% of uterine sarcomas.

### VI. Kits

In another aspect, the present disclosure provides kits that include one or more compounds as described in any one of a compound in one of Embodiments 1-15, or a pharmaceutically acceptable salt, deuterated analog, a tautomer or a stereoisomer thereof, or a pharmaceutical composition in one of Embodiments 16-17. In some embodiments, the compound or composition is packaged, e.g., in a vial, bottle, flask, which may be further packaged, e.g., within a box, envelope, or bag. The compound or composition may be approved by the U.S. Food and Drug Administration or similar regulatory agency for administration to a mammal, e.g., a human. The compound or composition may be approved for administration to a mammal, e.g., a human, for a CSK mediated disease or condition. The kits described herein may include written instructions for use and/or other indication that the compound or composition is suitable or approved for administration to a mammal, e.g., a human, for a CSK mediated disease or condition. The compound or composition may be packaged in unit dose or single dose form, e.g., single dose pills, capsules, or the like.

### VII. Binding Assays

The methods of the present disclosure can involve assays that are able to detect the binding of compounds to a target molecule. Such binding is at a statistically significant level, with a confidence level of at least 90%, or at least 95, 97, 98, 99% or greater confidence level that the assay signal represents binding to the target molecule, i.e., is distinguished from background. In some embodiments, controls are used to distinguish target binding from non-specific binding. A large variety of assays indicative of binding are known for different target types and can be used for this disclosure.

Binding compounds can be characterized by their effect on the activity of the target molecule. Thus, a “low activity” compound has an inhibitory concentration (IC₅₀) or effective concentration (EC₅₀) of greater than 1 μM under standard conditions. By “very low activity” is meant an IC₅₀ or EC₅₀ of above 100 μM under standard conditions. By “extremely low activity” is meant an IC₅₀ or EC₅₀ of above 1 mM under standard conditions. By “moderate activity” is meant an IC₅₀ or EC₅₀ of 200 nM to 1 μM under standard conditions. By “moderately high activity” is meant an IC₅₀ or EC₅₀ of 1 nM to 200 nM. By “high activity” is meant an IC₅₀ or EC₅₀ of below 1 nM under standard conditions. The IC₅₀ or EC₅₀ is defined as the concentration of compound at which 50% of the activity of the target molecule (e.g. enzyme or other protein) activity being measured is lost or gained relative to the range of activity observed when no compound is present. Activity can be measured using methods known to those of ordinary skill in the art, e.g., by measuring any detectable product or signal produced by occurrence of an enzymatic reaction, or other activity by a protein being measured.

By “background signal” in reference to a binding assay is meant the signal that is recorded under standard conditions for the particular assay in the absence of a test compound, molecular scaffold, or ligand that binds to the target molecule. Persons of ordinary skill in the art will realize that accepted methods exist and are widely available for determining background signal.

By “standard deviation” is meant the square root of the variance. The variance is a measure of how spread out a distribution is. It is computed as the average squared deviation of each number from its mean. For example, for the numbers 1, 2, and 3, the mean is 2 and the variance is:

$${o}^{2} = \frac{{( 1 - 2 )}^{2} + {( 2 - 2 )}^{2} + {( 3 - 2 )}^{2} = 0.6 \; 67}{3} .$$

Surface Plasmon Resonance

Binding parameters can be measured using surface plasmon resonance, for example, with a BIAcore® chip (Biacore, Japan) coated with immobilized binding components. Surface plasmon resonance is used to characterize the microscopic association and dissociation constants of reaction between an sFv or other ligand directed against target molecules. Such methods are generally described in the following references which are incorporated herein by reference. Vely F. et al., (2000) BIAcore® analysis to test phosphopeptide-SH2 domain interactions, Methods in Molecular Biology. 121:313-21; Liparoto et al., (1999) Biosensor analysis of the interleukin-2 receptor complex, Journal of Molecular Recognition. 12:316-21; Lipschultz et al., (2000) Experimental design for analysis of complex kinetics using surface plasmon resonance, Methods. 20(3):310-8; Malmqvist., (1999) BIACORE: an affinity biosensor system for characterization of biomolecular interactions, Biochemical Society Transactions 27:335-40; Alfthan, (1998) Surface plasmon resonance biosensors as a tool in antibody engineering, Biosensors & Bioelectronics. 13:653-63; Fivash et al., (1998) BIAcore for macromolecular interaction, Current Opinion in Biotechnology. 9:97-101; Price et al.; (1998) Summary report on the ISOBM TD-4 Workshop: analysis of 56 monoclonal antibodies against the MUC1 mucin. Tumour Biology 19 Suppl 1:1-20; Malmqvist et al, (1997) Biomolecular interaction analysis: affinity biosensor technologies for functional analysis of proteins, Current Opinion in Chemical Biology. 1:378-83; O'Shannessy et al., (1996) Interpretation of deviations from pseudo-first-order kinetic behavior in the characterization of ligand binding by biosensor technology, Analytical Biochemistry. 236:275-83; Malmborg et al., (1995) BIAcore as a tool in antibody engineering, Journal of Immunological Methods. 183:7-13; Van Regenmortel, (1994) Use of biosensors to characterize recombinant proteins, Developments in Biological Standardization. 83:143-51; and O'Shannessy, (1994) Determination of kinetic rate and equilibrium binding constants for macromolecular interactions: a critique of the surface plasmon resonance literature, Current Opinions in Biotechnology. 5:65-71.

BIAcore© uses the optical properties of surface plasmon resonance (SPR) to detect alterations in protein concentration bound to a dextran matrix lying on the surface of a gold/glass sensor chip interface, a dextran biosensor matrix. In brief, proteins are covalently bound to the dextran matrix at a known concentration and a ligand for the protein is injected through the dextran matrix. Near infrared light, directed onto the opposite side of the sensor chip surface is reflected and also induces an evanescent wave in the gold film, which in turn, causes an intensity dip in the reflected light at a particular angle known as the resonance angle. If the refractive index of the sensor chip surface is altered (e.g. by ligand binding to the bound protein) a shift occurs in the resonance angle. This angle shift can be measured and is expressed as resonance units (RUs) such that 1000 RUs is equivalent to a change in surface protein concentration of 1 ng/mm². These changes are displayed with respect to time along the y-axis of a sensorgram, which depicts the association and dissociation of any biological reaction.

High Throughput Screening (HTS) Assays

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell which causes a disease. High throughput methods enable researchers to assay thousands of different chemicals against each target molecule very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra-high-throughput Screening (uHTS) generally refers to the high-throughput screening accelerated to greater than 100,000 tests per day.

To achieve high-throughput screening, it is advantageous to house samples on a multicontainer carrier or platform. A multicontainer carrier facilitates measuring reactions of a plurality of candidate compounds simultaneously. Multi-well microplates may be used as the carrier. Such multi-well microplates, and methods for their use in numerous assays, are both known in the art and commercially available.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity used as a comparator or control. It will be appreciated that modulators can also be combined with the enzyme activators or inhibitors to find modulators which inhibit the enzyme activation or repression that is otherwise caused by the presence of the known the enzyme modulator.

Measuring Enzymatic and Binding Reactions During Screening Assays

Techniques for measuring the progression of enzymatic and binding reactions, e.g., in multicontainer carriers, are known in the art and include, but are not limited to, the following.

Spectrophotometric and spectrofluorometric assays are well known in the art. Examples of such assays include the use of colorimetric assays for the detection of peroxides, as described in Gordon, A. J. and Ford, R. A., (1972) The Chemist's Companion: A Handbook Of Practical Data, Techniques, And References, John Wiley and Sons, N.Y., Page 437.

Fluorescence spectrometry may be used to monitor the generation of reaction products. Fluorescence methodology is generally more sensitive than the absorption methodology. The use of fluorescent probes is well known to those skilled in the art. For reviews, see Bashford et al., (1987) Spectrophotometry and Spectrofluorometry: A Practical Approach, pp. 91-114, IRL Press Ltd.; and Bell, (1981) Spectroscopy In Biochemistry, Vol. I, pp. 155-194, CRC Press.

In spectrofluorometric methods, enzymes are exposed to substrates that change their intrinsic fluorescence when processed by the target enzyme. Typically, the substrate is nonfluorescent and is converted to a fluorophore through one or more reactions. As a non-limiting example, SMase activity can be detected using the Amplex® Red reagent (Molecular Probes, Eugene, Oreg.). In order to measure sphingomyelinase activity using Amplex® Red, the following reactions occur. First, SMase hydrolyzes sphingomyelin to yield ceramide and phosphorylcholine. Second, alkaline phosphatase hydrolyzes phosphorylcholine to yield choline. Third, choline is oxidized by choline oxidase to betaine. Finally, H₂O₂, in the presence of horseradish peroxidase, reacts with Amplex® Red to produce the fluorescent product, Resorufin, and the signal therefrom is detected using spectrofluorometry.

Fluorescence polarization (FP) is based on a decrease in the speed of molecular rotation of a fluorophore that occurs upon binding to a larger molecule, such as a receptor protein, allowing for polarized fluorescent emission by the bound ligand. FP is empirically determined by measuring the vertical and horizontal components of fluorophore emission following excitation with plane polarized light. Polarized emission is increased when the molecular rotation of a fluorophore is reduced. A fluorophore produces a larger polarized signal when it is bound to a larger molecule (i.e. a receptor), slowing molecular rotation of the fluorophore. The magnitude of the polarized signal relates quantitatively to the extent of fluorescent ligand binding. Accordingly, polarization of the “bound” signal depends on maintenance of high affinity binding.

FP is a homogeneous technology and reactions are very rapid, taking seconds to minutes to reach equilibrium. The reagents are stable, and large batches may be prepared, resulting in high reproducibility. Because of these properties, FP has proven to be highly automatable, often performed with a single incubation with a single, premixed, tracer-receptor reagent. For a review, see Owicki et al., (1997), Application of Fluorescence Polarization Assays in High-Throughput Screening, Genetic Engineering News, 17:27.

FP is particularly desirable since its readout is independent of the emission intensity (Checovich, W. J., et al., (1995) Nature 375:254-256; Dandliker, W. B., et al., (1981) Methods in Enzymology 74:3-28) and is thus insensitive to the presence of colored compounds that quench fluorescence emission. FP and FRET (see below) are well-suited for identifying compounds that block interactions between sphingolipid receptors and their ligands. See, for example, Parker et al., (2000) Development of high throughput screening assays using fluorescence polarization: nuclear receptor-ligand-binding and kinase/phosphatase assays, J Biomol Screen 5:77-88.

Fluorophores derived from sphingolipids that may be used in FP assays are commercially available. For example, Molecular Probes (Eugene, Oreg.) currently sells sphingomyelin and one ceramide flurophores. These are, respectively, N-(4,4-difluoro-5,7-dimethyl-4-bora-3a,4a-diaza-s-indacene-3-pentanoyl)sphingosyl phosphocholine (BODIPY® FL C5-sphingomyelin); N-(4,4-difluoro-5,7-dimethyl-4-bora-3a,4a-diaza-s-indacene-3-dodecanoyl)sphingosyl phosphocholine (BODIPY® FL C12-sphingomyelin); and N-(4,4-difluoro-5,7-dimethyl-4-bora-3a,4a-diaza-s-indacene-3-pentanoyl)sphingosine (BODIPY® FL C5-ceramide). U.S. Pat. No. 4,150,949, (Immunoassay for gentamicin), discloses fluorescein-labelled gentamicins, including fluoresceinthiocarbanyl gentamicin. Additional fluorophores may be prepared using methods well known to the skilled artisan.

Exemplary normal-and-polarized fluorescence readers include the POLARION® fluorescence polarization system (Tecan AG, Hombrechtikon, Switzerland). General multiwell plate readers for other assays are available, such as the VERSAMAX® reader and the SPECTRAMAX® multiwell plate spectrophotometer (both from Molecular Devices).

Fluorescence resonance energy transfer (FRET) is another useful assay for detecting interaction and has been described. See, e.g., Heim et al., (1996) Curr. Biol. 6:178-182; Mitra et al., (1996) Gene 173:13-17; and Selvin et al., (1995) Meth. Enzymol. 246:300-345. FRET detects the transfer of energy between two fluorescent substances in close proximity, having known excitation and emission wavelengths. As an example, a protein can be expressed as a fusion protein with green fluorescent protein (GFP). When two fluorescent proteins are in proximity, such as when a protein specifically interacts with a target molecule, the resonance energy can be transferred from one excited molecule to the other. As a result, the emission spectrum of the sample shifts, which can be measured by a fluorometer, such as a fMAX multiwell fluorometer (Molecular Devices, Sunnyvale Calif.).

Scintillation proximity assay (SPA) is a particularly useful assay for detecting an interaction with the target molecule. SPA is widely used in the pharmaceutical industry and has been described (Hanselman et al., (1997) J. Lipid Res. 38:2365-2373; Kahl et al., (1996) Anal. Biochem. 243:282-283; Undenfriend et al., (1987) Anal. Biochem. 161:494-500). See also U.S. Pat. Nos. 4,626,513 and 4,568,649, and European Patent No. 0,154,734. One commercially available system uses FLASHPLATE® scintillant-coated plates (NEN Life Science Products, Boston, Mass.).

The target molecule can be bound to the scintillator plates by a variety of well-known means. Scintillant plates are available that are derivatized to bind to fusion proteins such as GST, His6 or Flag fusion proteins. Where the target molecule is a protein complex or a multimer, one protein or subunit can be attached to the plate first, then the other components of the complex added later under binding conditions, resulting in a bound complex.

In a typical SPA assay, the gene products in the expression pool will have been radiolabeled and added to the wells, and allowed to interact with the solid phase, which is the immobilized target molecule and scintillant coating in the wells. The assay can be measured immediately or allowed to reach equilibrium. Either way, when a radiolabel becomes sufficiently close to the scintillant coating, it produces a signal detectable by a device such as a TOPCOUNT NXT© microplate scintillation counter (Packard BioScience Co., Meriden Conn.). If a radiolabeled expression product binds to the target molecule, the radiolabel remains in proximity to the scintillant long enough to produce a detectable signal.

In contrast, the labeled proteins that do not bind to the target molecule, or bind only briefly, will not remain near the scintillant long enough to produce a signal above background. Any time spent near the scintillant caused by random Brownian motion will also not result in a significant amount of signal. Likewise, residual unincorporated radiolabel used during the expression step may be present, but will not generate significant signal because it will be in solution rather than interacting with the target molecule. These non-binding interactions will therefore cause a certain level of background signal that can be mathematically removed. If too many signals are obtained, salt or other modifiers can be added directly to the assay plates until the desired specificity is obtained (Nichols et al., (1998) Anal. Biochem. 257:112-119).

General Synthesis

The compounds may be prepared using the methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

The compounds of this disclosure may contain one or more asymmetric or chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, supercritical fluid chromathography, chiral seed crystals, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

It will also be appreciated that in each of the schemes, the addition of any substituent may result in the production of a number of isomeric products (including, but not limited to, enantiomers or one or more diastereomers) any or all of which may be isolated and purified using conventional techniques. When enantiomerically pure or enriched compounds are desired, chiral chromatography and/or enantiomerically pure or enriched starting materials may be employed as conventionally used in the art or as described in the Examples.

Compounds of the present disclosure may be synthesized in accordance with the general reaction schemes and/or examples described below. The general schemes may be altered by substitution of the starting materials with other materials having similar structures to result in corresponding products. The structure of the desired product will generally make apparent to a person of skill in the art the required starting materials.

Schemes 1-5 provide exemplary synthetic routes for the synthesis of compounds provided herein (e.g., a compound of Formula I and subembodiments thereof). A compound of Formula I, or other formulas or compounds disclosed herein, is typically prepared by first providing core intermediates and then coupling the core intermediates to form a sulfonamide bond.

Scheme 1 shows an embodiment for synthesis of compounds of Formula I, or other formulas or compounds disclosed herein, comprising the preparation of a core intermediate compound 8.

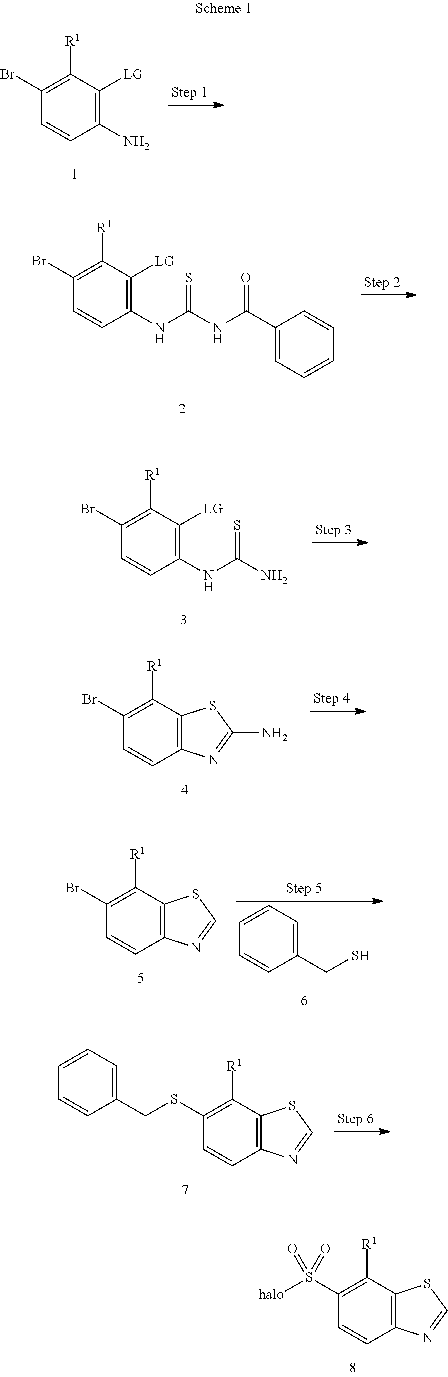

As shown in Scheme 1, starting with a bromo-aniline 1, a reaction with benzoyl isothiocyanate provides a carbamothioylbenzamide compound 2. LG is any suitable leaving group, e.g., halo such as Cl or F. The reaction is conducted in any suitable aprotic solvent at a temperature at about or below about room temperature (e.g., 0° C.). The benzamide in compound 2 is cleaved in the presence of a base (e.g., NaOH or KOH) and an alcohol solvent (e.g., methanol, ethanol, butanol, isoproanol) to provide compound 3. Compound 3 is cyclized in the presence of a strong base such as, e.g., sodium hydride, in an aprotic solvent such as e.g., DMF, to provide compound 4 which is reductively deaminated in the presence of an alkyl nitirite (e.g., tert-butyl nitrite) in aprotic solvents (e.g., dioxane, DMF) to provide compound 5. Compound 5 is coupled with a thiol 6 to provide compound 7 by a suitable transition metal mediated coupling reaction (e.g., a palladium(II) mediated reaction). It will be understood that the bromo group of compound 1 can be replaced with any suitable leaving group that has a reactivity suitable for the thiol coupling reaction of step 5 in Scheme 1. Such leaving groups are known to one of skill in the art and are contemplated within the scope of embodiments presented herein. Non-limiting examples of suitable replacements for the bromo group in compound 1 include chloro or triflate groups. Compound 7 is converted to a sulfonyl halide, compound 8, by reaction with an N-halosuccinimide (e.g., N-chlorosuccinimide, N-bromosuccinimide). R¹ in Scheme 1 is as defined herein in some or any embodiments of Formula I.

Scheme 2 shows a further embodiment for synthesis of compounds of Formula I, or other formulas or compounds disclosed herein, comprising the preparation of a core intermediate compound 13.

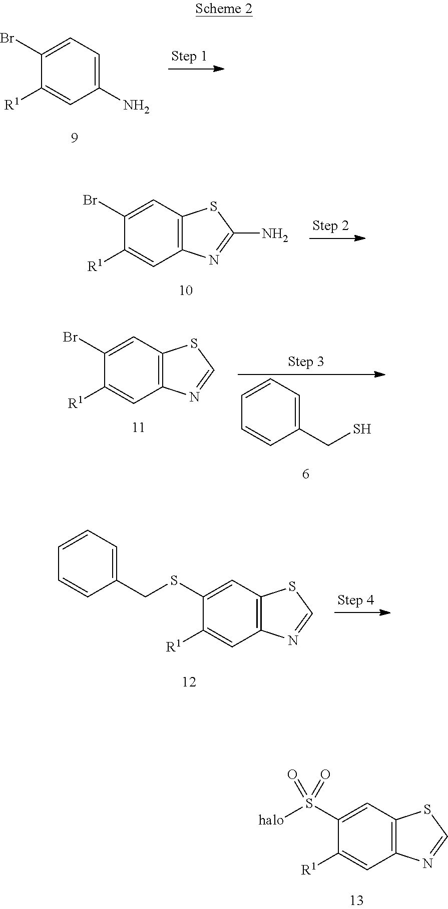

As shown in Scheme 2, starting with a bromo-aniline 9, a reaction with potassium thiocyanate in the presence of acetic acid provides a cyclized compound 10 which is subjected to reductive deamination in the presence of an alkyl nitirite (e.g., tert-butyl nitrite) in aprotic solvents (e.g., dioxane, DMF) to provide compound 11. Compound 11 is coupled with a thiol 6 to provide compound 12 by a suitable transition metal mediated coupling reaction (e.g., a palladium(II) mediated reaction). It will understood that the bromo group of compound 9 can be replaced with any suitable leaving group that has a reactivity suitable for the thiol coupling reaction of step 3 in Scheme 2. Such leaving groups are known to one of skill in the art and are contemplated within the scope of embodiments presented herein. Non-limiting examples of suitable replacements for the bromo group in compound 9 include chloro or triflate groups. Compound 12 is converted to a sulfonyl halide, compound 13, by reaction with an N-halosuccinimide (e.g., N-chlorosuccinimide, N-bromosuccinimide). R¹ in Scheme 2 is as defined herein in some or any embodiments of Formula I.

Scheme 3 shows an embodiment for synthesis of compounds of Formula I, or other formulas or compounds disclosed herein, comprising the preparation of a core intermediate compound 19A and a core intermediate compound 20A.

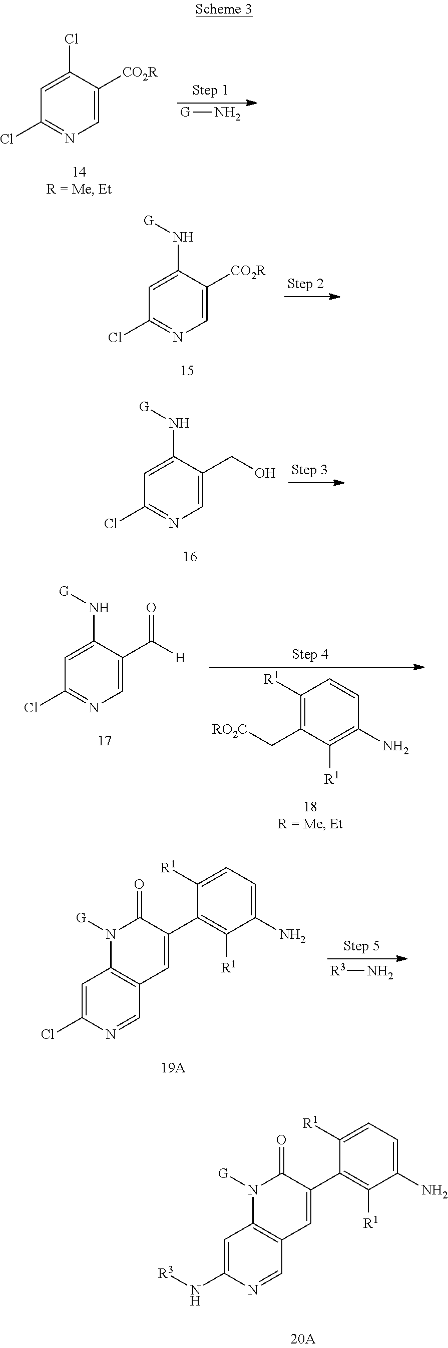

Starting with compound 14, a displacement reaction with amine G-NH₂ provides compound 15. The displacement reaction with amine G-NH₂ is conducted in the presence of an aprotic solvent (e.g., acetonitrile, dioxane), an organic amine (e.g., triethylamine, diisopropylamine, N,N-diisopropylethylamine) and by heating the reaction mixture at a temperature ranging from about 20° C. to about 150° C., from about 20° C. to about 100° C., or from about 20° C. to about 50° C. for a period of time ranging from about 12 hours to about 72 hours or till completion of the reaction. The ester group in compound 15 is reduced to an alcohol in the presence of a suitable reducing agent (e.g., lithium aluminum hydrided) to obtain compound 16. Compound 16 is oxidized to an aldehyde 17 in the presence of a suitable oxidant (e.g., manganese dioxide). A reaction of compound 17 with compound 18 yields a core intermediate compound 19A.

In one embodiment, a reaction of compound 19A with an amine R³—NH₂ provides intermediate compound 20A. The reaction with the amine R³—NH₂ is conducted in solvents such as pyridine at temperatures ranging from 0° C. to about room temperature, or in the presence of an organic base (e.g., triethylamine, N,N-diisopropylethylamine) in aprotic solvents such as dioxane and by heating the reaction mixture at temperatures ranging from room temperature to about 160° C. In further embodiments, the amine may react with the heteroaryl chloro group in compound 9A via a transition metal mediated coupling reaction (e.g., palladium(II) mediated coupling). Other methods for reacting an amine with the heteroaryl chloro group of compound 19A will be apparent to one of skill in the art and are contemplated within the scope of embodiments presented herein. G in Scheme 3 is as defined herein in some or any embodiments of Formula I.

In another embodiment, a reaction of compound 19A with para-methoxybenzylamine or tert-butyl carbamate provides intermediate compound 20B as shown in Scheme 4.

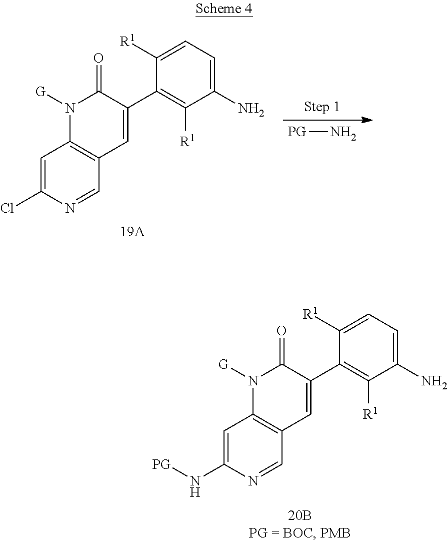

In Scheme 4, the reaction of the amine with the heteroaryl chloro group of compound 19A is conducted in solvents such as pyridine at temperatures ranging from 0° C. to about room temperature, or in the presence of an organic base (e.g., triethylamine, N,N-diisopropylethylamine) in aprotic solvents such as dioxane and by heating the reaction mixture at temperatures ranging from room temperature to about 160° C. In further embodiments, the amine may react with the heteroaryl chloro group in compound 19A via a transition metal mediated coupling reaction (e.g., palladium(II) mediated coupling). Other methods for reacting an amine with the heteroaryl chloro group of compound 19A will be apparent to one of skill in the art and are contemplated within the scope of embodiments presented herein.

Scheme 5 shows the coupling of intermediate compounds 8 or 13 with intermediate compounds 20A or 20B to provide compounds of Formula I or other formulas or compounds disclosed herein.

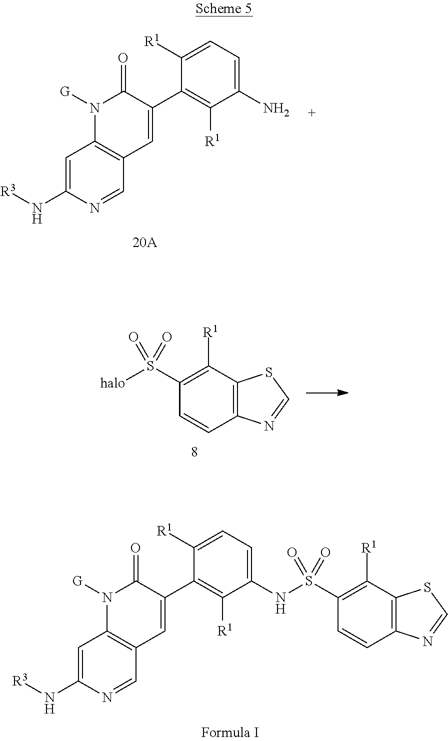

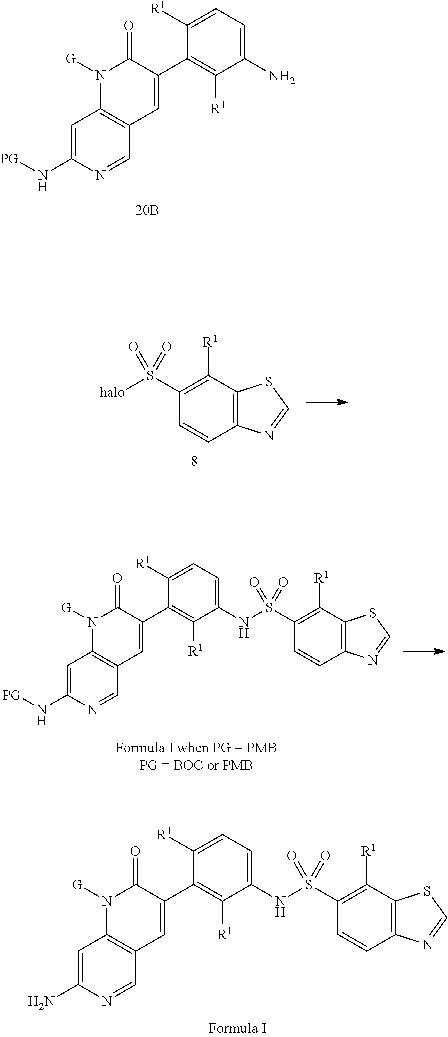

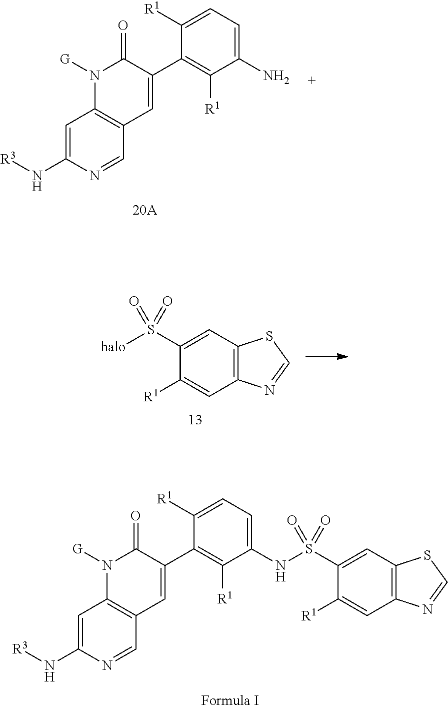

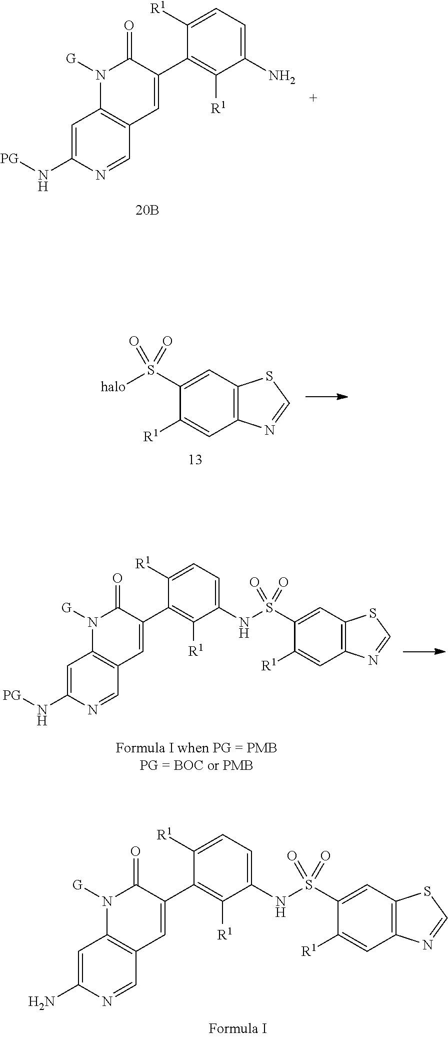

The reaction of compound 20A with intermediate 8 or 13 provides compounds of Formula I. Similarly the reaction of compound 20B with intermediate 8 or 13 provides, in some embodiments, compounds of Formula I. In additional embodiments, removal of the protecting group PG provides further compounds of Formula I. The reaction of the amine 20A or 20B with the sulfonyl halide 8 or 13 is conducted in the presence of pyridine at temperatures ranging from about 0° C. to about room temperature. The BOC or PMB protecting groups are removed under standard conditions for deprotection (e.g., in the presence of trifluoroacetic acid).

### Intermediate 8

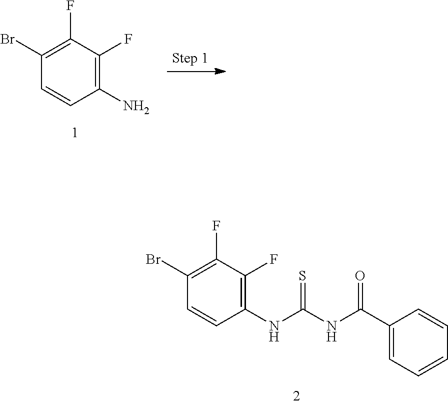 [SMILES: Nc1ccc(Br)c(F)c1F ; O=C(NC(=S)Nc1ccc(Br)c(F)c1F)c1ccccc1]

Step 1. Preparation of N-[(4-bromo-2,3-difluoro-phenyl)carbamothioyl]benzamide 2: To a round bottom flask containing a stir bar was added 4-bromo-2,3-difluoro-aniline (1, 35.0 g, 168 mmol) and acetone (300 mL). The reaction was placed under N₂ and cooled to 0° C., and benzoyl isothiocyanate (35.0 mL, 260 mmol) was added slowly, dropwise, by syringe. The reaction was stirred at 0° C. for 1 h, then warmed slowly to 20° C. over 17 h, giving a white precipitate. The white precipitate was collected by vacuum filtration and dried, giving the desired product (2, 49 g, 79% yield).

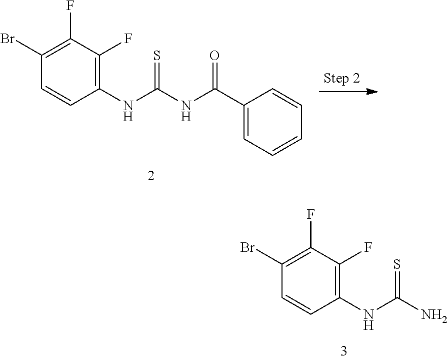 [SMILES: O=C(NC(=S)Nc1ccc(Br)c(F)c1F)c1ccccc1 ; NC(=S)Nc1ccc(Br)c(F)c1F]

Step 2. Preparation of (4-bromo-2,3-difluoro-phenyl)thiourea 3: To each of two round bottom flasks containing a stir bar was added N-[(4-bromo-2,3-difluoro-phenyl)carbamothioyl]benzamide (2, 24.5 g, 66.0 mmol), MeOH (25 mL), and NaOH (2.0 M, 172 mL, 344 mmol). The reactions were placed under N₂ and heated to 70° C. for 2 h. LC/ESI-MS analysis gave a large peak for the desired product with no remaining starting material. The reactions were combined and extracted with ethyl acetate (3×250 mL). The organic fraction was washed with 5 M NaCl (1×250 mL), dried over anhydrous Na₂SO₄, filtered, and evaporated, giving the desired product (3, 32 g, 90% yield).

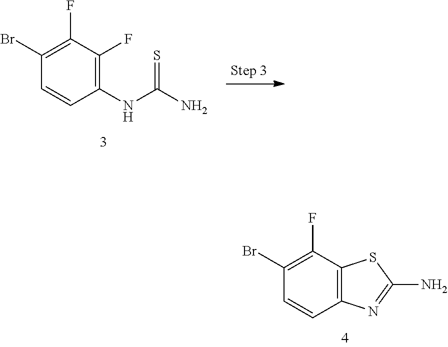 [SMILES: NC(=S)Nc1ccc(Br)c(F)c1F ; Nc1nc2ccc(Br)c(F)c2s1]

Step 3. Preparation of 6-bromo-7-fluoro-1,3-benzothiazol-2-amine 4: To a round bottom flask containing a stir bar was added (4-bromo-2,3-difluoro-phenyl)thiourea (3, 8.0 g, 29.9 mmol) and DMF (80 mL). The solution was placed under N₂ and stirred at 20° C., and sodium hydride (60 wt pct dispersion in mineral oil, 2.78 g, 69.5 mmol) was added slowly, in small portions, over 5 m. The reaction was stirred at 20° C. for 15 m, then heated to 70° C. for 1 h. LC/ESI-MS analysis gave a large peak for the desired product with a large peak for remaining starting material. The reaction was allowed to stir at 70° C. for an additional 30 m. LC/ESI-MS analysis gave a large peak for the desired product with a large peak for remaining starting material, unimproved form initial analyses at 1 h. The reaction was cooled to 20° C. and additional sodium hydride (60 wt pct dispersion in mineral oil, 1.8 g, 45.0 mmol) was added slowly, in small portions, over 5 m. The reaction was stirred at 20° C. for 15 m, then heated to 70° C. for 1 h. LC/ESI-MS analysis gave a large peak for the desired product with a small peak for remaining starting material. The reaction was cooled to 20° C. and added to water (300 mL), giving an off white precipitate. The off white precipitate was collected by vacuum filtration, washed with water (3×100 mL), and dried, giving the desired product. The procedure detailed above was performed over 4 replicates to produce 4 equivalent batches of material, giving the desired product (4, 26.2 g, 89% yield). MS (ESI) [M+H⁺]⁺=246.9, 248.9.

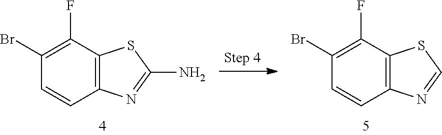 [SMILES: Nc1nc2ccc(Br)c(F)c2s1 ; Fc1c(Br)ccc2ncsc12]

Step 4. Preparation of 6-bromo-7-fluoro-1,3-benzothiazole 5: To each of two round bottom flasks containing a stir bar was added 6-bromo-7-fluoro-1,3-benzothiazol-2-amine (4, 13.1 g, 53.0 mmol) and 1,4-dioxane (250 mL). The reactions were stirred at 20° C., and tert-butyl nitrite (21.0 mL, 159 mmol) was added slowly, dropwise, by syringe. The reactions were allowed to stir for 15 m, then heated to 90° C. for 1 h. LC/ESI-MS analysis gave a large peak for the desired product with a small peak for remaining starting material. The reactions were combined, evaporated, added to 5.3 M NH₄Cl (500 mL) and extracted with EtOAc (2×500 mL). The organic fraction was washed with water (1×500 mL) and 5 M NaCl (1×500 mL), dried over anhydrous Na₂SO₄, filtered, evaporated, and purified by normal phase flash column chromatography (SiO₂, 0-100% EtOAc in hexanes), giving the desired product (5, 6.79 g, 28% yield).

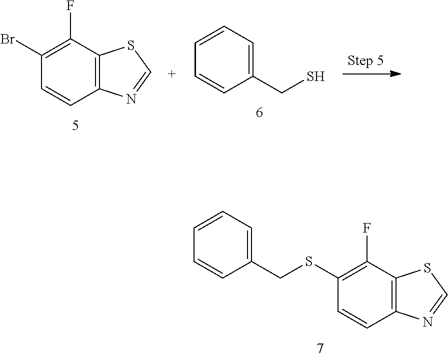

Step 5. Preparation of 6-benzylsulfanyl-7-fluoro-1,3-benzothiazole 7: To a pressure vessel containing a stir bar was added 6-bromo-7-fluoro-1,3-benzothiazole (5, 6.79 g, 29.3 mmol), phenylmethanethiol (6, 3.8 mL, 4.02 g, 32.4 mmol), 4,5-bis(diphenylphosphino)-9,9-dimethylxanthene (XantPhos, 1.59 g, 2.75 mmol), palladium(II) acetate (0.33 g, 1.45 mmol), N,N-diisopropylethylamine (12.0 mL, 8.90 g, 68.9 mmol), and 1,4-dioxane (75.0 mL). The reaction vessel was placed under N₂, sealed, and heated to 100° C. for 17 h. LC/ESI-MS analysis gave a large peak for the desired product with no remaining starting material. The reaction was added to 5.3 M NH₄Cl (500 mL) and extracted with EtOAc (2×250 mL). The organic fraction was washed with water (1×500 mL) and 5 M NaCl (1×500 mL), dried over anhydrous Na₂SO₄, filtered, evaporated, and purified by normal phase flash column chromatography (SiO₂, 0-25% EtOAc in hexanes), giving the desired product (7, 7.61 g, 95% yield). MS (ESI) [M+H⁺]⁺=275.9.

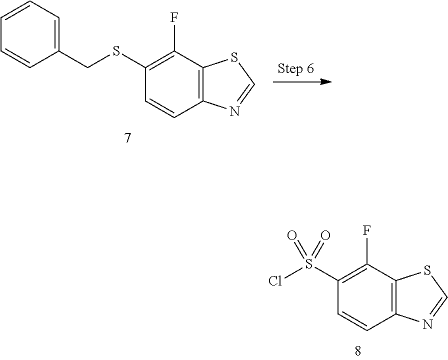 [SMILES: Fc1c(SCc2ccccc2)ccc2ncsc12 ; O=S(=O)(Cl)c1ccc2ncsc2c1F]

Step 6. Preparation of 7-fluoro-1,3-benzothiazole-6-sulfonyl chloride 8: To a round bottom flask containing a stir bar was added 6-benzylsulfanyl-7-fluoro-1,3-benzothiazole (7, 3914 mg, 14.2 mmol), water (5.0 mL, 5000 mg, 277 mmol), and AcOH (45.0 mL). The reaction was stirred vigorously at 20° C., and N-chlorosuccinimide (5699 mg, 42.7 mmol) was added slowly, in small portions, over 1 m. The reaction was stirred vigorously at 20° C. for 1 h. LC/ESI-MS analysis gave a large peak for the desired product with no remaining starting material. The reaction was evaporated, giving product (8, 9830 mg, assumed 99.8% yield, 36.3% purity based on wt) which was used in subsequent reactions as is without further purification. MS (ESI) [M+H⁺]⁺=251.9.

Intermediate 13

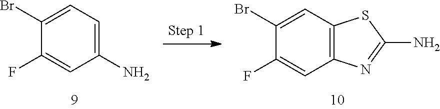 [SMILES: Nc1ccc(Br)c(F)c1 ; Nc1nc2cc(F)c(Br)cc2s1]

Step 1. Preparation of 6-bromo-5-fluoro-1,3-benzothiazol-2-amine 10. To a round bottom flask containing a stir bar was added 4-bromo-3-fluoro-aniline (9, 4762 mg, 25.1 mmol), potassium thiocyanate (9680 mg, 99.61 mmol) and AcOH (90.0 mL). The reaction was stirred vigorously at 20° C., then bromine (1288 uL, 4005 mg, 25.1 mmol in AcOH (3.0 mL)) was added slowly, dropwise, by syringe. The reaction was stirred at 20° C. for 17 h. LC/ESI-MS analysis gave a large peak for the desired product with no remaining starting material. The reaction was evaporated, added to water (500 mL), and adjusted to pH 6 with 1.2 M NaHCO₃, giving a pale yellow precipitate. The precipitate was sonicated extensively and then collected by vacuum filtration, giving the product (10, 5326 mg, 74% yield). MS (ESI) [M+H⁺]⁺=246.9, 248.9.

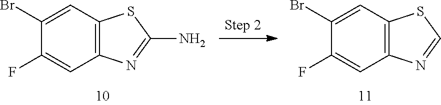 [SMILES: Nc1nc2cc(F)c(Br)cc2s1 ; Fc1cc2ncsc2cc1Br]

Step 2. Preparation of 6-bromo-5-fluoro-1,3-benzothiazole 11. To a dried 20 mL glass vial containing a stir bar was added 6-bromo-5-fluoro-1,3-benzothiazol-2-amine (10, 2471 mg, 10 mmol) and 1,4-dioxane (15 mL). The reaction was stirred at 20° C. and then tert-butyl nitrite (1800 uL, 1560 mg, 15.1 mmol) was added slowly, dropwise, by syringe. The reaction was stirred vigorously at 20° C. for 1 h, then heated to 90° C. for 2 h, giving a dark reddish orange solution. LC/ESI-MS analysis gave a large peak for the desired product with no remaining starting material. The reaction was added to 5.3 M NH₄Cl (300 mL) and extracted with EtOAc (2×300 mL). The organic fraction was washed with water (1×300 mL) and 5 M NaCl (1×300 mL), dried over anhydrous Na₂SO₄, filtered, evaporated and purified by normal phase flash column chromatography (SiO₂, 0-25% EtOAc in hexanes), giving the desired product (11, 1116 mg, 47% yield). MS (ESI) [M+H⁺]⁺=231.9.

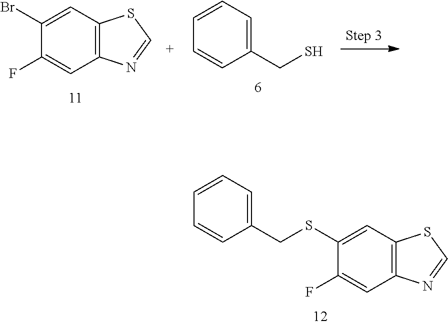

Step 3. Preparation of 6-benzylsulfanyl-5-fluoro-1,3-benzothiazole 12. To a dried 20 mL microwavable vial containing a stir bar was added 6-bromo-5-fluoro-1,3-benzothiazole (11, 1241 mg, 5.35 mmol), phenylmethanethiol (6, 692 uL, 732 mg, 5.90 mmol), 4,5-bis(diphenylphosphino)-9,9-dimethylxanthene (XantPhos, 311 mg, 0.537 mmol), palladium(II) acetate (63.2 mg, 0.282 mmol), N,N-diisopropylethylamine (2.2 mL, 1632 mg, 12.6 mmol), and 1,4-dioxane (10.0 mL). The reaction vial was placed under N₂, sealed, and heated to 100° C. for 17 h. LC/ESI-MS analysis gave a large peak for the desired product with no remaining starting material. The reaction was added to 5.3 M NH₄Cl (300 mL) and extracted with EtOAc (2×300 mL). The organic fraction was washed with water (1×100 mL) and 5 M NaCl (1×100 mL), dried over anhydrous Na₂SO₄, filtered, evaporated and purified by normal phase flash column chromatography (SiO₂, 0-50% EtOAc in hexanes), giving the desired product (12, 1421 mg, 97% yield). MS (ESI) [M+H⁺]⁺=275.9.

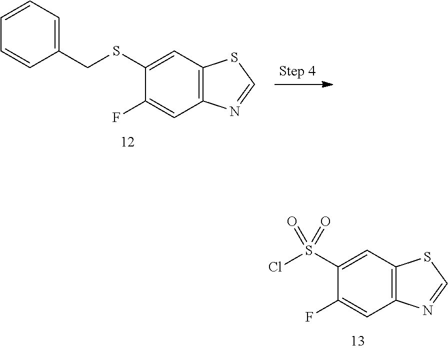 [SMILES: Fc1cc2ncsc2cc1SCc1ccccc1 ; O=S(=O)(Cl)c1cc2scnc2cc1F]

Step 4. Preparation of 5-fluoro-1,3-benzothiazole-6-sulfonyl chloride 13. To a vial containing a stir bar was added 6-benzylsulfanyl-5-fluoro-1,3-benzothiazole (12, 1421 mg, 5.16 mmol), water (1.0 mL, 1000 mg, 55.5 mmol), and AcOH (10 mL). The reaction was stirred vigorously at 20° C., and N-chlorosuccinimide (2069 mg, 15.5 mmol) was added slowly, in small portions, over 1 m. The reaction was stirred vigorously at 20° C. for 1 h. LC/ESI-MS analysis gave a large peak for the desired product with no remaining starting material. To the reaction was added to water (20 mL), giving a pale yellow precipitate. The precipitate was collected by vacuum filtration, washed with water (1×10 mL), and dried, giving the desired product (13, 1232 mg, 93% yield). MS (ESI) [M+H⁺]⁺=251.9.

### Example 1

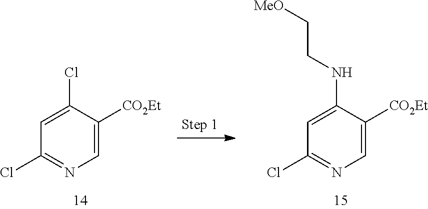 [SMILES: CCOC(=O)c1cnc(Cl)cc1Cl ; CCOC(=O)c1cnc(Cl)cc1NCCOC]

Step 1. Preparation of ethyl 6-chloro-4-(2-methoxyethylamino)pyridine-3-carboxylate 15: To a pressure vessel containing a stir bar was added ethyl 4,6-dichloropyridine-3-carboxylate (14, 2.20 g, 10.0 mmol), 2-methoxyethanamine (901 mg, 12.0 mmol), N,N-diisopropylethylamine (8.0 mL, 5936 mg, 45.93 mmol), and 1,4-dioxane (16.0 mL). The reaction was placed under N₂, sealed, and heated to 100° C. for 17 h. LC/ESI-MS analysis gave a large peak for the desired product with no remaining starting material. The reaction was evaporated, added to 5.3 M NH₄Cl (100 mL), and extracted with EtOAc (2×100 mL). The organic fraction was washed with water (1×100 mL) and 5 M NaCl (1×100 mL), dried over anhydrous Na₂SO₄, filtered, and evaporated, giving the desired product as a white solid (15, 2587 mg, 100% yield). MS (ESI) [M+H⁺]⁺=259.0.

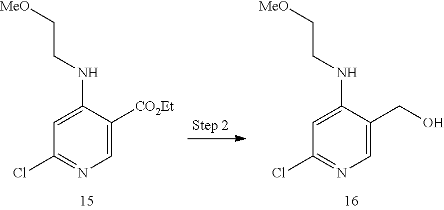 [SMILES: CCOC(=O)c1cnc(Cl)cc1NCCOC ; COCCNc1cc(Cl)ncc1CO]

Step 2. Preparation of [6-chloro-4-(2-methoxyethylamino)-3-pyridyl]methanol 16: To a flask containing a stir bar was added ethyl 6-chloro-4-(2-methoxyethylamino)pyridine-3-carboxylate (15, 2587 mg, 10.0 mmol) and THF (50 mL). The reaction was placed under N₂ and cooled to 0° C., and lithium aluminum hydride (2.0 M in THF, 5.5 mL, 11.0 mmol) was added slowly, dropwise, by syringe. Vigorous bubbling was immediately observed upon addition. The reaction was stirred at 0° C. for 1 h, then warmed slowly to 20° C. over 1 h. LC/ESI-MS analysis gave a large peak for the desired product with no remaining starting material. The reaction was quenched with water (417 uL, 417 mg, 23.1 mmol), NaOH (4.0 M, 276 uL, 1.10 mmol), and water (1300 uL, 1300 mg, 72.1 mmol). The reaction was diluted with CH₂Cl₂ (100 mL), dried over MgSO₄, filtered, and evaporated, giving the desired product (16, 2167 mg, 97% yield).

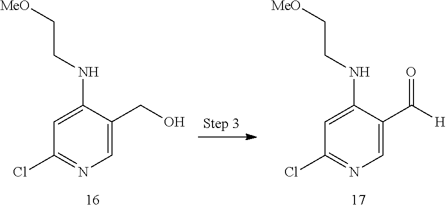 [SMILES: COCCNc1cc(Cl)ncc1CO ; COCCNc1cc(Cl)ncc1C=O]

Step 3. Preparation of 6-chloro-4-(2-methoxyethylamino)pyridine-3-carbaldehyde 17: To a round bottom flask containing a stir bar was added [6-chloro-4-(2-methoxyethylamino)-3-pyridyl]methanol (16, 2167 mg, 10.0 mmol), manganese dioxide (1739 mg, 20.0 mmol) and EtOAc (100 mL). The reaction was heated to 80° C. for 2 h. LC/ESI-MS analysis showed a large peak for the desired product with remaining starting material. To the reaction was added additional manganese dioxide (1739 mg, 20.0 mmol). The reaction was heated to 80° C. for an additional 4 h. LC/ESI-MS analysis gave a large peak for the desired product with no remaining starting material. The reaction was filtered through celite, evaporated, and purified by normal phase flash column chromatography (SiO₂, 0-50% EtOAc in hexanes), giving the desired product (17, 1500 mg, 70% yield). MS (ESI) [M+H⁺]⁺=215.1.

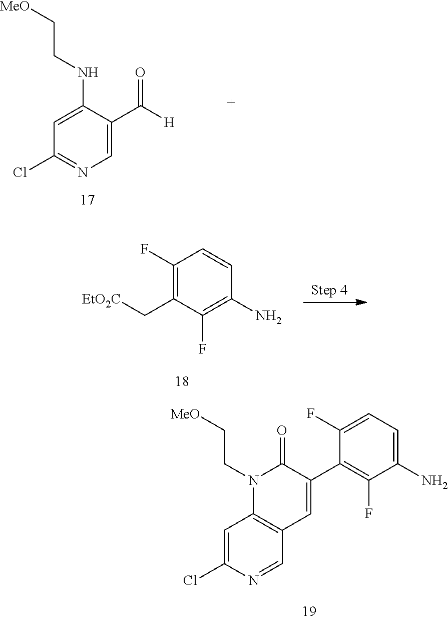 [SMILES: COCCNc1cc(Cl)ncc1C=O ; CCOC(=O)Cc1c(F)ccc(N)c1F ; COCCn1c(=O)c(-c2c(F)ccc(N)c2F)cc2cnc(Cl)cc21]

Step 4. Preparation of 3-(3-amino-2,6-difluoro-phenyl)-7-chloro-1-(2-methoxyethyl)-1,6-naphthyridin-2-one 19: To a pressure vessel containing a stir bar was added 6-chloro-4-(2-methoxyethylamino)pyridine-3-carbaldehyde (17, 1500 mg, 6.99 mmol), ethyl 2-(3-amino-2,6-difluoro-phenyl)acetate (18, 2256 mg, 10.5 mmol), potassium carbonate (2898 mg, 21 mmol), and DMF (30.0 mL). The reaction was placed under N₂, sealed, and heated to 120° C. for 17 h. LC/ESI-MS analysis gave a large peak for the desired, cyclized product with no remaining starting material. The reaction was added to water (300 mL), and extracted with EtOAc (2×300 mL). The organic fraction was washed with water (1×100 mL), and 5 M NaCl (1×100 mL), dried over anhydrous Na₂SO₄, filtered, evaporated, and purified by normal phase flash column chromatography (SiO₂, 0-50% EtOAc in hexanes), giving the desired product (19, 1320 mg, 52% yield). MS (ESI) [M+H⁺]⁺=366.0.

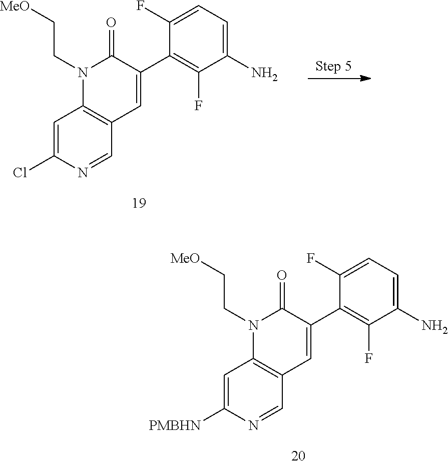

Step 5. Preparation of 3-(3-amino-2,6-difluorophenyl)-7-((4-methoxybenzyl)amino)-1-(2-methoxyethyl)-1,6-naphthyridin-2(1H)-one 20. To a vial containing a stir bar was added 3-(3-amino-2,6-difluoro-phenyl)-7-chloro-1-(2-methoxyethyl)-1,6-naphthyridin-2-one (19, 1320 mg, 3.61 mmol), (4-methoxyphenyl)methanamine (1886 uL, 1980 mg, 14.4 mmol), N,N-diisopropylethylamine (3.33 mL, 2471 mg, 19.1 mmol), and 1,4-dioxane (6.66 mL). The reaction vial was placed under N₂, sealed, and heated to 155° C. for 3 d. LC/ESI-MS analysis gave a large peak for the desired product with a very small peak for remaining starting material. The reaction was evaporated and precipitated from EtOAc (5 mL), giving the desired product (20, 1330 mg, 71% yield). MS (ESI) [M+H⁺]⁺=467.1.

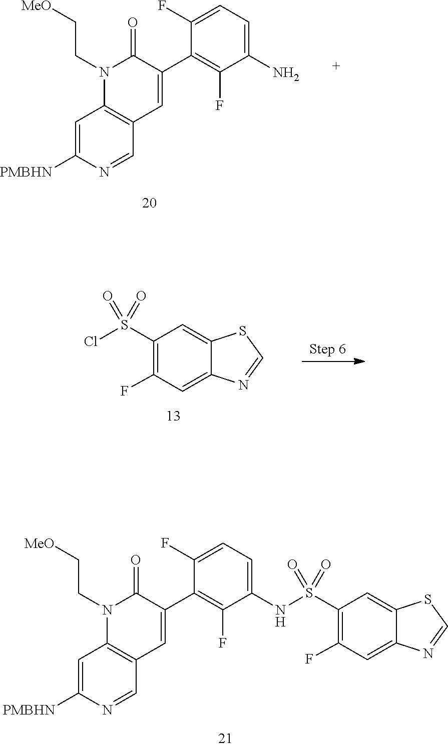

Step 6. Preparation of N-(2,4-difluoro-3-(7-((4-methoxybenzyl)amino)-1-(2-methoxyethyl)-2-oxo-1,2-dihydro-1,6-naphthyridin-3-yl)phenyl)-5-fluorobenzo[d]thiazole-6-sulfonamide 21. To a vial containing a stir bar was added 3-(3-amino-2,6-difluorophenyl)-7-((4-methoxybenzyl)amino)-1-(2-methoxyethyl)-1,6-naphthyridin-2(1H)-one (20, 103 mg, 0.199 mmol) and pyridine (1.0 mL, 944 mg, 11.9 mmol). The reaction was placed under N₂ and cooled to 0° C., and 5-fluoro-1,3-benzothiazole-6-sulfonyl chloride (13, 75 mg, 0.298 mmol) was added slowly, in small portions, over 1 m. The reaction was stirred at 0° C. for 1 h, then warmed slowly to 20° C. over 3 h. LC/ESI-MS analysis gave a large peak for the desired product with no remaining starting material. The reaction was evaporated, dissolved in EtOAc (100 mL), washed with 0.5 M HCl (1×100 mL), water (1×100 mL), and 5 M NaCl (1×100 mL), dried over anhydrous Na₂SO₄, filtered, evaporated, and purified by normal phase flash column chromatography (SiO₂, 0-50% EtOAc in hexanes), giving the desired product (21, 74.8 mg, 55.2% yield). MS (ESI) [M+H⁺]⁺=682.0.

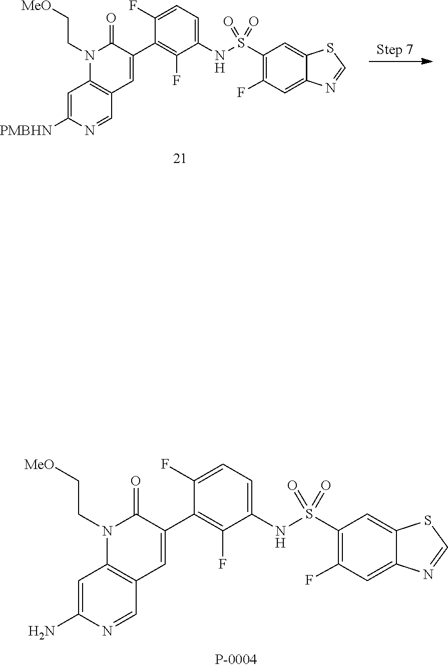

Step 7. Preparation of N-(3-(7-amino-1-(2-methoxyethyl)-2-oxo-1,2-dihydro-1,6-naphthyridin-3-yl)-2,4-difluorophenyl)-5-fluorobenzo[d]thiazole-6-sulfonamide (P-0004). To a vial containing a stir bar was added N-(2,4-difluoro-3-(7-((4-methoxybenzyl)amino)-1-(2-methoxyethyl)-2-oxo-1,2-dihydro-1,6-naphthyridin-3-yl)phenyl)-5-fluorobenzo[d]thiazole-6-sulfonamide (21, 75 mg, 0.110 mmol), trifluoroacetic acid (500 uL, 745 mg, 6.53 mmol), and CH₂Cl₂ (1.5 mL). The reaction was placed under N₂, sealed, and heated to 65° C. for 4 h. LC/ESI-MS analysis gave a large peak for the desired product with no remaining starting material. The reaction was evaporated and purified by reverse phase flash column chromatography (C18, 0-100% CH₃CN (0.1% HCO2H), water (0.1% HCO₂H)), giving the desired product (P-0004, 45 mg, 71% yield). MS (ESI) [M+H⁺]=561.9.

### Example 2

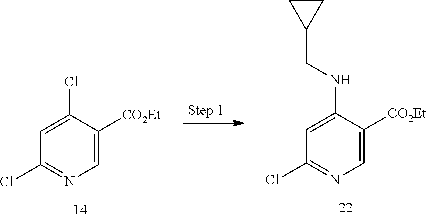 [SMILES: CCOC(=O)c1cnc(Cl)cc1Cl ; CCOC(=O)c1cnc(Cl)cc1NCC1CC1]

Step 1. Preparation of ethyl 6-chloro-4-(cyclopropylmethylamino)pyridine-3-carboxylate 22. To a round bottom flask containing a stir bar was added ethyl 4,6-dichloropyridine-3-carboxylate (14, 11009 mg, 50.0 mmol) and CH₃CN (50.0 mL). The reaction was stirred at 20° C. and triethylamine (8.0 mL, 5808 mg, 57.4 mmol) was added slowly, dropwise, by syringe. To the reaction was added, slowly, dropwise, by syringe, cyclopropylmethylamine (4.8 mL, 3936 mg, 55.3 mmol). The reaction was stirred at 20° C. for 1 m, then heated to 50° C. for 17 h. LC/ESI-MS analysis gave a large peak for the desired product with a small peak for remaining starting material. The reaction was added to 5.3 M NH₄Cl (250 mL) and extracted with EtOAc (2×250 mL). The organic fraction was washed with water (1×100 mL) and 5.0 M NaCl (1×100 mL), dried over anhydrous Na₂SO₄, filtered, and evaporated, giving the desired product (22, 12.3 g, 97% yield). MS (ESI) [M+H⁺]⁺=255.0.

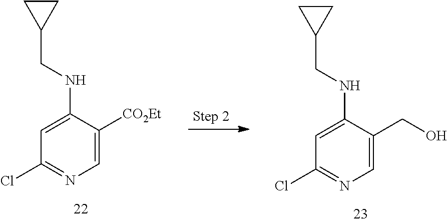 [SMILES: CCOC(=O)c1cnc(Cl)cc1NCC1CC1 ; OCc1cnc(Cl)cc1NCC1CC1]

Step 2. Preparation of [6-chloro-4-(cyclopropylmethylamino)-3-pyridyl]methanol 23. To a round bottom flask containing a stir bar was added lithium aluminum hydride (1.0 M in THF, 100 mL, 3.80 g, 100 mmol). The reaction was placed under N₂ and cooled to 0° C., and ethyl 6-chloro-4-(cyclopropylmethylamino)pyridine-3-carboxylate (22, 12.4 g, 48.5 mmol, in THF (100.0 mL)) was added slowly, dropwise, by addition funnel. Vigorous bubbling was immediately observed upon addition. The reaction was stirred at 0° C. for 1 h, then warmed slowly to 20° C. over 17 h, giving a slightly cloudy pale orange solution. LC/ESI-MS analysis gave a large peak for the desired product with no remaining starting material. The reaction was carefully added to a vigorously stirring solution of Na₂SO₄ decahydrate (158.7 g, 492.5 mmol, in THF (200 mL), 0° C.). The reaction was stirred at 0° C. for 5 m, then warmed to 20° C. over 1 h. The reaction was filtered, dried over anhydrous Na₂SO₄, filtered, evaporated, and purified by normal phase flash column chromatography (SiO₂, 0-10% MeOH in CH₂Cl₂), giving the desired product (23, 8517 mg, 83% yield).

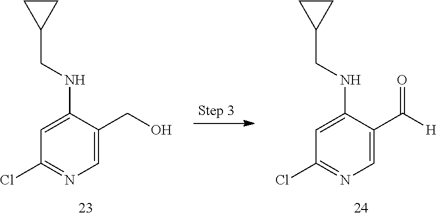 [SMILES: OCc1cnc(Cl)cc1NCC1CC1 ; O=Cc1cnc(Cl)cc1NCC1CC1]

Step 3. Preparation of 6-chloro-4-(cyclopropylmethylamino)pyridine-3-carbaldehyde 24. To a round bottom flask containing a stir bar was added [6-chloro-4-(cyclopropylmethylamino)-3-pyridyl]methanol (23, 8517 mg, 40.0 mmol) and CH₂Cl₂ (400 mL, minimum required for solubility). The reaction was stirred vigorously at 20° C., and manganese dioxide (activated, 10470 mg, 120.4 mmol) was added slowly, in small portions, over 5 m. The reaction was stirred vigorously at 20° C. for 3 d, giving an opaque black solution. LC/ESI-MS analysis gave a large peak for the desired product with no remaining starting material. The reaction was filtered through celite and evaporated, giving the desired product (24, 8355 mg, 97% yield). MS (ESI) [M+H⁺]⁺=211.1.

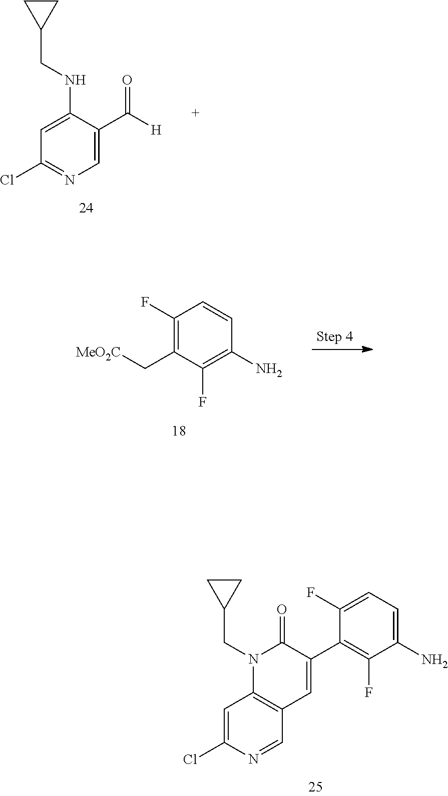 [SMILES: O=Cc1cnc(Cl)cc1NCC1CC1 ; COC(=O)Cc1c(F)ccc(N)c1F ; Nc1ccc(F)c(-c2cc3cnc(Cl)cc3n(CC3CC3)c2=O)c1F]

Step 4. Preparation of 3-(3-amino-2,6-difluoro-phenyl)-7-chloro-1-(cyclopropylmethyl)-1,6-naphthyridin-2-one 25. To a round bottom flask containing a stir bar was added 6-chloro-4-(cyclopropylmethylamino)pyridine-3-carbaldehyde (24, 4.48 g, 21.3 mmol), methyl 2-(3-amino-2,6-difluoro-phenyl)acetate (18, 12.8 g, 63.8 mmol), potassium carbonate (17.6 g, 127.6 mmol), and NMP (200 mL). The reaction was placed under N₂ and heated to 120° C. for 3 d. LC/ESI-MS analysis gave a large peak for the desired product with no remaining starting material. The reaction was evaporated, added to water (750 mL), and extracted with EtOAc (3×250 mL). The organic fraction was washed with 5 M NaCl (6×250 mL), dried over anhydrous Na₂SO₄, filtered, evaporated and purified by normal phase flash column chromatography (SiO₂, 0-100% EtOAc in hexanes), giving the desired product (25, 2241 mg, 28% yield). MS (ESI) [M+H⁺]⁺=362.0.

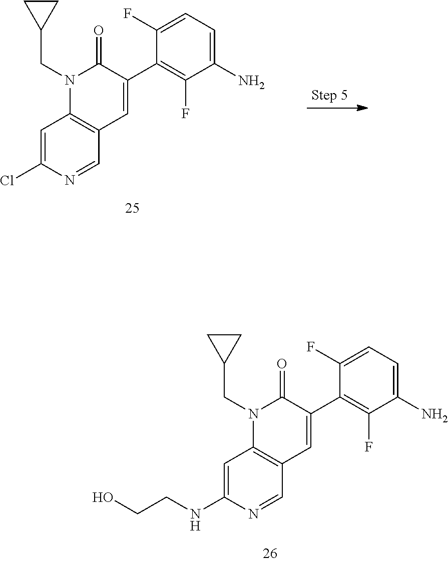 [SMILES: Nc1ccc(F)c(-c2cc3cnc(Cl)cc3n(CC3CC3)c2=O)c1F ; Nc1ccc(F)c(-c2cc3cnc(NCCO)cc3n(CC3CC3)c2=O)c1F]

Step 5. Preparation of 3-(3-amino-2,6-difluoro-phenyl)-1-(cyclopropylmethyl)-7-(2-hydroxyethylamino)-1,6-naphthyridin-2-one 26. To a vial containing a stir bar was added 3-(3-amino-2,6-difluoro-phenyl)-7-chloro-1-(cyclopropylmethyl)-1,6-naphthyridin-2-one (25, 150 mg, 0.415 mmol), 2-aminoethanol (250 uL, 253 mg, 4.15 mmol), N,N-diisopropylethylamine (1.0 mL, 742 mg, 5.74 mmol), and 1,4-dioxane (2.0 mL). The reaction vial was placed under N₂, sealed and heated to 150° C. for 17 h. LC/ESI-MS analysis gave a large peak for the desired product with remaining starting material. The reaction was evaporated and purified by normal phase flash column chromatography (SiO₂, 0-10% MeOH in CH₂Cl₂), giving the desired product (26, 98 mg, 61% yield). MS (ESI) [M+H⁺]⁺=387.1.

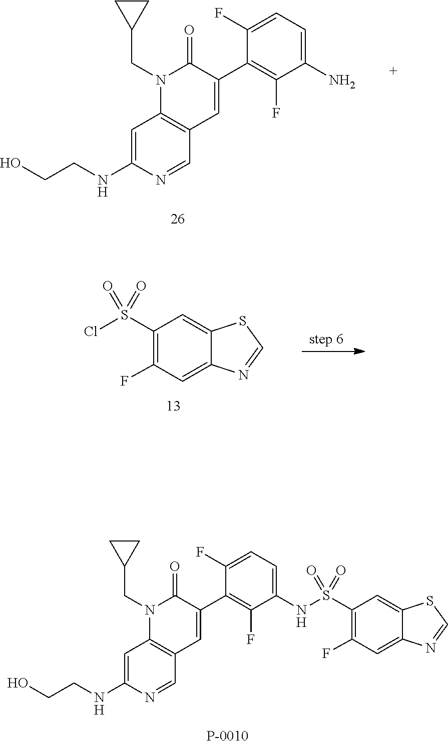 [SMILES: Nc1ccc(F)c(-c2cc3cnc(NCCO)cc3n(CC3CC3)c2=O)c1F ; O=S(=O)(Cl)c1cc2scnc2cc1F ; O=c1c(-c2c(F)ccc(NS(=O)(=O)c3cc4scnc4cc3F)c2F)cc2cnc(NCCO)cc2n1CC1CC1]

Step 6. Preparation of N-(3-(1-(cyclopropylmethyl)-7-((2-hydroxyethyl)amino)-2-oxo-1,2-dihydro-1,6-naphthyridin-3-yl)-2,4-difluorophenyl)-5-fluorobenzo[d]thiazole-6-sulfonamide (P-0010). To a vial containing a stir bar was added 3-(3-amino-2,6-difluoro-phenyl)-1-(cyclopropylmethyl)-7-(2-hydroxyethylamino)-1,6-naphthyridin-2-one (26, 98 mg, 0.254 mmol) and pyridine (1.0 mL, 944 mg, 11.9 mmol). The reaction was placed under N₂ and cooled to 0° C., and 5-fluoro-1,3-benzothiazole-6-sulfonyl chloride (13, 70 mg, 0.278 mmol) was added slowly, in small portions, over 1 m. The reaction was stirred at 0° C. for 1 h, then warmed slowly to 20° C. over 17 h. LC/ESI-MS analysis gave a large peak for the desired product with a small peak for remaining starting material. The reaction was thrice evaporated from toluene (3×10 mL) and purified by normal phase flash column chromatography (SiO₂, 0-15% MeOH in CH₂Cl₂), giving the desired product (P-0010, 15 mg, 9.7% yield). MS (ESI) [M+H⁺]⁺=601.9.

### Example 3

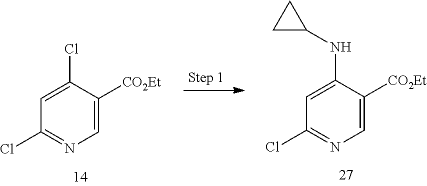 [SMILES: CCOC(=O)c1cnc(Cl)cc1Cl ; CCOC(=O)c1cnc(Cl)cc1NC1CC1]

Step 1. Preparation of ethyl 6-chloro-4-(cyclopropylamino)pyridine-3-carboxylate 27. To a vial containing a stir bar was added ethyl 4,6-dichloropyridine-3-carboxylate (14, 2199 mg, 9.99 mmol) and CH₃CN (10 mL). The reaction was stirred at 20° C., and triethylamine (1533 uL, 1113 mg, 11.0 mmol) was added slowly, dropwise, by micropipettor. To the reaction was added, slowly, dropwise, by micropipettor, cyclopropylmethylamine (763 uL, 629 mg, 11.0 mmol). The reaction was stirred at 20° C. for 1 m, then heated to 50° C. for 17 h. LC/ESI-MS analysis gave a large peak for the desired product with a small peak for remaining starting material. The reaction was added to 5.3 M NH₄Cl (100 mL) and extracted with EtOAc (2×100 mL). The organic fraction was washed with water (1×100 mL) and 5.0 M NaCl (1×100 mL), dried over anhydrous Na₂SO₄, filtered, and evaporated, giving the desired product (27, 2348 mg, 93% yield). MS (ESI) [M+H⁺]⁺=241.0.

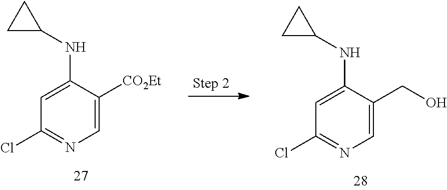 [SMILES: CCOC(=O)c1cnc(Cl)cc1NC1CC1 ; OCc1cnc(Cl)cc1NC1CC1]

Step 2. Preparation of [6-chloro-4-(cyclopropylamino)-3-pyridyl]methanol 28. To a round bottom flask containing a stir bar was added lithium aluminum hydride (1.0 M in THF, 19.5 mL, 0.740 g, 19.5 mmol). The reaction was placed under N₂ and cooled to 0° C., and ethyl 6-chloro-4-(cyclopropylamino)pyridine-3-carboxylate (27, 2348 mg, 9.76 mmol, in THF (10.0 mL)) was added slowly, dropwise, by syringe. Vigorous bubbling was immediately observed upon addition. The reaction was stirred at 0° C. for 1 h, then warmed slowly to 20° C. over 17 h, giving a slightly cloudy pale orange solution. LC/ESI-MS analysis gave a large peak for the desired product with no remaining starting material. The reaction was carefully added to a vigorously stirring solution of Na₂SO₄ decahydrate (31.3 g, 97.3 mmol, in THF (100 mL), 0° C.). The reaction was stirred at 0° C. for 1 m, then warmed to 20° C. over 1 h. The reaction was filtered, dried over anhydrous Na₂SO₄, filtered, and evaporated, giving the desired product (28, 1643 mg, 84% yield).

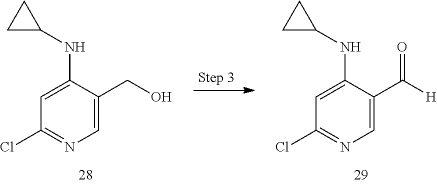 [SMILES: OCc1cnc(Cl)cc1NC1CC1 ; O=Cc1cnc(Cl)cc1NC1CC1]

Step 3. Preparation of 6-chloro-4-(cyclopropylamino)pyridine-3-carbaldehyde 29. To a round bottom flask containing a stir bar was added [6-chloro-4-(cyclopropylamino)-3-pyridyl]methanol (28, 1643 mg, 8.27 mmol), manganese dioxide (activated, 3608 mg, 41.5 mmol), and DCE (82 mL). The reaction was stirred vigorously at 20° C. for 1 m, then heated to 80° C. for 5 h, giving an opaque black solution. LC/ESI-MS analysis gave a large peak for the desired product with no remaining starting material. The reaction was diluted with EtOAc (50 mL), filtered through celite, and evaporated, giving the desired product (29, 1459 mg, 89% yield). MS (ESI) [M+H⁺]⁺=197.1.

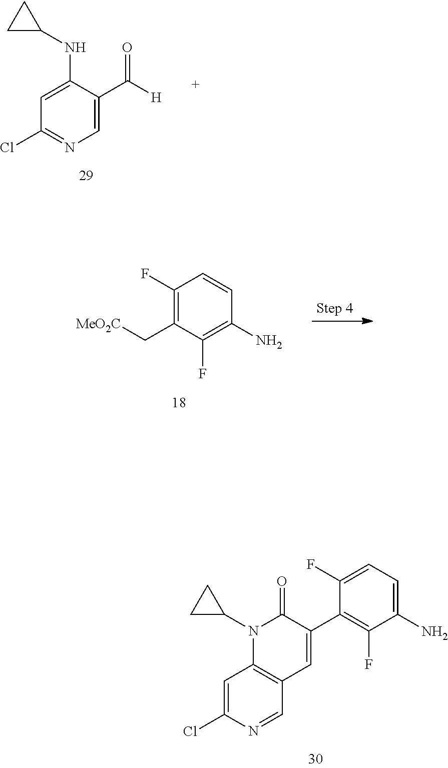 [SMILES: O=Cc1cnc(Cl)cc1NC1CC1 ; COC(=O)Cc1c(F)ccc(N)c1F ; Nc1ccc(F)c(-c2cc3cnc(Cl)cc3n(C3CC3)c2=O)c1F]

Step 4. Preparation of 3-(3-amino-2,6-difluoro-phenyl)-7-chloro-1-cyclopropyl-1,6-naphthyridin-2-one 30. To a round bottom flask containing a stir bar was added 6-chloro-4-(cyclopropylamino)pyridine-3-carbaldehyde (29, 1459 mg, 7.42 mmol), methyl 2-(3-amino-2,6-difluoro-phenyl)acetate (18, 4488 mg, 22.3 mmol), potassium carbonate (6228 mg, 45.1 mmol), and NMP (50 mL). The reaction was placed under N₂ and heated to 120° C. for 3 d. LC/ESI-MS analysis gave a large peak for the desired, cyclized product with no remaining starting material. The reaction was filtered, evaporated, added to 5.3 M NH₄Cl (300 mL), and extracted with EtOAc (2×300 mL). The organic fraction was washed with 5.3 M NH₄Cl (1×100 mL), water (1×100 mL), and 5 M NaCl (1×100 mL), dried over anhydrous Na₂SO₄, filtered, evaporated, and purified by normal phase flash column chromatography (SiO₂, 0-100% EtOAc in hexanes), giving the desired product (30, 953 mg, 37% yield). MS (ESI) [M+H⁺]⁺=348.0.

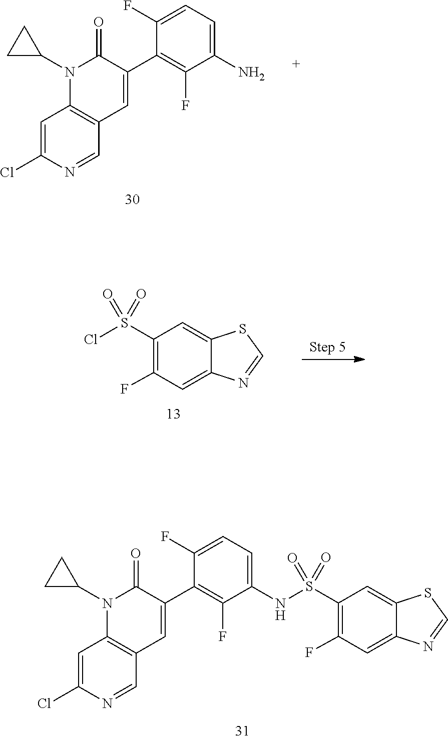 [SMILES: Nc1ccc(F)c(-c2cc3cnc(Cl)cc3n(C3CC3)c2=O)c1F ; O=S(=O)(Cl)c1cc2scnc2cc1F ; O=c1c(-c2c(F)ccc(NS(=O)(=O)c3cc4scnc4cc3F)c2F)cc2cnc(Cl)cc2n1C1CC1]

Step 5. Preparation of N-[3-(7-chloro-1-cyclopropyl-2-oxo-1,6-naphthyridin-3-yl)-2,4-difluoro-phenyl]-5-fluoro-1,3-benzothiazole-6-sulfonamide 31. To a vial containing a stir bar was added 3-(3-amino-2,6-difluoro-phenyl)-7-chloro-1-cyclopropyl-1,6-naphthyridin-2-one (30, 353 mg, 1.01 mmol) and pyridine (5.0 mL, 4.72 g, 59.7 mmol). The reaction was placed under N₂ and cooled to 0° C., and 5-fluoro-1,3-benzothiazole-6-sulfonyl chloride (13, 253 mg, 1.01 mmol) was added slowly, in small portions, over 1 m. The reaction was stirred at 0° C. for 1 h, then warmed slowly to 20° C. over 17 h. LC/ESI-MS analysis gave a large peak for the desired product with a small peak for remaining starting material. The reaction was added to 5.3 M NH₄Cl (100 mL) and extracted with EtOAc (2×100 mL). The organic fraction was washed with water (1×100 mL, containing 12.1 M HCl (5.0 mL, 2.20 g, 60.3 mmol), ending pH ˜0), water (1×100 mL, ending pH ˜5), and 5 M NaCl (1×100 mL, ending pH ˜7), dried over anhydrous Na₂SO₄, filtered, evaporated, and purified by normal phase flash column chromatography (SiO₂, 0-10% MeOH in CH₂Cl₂), giving the desired product (31, 460 mg, 77% yield). MS (ESI) [M+H⁺]⁺=562.8.

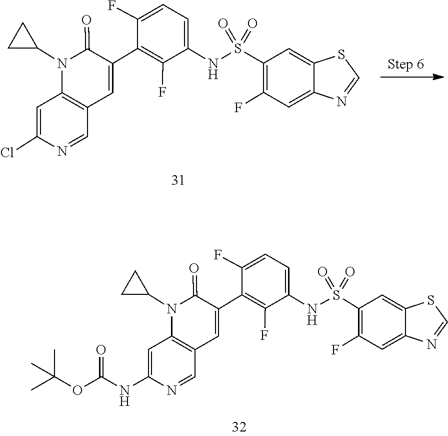 [SMILES: O=c1c(-c2c(F)ccc(NS(=O)(=O)c3cc4scnc4cc3F)c2F)cc2cnc(Cl)cc2n1C1CC1 ; CC(C)(C)OC(=O)Nc1cc2c(cn1)cc(-c1c(F)ccc(NS(=O)(=O)c3cc4scnc4cc3F)c1F)c(=O)n2C1CC1]

Step 6. Preparation of tert-butyl N-[1-cyclopropyl-3-[2,6-difluoro-3-[(5-fluoro-1,3-benzothiazol-6-yl)sulfonylamino]phenyl]-2-oxo-1,6-naphthyridin-7-yl]carbamate 32. To a vial containing a stir bar was added N-[3-(7-chloro-1-cyclopropyl-2-oxo-1,6-naphthyridin-3-yl)-2,4-difluoro-phenyl]-5-fluoro-1,3-benzothiazole-6-sulfonamide (31, 168 mg, 0.30 mmol), tert-butyl carbamate (53 mg, 0.46 mmol), 4,5-bis(diphenylphosphino)-9,9-dimethylxanthene (XantPhos, 35.9 mg, 0.062 mmol), palladium(II) acetate (11.3 mg, 0.050 mmol), cesium carbonate (295 mg, 0.904 mmol), and 1,4-dioxane (3.0 mL). The reaction vessel was placed under N₂, sealed, and heated to 90° C. for 17 h. LC/ESI-MS analysis gave a large peak for the desired product with no remaining starting material. The reaction was added to 5.3 M NH₄Cl (100 mL) and extracted with EtOAc (3×100 mL). The aqueous fraction was found to contain impure desired product as a brownish precipitate that was collected by vacuum filtration. The organic fraction was dried over anhydrous Na₂SO₄, filtered, evaporated, and combined with the previously mentioned brownish precipitate, giving impure desired product (32, 240 mg, 91% yield). MS (ESI) [M+H⁺]⁺=643.9.

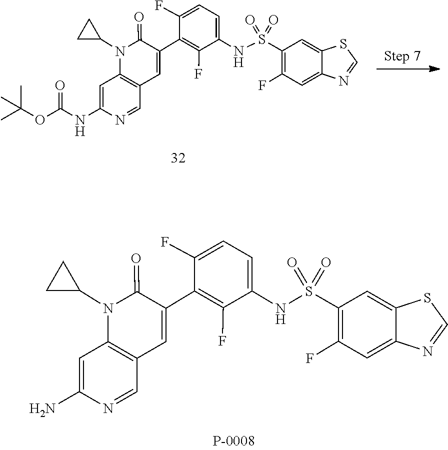 [SMILES: CC(C)(C)OC(=O)Nc1cc2c(cn1)cc(-c1c(F)ccc(NS(=O)(=O)c3cc4scnc4cc3F)c1F)c(=O)n2C1CC1 ; Nc1cc2c(cn1)cc(-c1c(F)ccc(NS(=O)(=O)c3cc4scnc4cc3F)c1F)c(=O)n2C1CC1]

Step 7. Preparation of N-(3-(7-amino-1-cyclopropyl-2-oxo-1,2-dihydro-1,6-naphthyridin-3-yl)-2,4-difluorophenyl)-5-fluorobenzo[d]thiazole-6-sulfonamide (P-0008). To a vial containing a stir bar was added impure tert-butyl N-[1-cyclopropyl-3-[2,6-difluoro-3-[(5-fluoro-1,3-benzothiazol-6-yl)sulfonylamino]phenyl]-2-oxo-1,6-naphthyridin-7-yl]carbamate (32, 240 mg, 0.271 mmol), trifluoroacetic acid (5.0 mL, 7.45 g, 65.3 mmol), and water (0.25 mL, 0.25 g, 13.9 mmol). The reaction was stirred at 20° C. for 1 h. LC/ESI-MS analysis gave a large peak for the desired product with no remaining starting material. The reaction was evaporated and purified by reverse phase flash column chromatography (C18, 0-100% CH₃CN (0.1% HCO₂H), water (0.1% HCO2H)), giving the desired product (P-0008, 78 mg, 53% yield). MS (ESI) [M+H⁺]⁺=543.9.

### Example 4

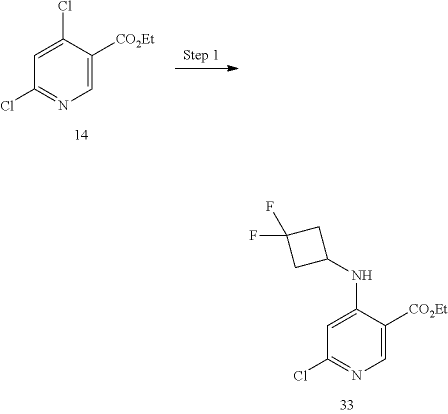 [SMILES: CCOC(=O)c1cnc(Cl)cc1Cl ; CCOC(=O)c1cnc(Cl)cc1NC1CC(F)(F)C1]

Step 1. Preparation of ethyl 6-chloro-4-[(3,3-difluorocyclobutyl)amino]pyridine-3-carboxylate 33. To a vial containing a stir bar was added ethyl 4,6-dichloropyridine-3-carboxylate (14, 2201 mg, 10.0 mmol), 3,3-difluorocyclobutanamine hydrochloride (1585 mg, 11.0 mmol) and CH₃CN (10.0 mL). The reaction was stirred at 20° C., and triethylamine (3066 uL, 2226 mg, 22.0 mmol) was added slowly, dropwise, by micropipettor. The reaction was stirred at 20° C. for 1 m, then heated to 50° C. for 2 d. LC/ESI-MS analysis gave a large peak for the desired product with a small peak for remaining starting material. The reaction was added to 5.3 M NH₄Cl (100 mL) and extracted with EtOAc (2×100 mL). The organic fraction was washed with water (1×100 mL) and 5.0 M NaCl (1×100 mL), dried over anhydrous Na₂SO₄, filtered, and evaporated, giving the desired product (33, 2754 mg, 88% yield). MS (ESI) [M+H⁺]⁺=291.0.

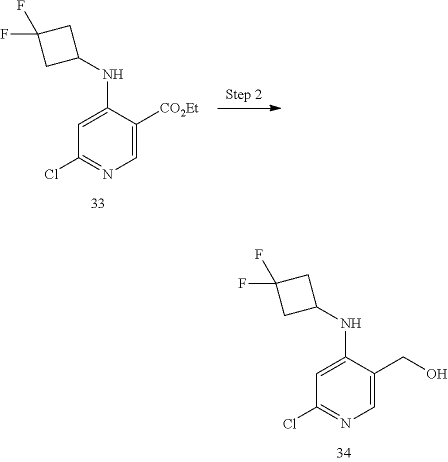 [SMILES: CCOC(=O)c1cnc(Cl)cc1NC1CC(F)(F)C1 ; OCc1cnc(Cl)cc1NC1CC(F)(F)C1]

Step 2. Preparation of [6-chloro-4-[(3,3-difluorocyclobutyl)amino]-3-pyridyl]methanol 34. To a round bottom flask containing a stir bar was added lithium aluminum hydride (1.0 M in THF, 19.0 mL, 0.721 g, 19.0 mmol). The reaction was placed under N₂ and cooled to 0° C., and ethyl 6-chloro-4-[(3,3-difluorocyclobutyl)amino]pyridine-3-carboxylate (33, 2754 mg, 9.47 mmol, in THF (10.0 mL)) was added slowly, dropwise, by syringe. Vigorous bubbling was immediately observed upon addition. The reaction was stirred at 0° C. for 1 h, then warmed slowly to 20° C. over 17 h, giving a slightly cloudy pale orange solution. LC/ESI-MS analysis gave a large peak for the desired product with no remaining starting material. The reaction was carefully added to a vigorously stirring solution of Na₂SO₄ decahydrate (31.2 g, 96.8 mmol, in THF (100 mL), 0° C.). The reaction was stirred at 0° C. for 1 m, then warmed to 20° C. over 1 h. The reaction was filtered, dried over anhydrous Na₂SO₄, filtered, and evaporated, giving the desired product (34, 1665 mg, 70.0% yield).

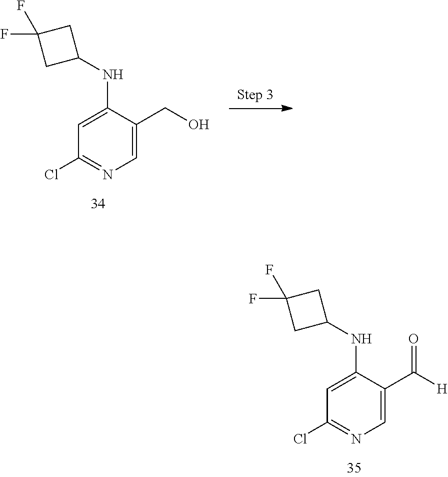 [SMILES: OCc1cnc(Cl)cc1NC1CC(F)(F)C1 ; O=Cc1cnc(Cl)cc1NC1CC(F)(F)C1]

Step 3. Preparation of 6-chloro-4-[(3,3-difluorocyclobutyl)amino]pyridine-3-carbaldehyde 35. To a round bottom flask containing a stir bar was added [6-chloro-4-[(3,3-difluorocyclobutyl)amino]-3-pyridyl]methanol (34, 1665 mg, 6.70 mmol), manganese dioxide (activated, 2875 mg, 33.1 mmol), and DCE (66.0 mL). The reaction was stirred vigorously at 20° C. for 1 m, then heated to 80° C. for 4 h, giving an opaque black solution. LC/ESI-MS analysis gave a large peak for the desired product with a small peak for remaining starting material. To the reaction was added additional manganese dioxide (579 mg, 6.66 mmol). The reaction was heated at 80° C. for an additional 1 h, giving an opaque black solution. LC/ESI-MS analysis gave a large peak for the desired product with no remaining starting material. The reaction was diluted with EtOAc (50 mL), filtered through celite, and evaporated, giving the desired product (35, 1395 mg, 83% yield). MS (ESI) [M+H⁺]⁺=247.0.

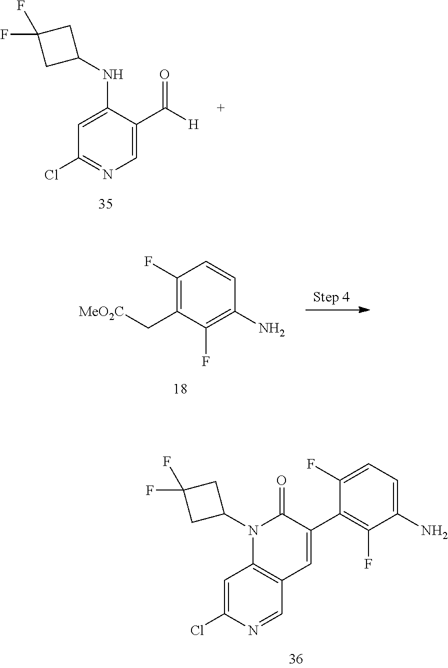 [SMILES: O=Cc1cnc(Cl)cc1NC1CC(F)(F)C1 ; COC(=O)Cc1c(F)ccc(N)c1F ; Nc1ccc(F)c(-c2cc3cnc(Cl)cc3n(C3CC(F)(F)C3)c2=O)c1F]

Step 4. Preparation of 3-(3-amino-2,6-difluoro-phenyl)-7-chloro-1-(3,3-difluorocyclobutyl)-1,6-naphthyridin-2-one 36. To a round bottom flask containing a stir bar was added 6-chloro-4-[(3,3-difluorocyclobutyl)amino]pyridine-3-carbaldehyde (35, 1395 mg, 5.66 mmol), methyl 2-(3-amino-2,6-difluoro-phenyl)acetate (18, 3422 mg, 17.0 mmol), potassium carbonate (4705 mg, 34.0 mmol), and NMP (50.0 mL). The reaction was placed under N₂ and heated to 120° C. for 3 d. LC/ESI-MS analysis gave a large peak for the desired, cyclized product with no remaining starting material. The reaction was filtered, evaporated, added to 5.3 M NH₄Cl (300 mL), and extracted with EtOAc (2×300 mL). The organic fraction was washed with 5.3 M NH₄Cl (1×100 mL), water (1×100 mL), and 5 M NaCl (1×100 mL), dried over anhydrous Na₂SO₄, filtered, evaporated, and purified by normal phase flash column chromatography (SiO₂, 0-100% EtOAc in hexanes), giving the desired product (36, 780 mg, 35% yield). MS (ESI) [M+H⁺]⁺=398.0.

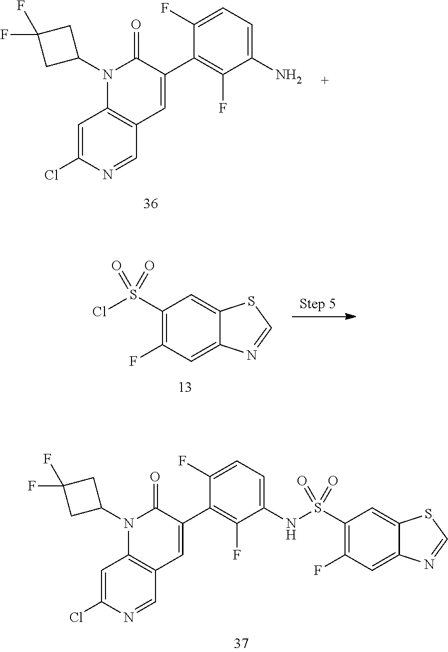 [SMILES: Nc1ccc(F)c(-c2cc3cnc(Cl)cc3n(C3CC(F)(F)C3)c2=O)c1F ; O=S(=O)(Cl)c1cc2scnc2cc1F ; O=c1c(-c2c(F)ccc(NS(=O)(=O)c3cc4scnc4cc3F)c2F)cc2cnc(Cl)cc2n1C1CC(F)(F)C1]

Step 5. Preparation of N-[3-[7-chloro-1-(3,3-difluorocyclobutyl)-2-oxo-1,6-naphthyridin-3-yl]-2,4-difluoro-phenyl]-5-fluoro-1,3-benzothiazole-6-sulfonamide 37. To a vial containing a stir bar was added 3-(3-amino-2,6-difluoro-phenyl)-7-chloro-1-(3,3-difluorocyclobutyl)-1,6-naphthyridin-2-one (36, 399 mg, 1.00 mmol) and pyridine (5.0 mL, 4.72 g, 59.7 mmol). The reaction was placed under N₂ and cooled to 0° C., and 5-fluoro-1,3-benzothiazole-6-sulfonyl chloride (13, 252 mg, 1.00 mmol) was added slowly, in small portions, over 1 m. The reaction was stirred at 0° C. for 1 h, then warmed slowly to 20° C. over 17 h. LC/ESI-MS analysis gave a large peak for the desired product with a small peak for remaining starting material. The reaction was added to 5.3 M NH₄Cl (100 mL) and extracted with EtOAc (2×100 mL). The organic fraction was washed with water (1×100 mL, containing 12.06 M HCl (5.0 mL, 2.20 g, 60.3 mmol), ending pH ˜0), water (1×100 mL, ending pH ˜5), and 5 M NaCl (1×100 mL, ending pH ˜7), dried over anhydrous Na₂SO₄, filtered, evaporated, and purified by normal phase flash column chromatography (SiO₂, 0-10% MeOH in CH₂Cl₂), giving the desired product (37, 521 mg, 76% yield). MS (ESI) [M+H⁺]⁺=612.8.

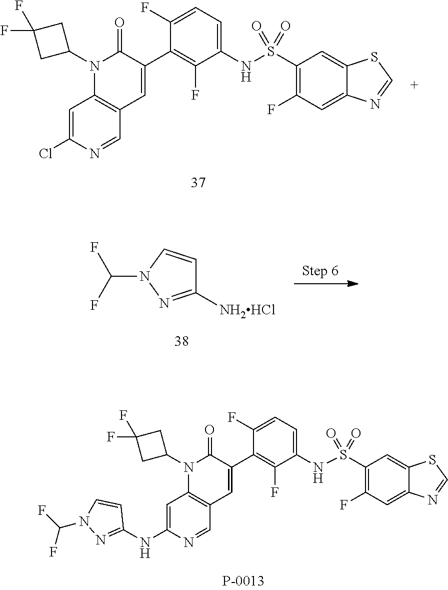

Step 6. Preparation of N-(3-(1-(3,3-difluorocyclobutyl)-7-((1-(difluoromethyl)-1H-pyrazol-3-yl)amino)-2-oxo-1,2-dihydro-1,6-naphthyridin-3-yl)-2,4-difluorophenyl)-5-fluorobenzo[d]thiazole-6-sulfonamide (P-0013). To a vial containing a stir bar was added N-[3-[7-chloro-1-(3,3-difluorocyclobutyl)-2-oxo-1,6-naphthyridin-3-yl]-2,4-difluoro-phenyl]-5-fluoro-1,3-benzothiazole-6-sulfonamide (37, 184 mg, 0.301 mmol), 1-(difluoromethyl)pyrazol-3-amine hydrochloride (38, 79.7 mg, 0.470 mmol), 4,5-bis(diphenylphosphino)-9,9-dimethylxanthene (XantPhos, 38.5 mg, 0.067 mmol), palladium(II) acetate (11.9 mg, 0.053 mmol), cesium carbonate (494 mg, 1.52 mmol), and 1,4-dioxane (5.0 mL). The reaction vessel was placed under N₂, sealed, and heated to 90° C. for 17 h. LC/ESI-MS analysis gave a large peak for the desired product with no remaining starting material. The reaction was added to 5.3 M NH₄Cl (100 mL) and extracted with EtOAc (2×100 mL). The organic fraction was washed with water (1×100 mL) and 5 M NaCl (1×100 mL), dried over anhydrous Na₂SO₄, filtered, evaporated, purified by normal phase flash column chromatography (SiO₂, 0-10% MeOH in CH₂Cl₂), and further purified by normal phase flash column chromatography (SiO₂, 0-100% EtOAc in hexanes), giving the desired product (P-0013, 80.5 mg, 36% yield). MS (ESI) [M+H⁺]⁺=709.9.

### Example 5

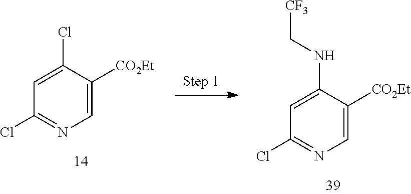 [SMILES: CCOC(=O)c1cnc(Cl)cc1Cl ; CCOC(=O)c1cnc(Cl)cc1NCC(F)(F)F]

Step 1. Preparation of ethyl 6-chloro-4-(2,2,2-trifluoroethylamino)pyridine-3-carboxylate 39. To a pressure vessel containing a stir bar was added ethyl 4,6-dichloropyridine-3-carboxylate (14, 2201 mg, 10.0 mmol), 2,2,2-trifluoroethanamine (4953 mg, 50.0 mmol), N,N-diisopropylethylamine (8.0 mL, 5936 mg, 45.9 mmol), and 1,4-dioxane (16.0 mL). The reaction was placed under N₂, sealed, and heated to 130° C. for 3 d. LC/ESI-MS analysis gave a large peak for the desired product with remaining starting material. The reaction was filtered, evaporated, and purified by normal phase flash column chromatography (SiO₂, 0-50% EtOAc in hexanes) giving the desired product (39, 860 mg, 30% yield). MS (ESI) [M+H⁺]⁺=282.9.

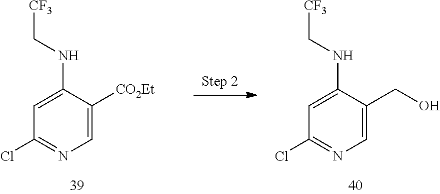 [SMILES: CCOC(=O)c1cnc(Cl)cc1NCC(F)(F)F ; OCc1cnc(Cl)cc1NCC(F)(F)F]

Step 2. Preparation of [6-chloro-4-(2,2,2-trifluoroethylamino)-3-pyridyl]methanol 40. To a round bottom flask containing a stir bar was added ethyl 6-chloro-4-(2,2,2-trifluoroethylamino)pyridine-3-carboxylate (39, 860 mg, 3.04 mmol) and THF (20 mL). The reaction was placed under N₂ and cooled to 0° C., and lithium aluminum hydride (2.0 M in THF, 3.35 mL, 6.70 mmol) was added slowly, dropwise, by syringe. Vigorous bubbling was immediately observed upon addition. The reaction was stirred at 0° C. for 1 h, then warmed slowly to 20° C. over 1 h. LC/ESI-MS analysis gave a large peak for the desired product with no remaining starting material. The reaction was quenched with water (127 uL, 127 mg, 7.06 mmol), NaOH (4.0 M, 84 uL, 0.34 mmol), and water (381 uL, 381 mg, 21.2 mmol). The reaction was diluted with CH₂Cl₂ (100 mL), dried over MgSO₄, filtered, and evaporated, giving the desired product (40, 732 mg, 97% yield). MS (ESI) [M+H⁺]⁺=241.0.

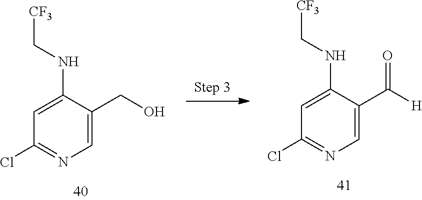 [SMILES: OCc1cnc(Cl)cc1NCC(F)(F)F ; O=Cc1cnc(Cl)cc1NCC(F)(F)F]

Step 3. Preparation of 6-chloro-4-(2,2,2-trifluoroethylamino)pyridine-3-carbaldehyde 41. To a round bottom flask containing a stir bar was added [6-chloro-4-(2,2,2-trifluoroethylamino)-3-pyridyl]methanol (40, 732 mg, 3.04 mmol), manganese dioxide (529 mg, 6.08 mmol), and EtOAc (30 mL). The reaction was heated to 80° C. for 2 h. LC/ESI-MS analysis showed a large peak for the desired product with remaining starting material. To the reaction was added additional manganese dioxide (529 mg, 6.08 mmol). The reaction was heated to 80° C. for an additional 4 h. LC/ESI-MS analysis gave a large peak for the desired product with no remaining starting material. The reaction was filtered through celite and evaporated, giving the desired product (41, 650 mg, 90% yield). MS (ESI) [M+H⁺]⁺=238.9.

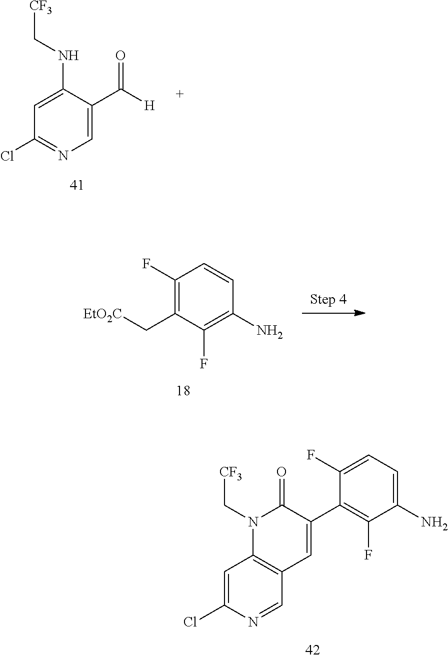 [SMILES: O=Cc1cnc(Cl)cc1NCC(F)(F)F ; CCOC(=O)Cc1c(F)ccc(N)c1F ; Nc1ccc(F)c(-c2cc3cnc(Cl)cc3n(CC(F)(F)F)c2=O)c1F]

Step 4. Preparation of 3-(3-amino-2,6-difluoro-phenyl)-7-chloro-1-(2,2,2-trifluoroethyl)-1,6-naphthyridin-2-one 42. To a pressure vessel containing a stir bar was added 6-chloro-4-(2,2,2-trifluoroethylamino)pyridine-3-carbaldehyde (41, 649 mg, 2.72 mmol), methyl 2-(3-amino-2,6-difluoro-phenyl)acetate (18, 821 mg, 4.08 mmol), potassium carbonate (1128 mg, 8.16 mmol), and DMF (15.0 mL). The reaction was placed under N₂, sealed, and heated to 120° C. for 17 h. LC/ESI-MS analysis gave a large peak for the desired, cyclized product with no remaining starting material. The reaction was added to water (100 mL), and extracted with EtOAc (2×100 mL). The organic fraction was washed with water (1×100 mL), and 5 M NaCl (1×100 mL), dried over anhydrous Na₂SO₄, filtered, evaporated, and purified by normal phase flash column chromatography (SiO₂, 0-50% EtOAc in hexanes), giving the desired product (42, 265 mg, 25% yield). MS (ESI) [M+H⁺]⁺=390.0.

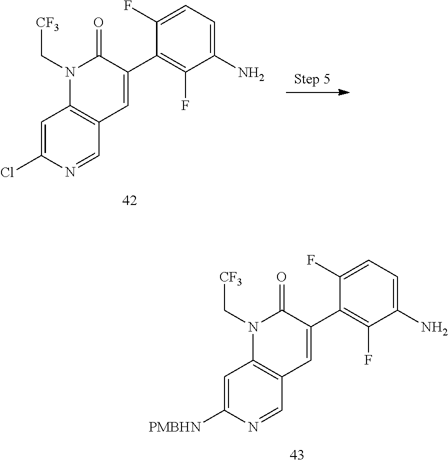

Step 5. Preparation of 3-(3-amino-2,6-difluorophenyl)-7-((4-methoxybenzyl)amino)-1-(2,2,2-trifluoroethyl)-1,6-naphthyridin-2(1H)-one 43. To a vial containing a stir bar was added 3-(3-amino-2,6-difluoro-phenyl)-7-chloro-1-(2,2,2-trifluoroethyl)-1,6-naphthyridin-2-one (42, 265 mg, 0.680 mmol), (4-methoxyphenyl)methanamine (355 uL, 373 mg, 2.72 mmol), N,N-diisopropylethylamine (1.0 mL, 742 mg, 5.74 mmol), and 1,4-dioxane (2.0 mL). The reaction vial was placed under N₂, sealed, and heated to 155° C. for 3 d. LC/ESI-MS analysis gave a large peak for the desired product with a very small peak for remaining starting material. The reaction was evaporated and precipitated from EtOAc (˜5 mL), giving the desired product (43, 207 mg, 62% yield). MS (ESI) [M+H⁺]⁺=491.0.

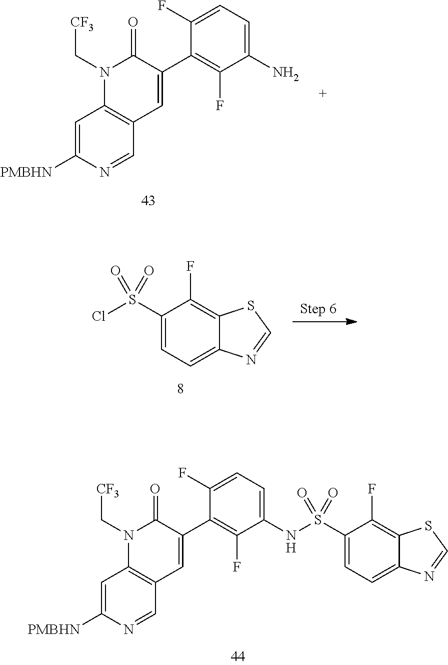

Step 6. Preparation of N-(2,4-difluoro-3-(7-((4-methoxybenzyl)amino)-2-oxo-1-(2,2,2-trifluoroethyl)-1,2-dihydro-1,6-naphthyridin-3-yl)phenyl)-7-fluorobenzo[d]thiazole-6-sulfonamide 44. To a vial containing a stir bar was added 3-(3-amino-2,6-difluorophenyl)-7-((4-methoxybenzyl)amino)-1-(2,2,2-trifluoroethyl)-1,6-naphthyridin-2(1H)-one (43, 103 mg, 0.210 mmol) and pyridine (1.0 mL, 944 mg, 11.9 mmol). The reaction was placed under N₂ and cooled to 0° C., and 7-fluoro-1,3-benzothiazole-6-sulfonyl chloride (8, 79 mg, 0.315 mmol) was added slowly, in small portions, over 1 m. The reaction was stirred at 0° C. for 1 h, then warmed slowly to 20° C. over 3 h. LC/ESI-MS analysis gave a large peak for the desired product with no remaining starting material. The reaction was evaporated, dissolved in EtOAc (100 mL), washed with 0.5 M HCl (1×100 mL), water (1×100 mL), and 5 M NaCl (1×100 mL), dried over anhydrous Na₂SO₄, filtered, evaporated, and purified by normal phase flash column chromatography (SiO₂, 0-5% MeOH in EtOAc), giving the desired product (44, 73 mg, 49% yield). MS (ESI) [M+H⁺]⁺=705.9.

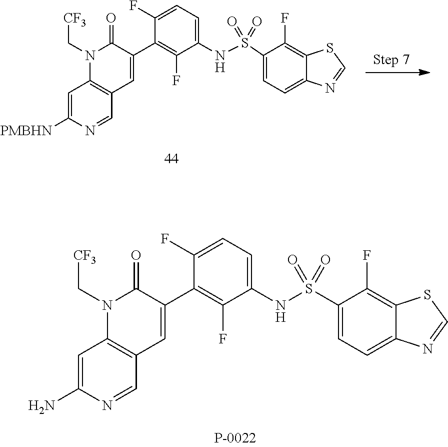

Step 7. Preparation of N-(3-(7-amino-2-oxo-1-(2,2,2-trifluoroethyl)-1,2-dihydro-1,6-naphthyridin-3-yl)-2,4-difluorophenyl)-7-fluorobenzo[d]thiazole-6-sulfonamide (P-0022). To a vial containing a stir bar was added N-(2,4-difluoro-3-(7-((4-methoxybenzyl)amino)-2-oxo-1-(2,2,2-trifluoroethyl)-1,2-dihydro-1,6-naphthyridin-3-yl)phenyl)-7-fluorobenzo[d]thiazole-6-sulfonamide (44, 73 mg, 0.103 mmol), trifluoroacetic acid (500 uL, 745 mg, 6.53 mmol), and CH₂Cl₂ (1.5 mL). The reaction was placed under N₂, sealed, and heated to 65° C. for 4 h. LC/ESI-MS analysis gave a large peak for the desired product with no remaining starting material. The reaction was evaporated and purified by reverse phase flash column chromatography (C18, 0-100% CH₃CN (0.1% HCo2H), water (0.1% HCO2H)), giving the desired product (P-0022, 44 mg, 72% yield). MS (ESI) [M+H⁺]⁺=585.9.

All compounds in Table 1 listed below can be made according to the synthetic examples described in this disclosure, and by making any necessary substitutions of starting materials that the skilled artisan would be able to obtain either commercially or otherwise.

TABLE 1

| P# | Structure | Name | (MH)+ |
|---|---|---|---|
| P-0001 | 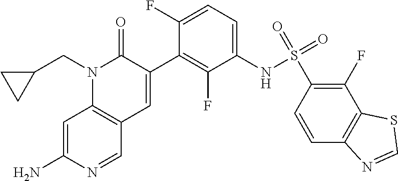 [SMILES: Nc1cc2c(cn1)cc(-c1c(F)ccc(NS(=O)(=O)c3ccc4ncsc4c3F)c1F)c(=O)n2CC1CC1] | N-(3-(7-amino-1- (cyclopropylmethyl)-2-oxo- 1,2-dihydro-1,6- naphthyridin-3-yl)-2,4- difluorophenyl)-7- fluorobenzo[d]thiazole-6- sulfonamide | 557.9 |
| P-0002 | 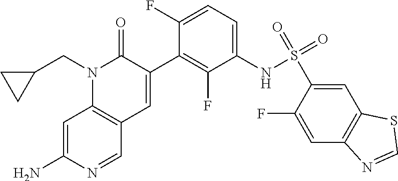 [SMILES: Nc1cc2c(cn1)cc(-c1c(F)ccc(NS(=O)(=O)c3cc4scnc4cc3F)c1F)c(=O)n2CC1CC1] | N-(3-(7-amino-1- (cyclopropylmethyl)-2-oxo- 1,2-dihydro-1,6- naphthyridin-3-yl)-2,4- difluorophenyl)-5- fluorobenzo[d]thiazole-6- sulfonamide | 557.9 |
| P-0003 | 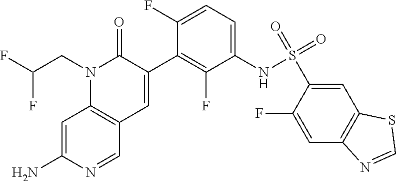 [SMILES: Nc1cc2c(cn1)cc(-c1c(F)ccc(NS(=O)(=O)c3cc4scnc4cc3F)c1F)c(=O)n2CC(F)F] | N-(3-(7-amino-1-(2,2- difluoroethyl)-2-oxo-1,2- dihydro-1,6-naphthyridin-3- yl)-2,4-difluorophenyl)-5- fluorobenzo[d]thiazole-6- sulfonamide | 567.9 |
| P-0004 | 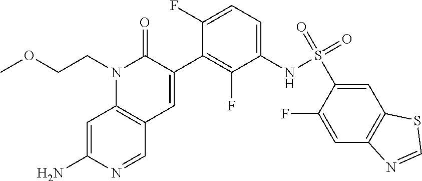 [SMILES: COCCn1c(=O)c(-c2c(F)ccc(NS(=O)(=O)c3cc4scnc4cc3F)c2F)cc2cnc(N)cc21] | N-(3-(7-amino-1-(2- methoxyethyl)-2-oxo-1,2- dihydro-1,6-naphthyridin-3- yl)-2,4-difluorophenyl)-5- fluorobenzo[d]thiazole-6- sulfonamide | 561.9 |
| P-0005 | 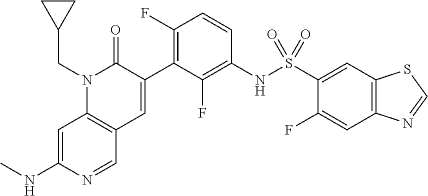 [SMILES: CNc1cc2c(cn1)cc(-c1c(F)ccc(NS(=O)(=O)c3cc4scnc4cc3F)c1F)c(=O)n2CC1CC1] | N-(3-(1- (cyclopropylmethyl)-7- (methylamino)-2-oxo-1,2- dihydro-1,6-naphthyridin-3- yl)-2,4-difluorophenyl)-5- fluorobenzo[d]thiazole-6- sulfonamide | 571.9 |
| P-0006 | 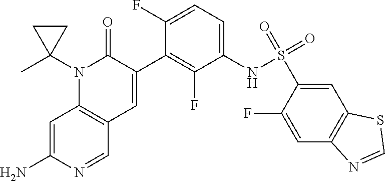 [SMILES: CC1(n2c(=O)c(-c3c(F)ccc(NS(=O)(=O)c4cc5scnc5cc4F)c3F)cc3cnc(N)cc32)CC1] | N-(3-(7-amino-1-(1- methylcyclopropyl)-2-oxo- 1,2-dihydro-1,6- naphthyridin-3-yl)-2,4- difluorophenyl)-5- fluorobenzo[d]thiazole-6- sulfonamide | 557.9 |
| P-0007 | 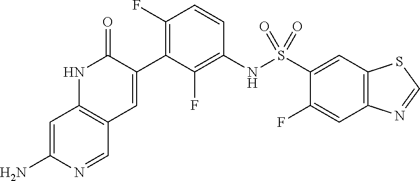 [SMILES: Nc1cc2[nH]c(=O)c(-c3c(F)ccc(NS(=O)(=O)c4cc5scnc5cc4F)c3F)cc2cn1] | N-(3-(7-amino-2-oxo-1,2- dihydro-1,6-naphthyridin-3- yl)-2,4-difluorophenyl)-5- fluorobenzo[d]thiazole-6- sulfonamide | 503.9 |
| P-0008 | 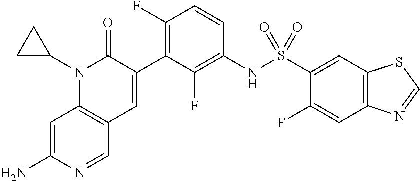 [SMILES: Nc1cc2c(cn1)cc(-c1c(F)ccc(NS(=O)(=O)c3cc4scnc4cc3F)c1F)c(=O)n2C1CC1] | N-(3-(7-amino-1- cyclopropyl-2-oxo-1,2- dihydro-1,6-naphthyridin-3- yl)-2,4-difluorophenyl)-5- fluorobenzo[d]thiazole-6- sulfonamide | 543.9 |
| P-0009 | 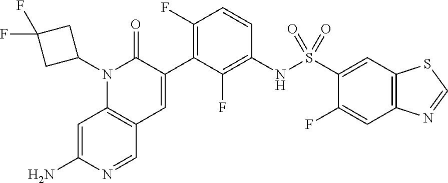 [SMILES: Nc1cc2c(cn1)cc(-c1c(F)ccc(NS(=O)(=O)c3cc4scnc4cc3F)c1F)c(=O)n2C1CC(F)(F)C1] | N-(3-(7-amino-1-(3,3- difluorocyclobutyl)-2-oxo- 1,2-dihydro-1,6- naphthyridin-3-yl)-2,4- difluorophenyl)-5- fluorobenzo[d]thiazole-6- sulfonamide | 593.9 |
| P-0010 | 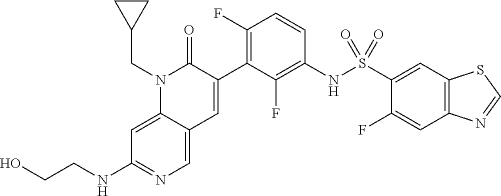 [SMILES: O=c1c(-c2c(F)ccc(NS(=O)(=O)c3cc4scnc4cc3F)c2F)cc2cnc(NCCO)cc2n1CC1CC1] | N-(3-(1- (cyclopropylmethyl)-7-((2- hydroxyethyl)amino)-2-oxo- 1,2-dihydro-1,6- naphthyridin-3-yl)-2,4- difluorophenyl)-5- fluorobenzo[d]thiazole-6- sulfonamide | 601.9 |
| P-0011 | 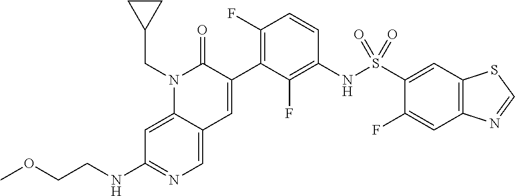 [SMILES: COCCNc1cc2c(cn1)cc(-c1c(F)ccc(NS(=O)(=O)c3cc4scnc4cc3F)c1F)c(=O)n2CC1CC1] | N-(3-(1- (cyclopropylmethyl)-7-((2- methoxyethyl)amino)-2-oxo- 1,2-dihydro-1,6- naphthyridin-3-yl)-2,4- difluorophenyl)-5- fluorobenzo[d]thiazole-6- sulfonamide | 615.9 |
| P-0012 | 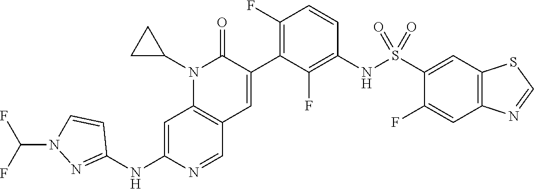 [SMILES: O=c1c(-c2c(F)ccc(NS(=O)(=O)c3cc4scnc4cc3F)c2F)cc2cnc(Nc3ccn(C(F)F)n3)cc2n1C1CC1] | N-(3-(1-cyclopropyl-7-((1- (difluoromethyl)-1H- pyrazol-3-yl)amino)-2-oxo- 1,2-dihydro-1,6- naphthyndin-3-yl)-2,4- difluorophenyl)-5- fluorobenzo[d]thiazole-6- sulfonamide | 659.9 |
| P-0013 | 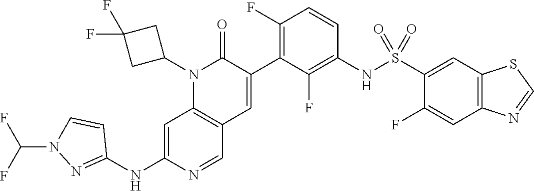 [SMILES: O=c1c(-c2c(F)ccc(NS(=O)(=O)c3cc4scnc4cc3F)c2F)cc2cnc(Nc3ccn(C(F)F)n3)cc2n1C1CC(F)(F)C1] | N-(3-(1-(3,3- difluorocyclobutyl)-7-((1- (difluoromethyl)-1H- pyrazol-3-yl)amino)-2-oxo- 1,2-dihydro-1,6- naphthyridin-3-yl)-2,4- difluorophenyl)-5- fluorobenzo[d]thiazole-6- sulfonamide | 709.9 |
| P-0014 | 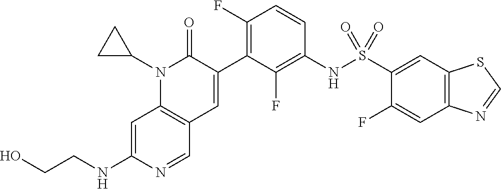 [SMILES: O=c1c(-c2c(F)ccc(NS(=O)(=O)c3cc4scnc4cc3F)c2F)cc2cnc(NCCO)cc2n1C1CC1] | N-(3-(1-cyclopropyl-7-((2- hydroxyethyl)amino)-2-oxo- 1,2-dihydro-1,6- naphthyridin-3-yl)-2,4- difluorophenyl)-5- fluorobenzo[d]thiazole-6- sulfonamide | 587.9 |
| P-0015 | 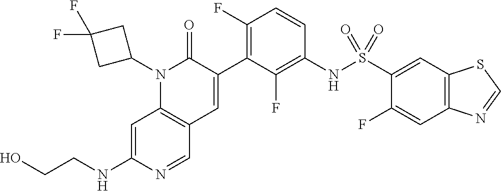 [SMILES: O=c1c(-c2c(F)ccc(NS(=O)(=O)c3cc4scnc4cc3F)c2F)cc2cnc(NCCO)cc2n1C1CC(F)(F)C1] | N-(3-(1-(3,3- difluorocyclobutyl)-7-((2- hydroxyethyl)amino)-2-oxo- 1,2-dihydro-1,6- naphthyridin-3-yl)-2,4- difluorophenyl)-5- fluorobenzo[d]thiazole-6- sulfonamide | 637.9 |
| P-0016 | 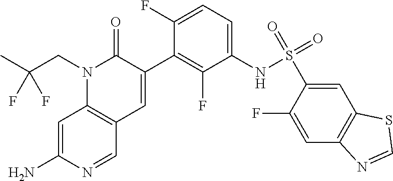 [SMILES: CC(F)(F)Cn1c(=O)c(-c2c(F)ccc(NS(=O)(=O)c3cc4scnc4cc3F)c2F)cc2cnc(N)cc21] | N-(3-(7-amino-1-(2,2- difluoropropyl)-2-oxo-1,2- dihydro-1,6-naphthyridin-3- yl)-2,4-difluorophenyl)-5- fluorobenzo[d]thiazole-6- sulfonamide | 581.9 |
| P-0017 | 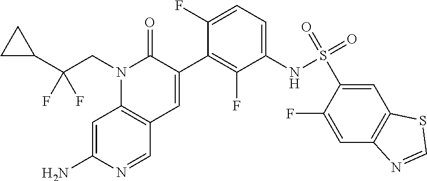 [SMILES: Nc1cc2c(cn1)cc(-c1c(F)ccc(NS(=O)(=O)c3cc4scnc4cc3F)c1F)c(=O)n2CC(F)(F)C1CC1] | N-(3-(7-amino-1-(2- cyclopropyl-2,2- difluoroethyl)-2-oxo-1,2- dihydro-1,6-naphthyridin-3- yl)-2,4-difluorophenyl)-5- fluorobenzo[d]thiazole-6- sulfonnamide | 607.9 |
| P-0018 | 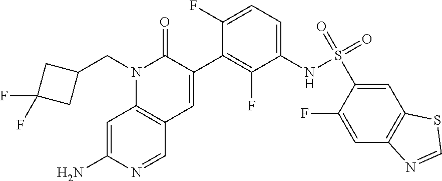 [SMILES: Nc1cc2c(cn1)cc(-c1c(F)ccc(NS(=O)(=O)c3cc4scnc4cc3F)c1F)c(=O)n2CC1CC(F)(F)C1] | N-(3-(7-amino-1-((3,3- difluorocyclobutyl)methyl)- 2-oxo-1,2-dihydro-1,6- naphthyridin-3-yl)-2,4- difluorophenyl)-5- fluorobenzo[d]thiazole-6- sulfonamide | 607.9 |
| P-0019 | 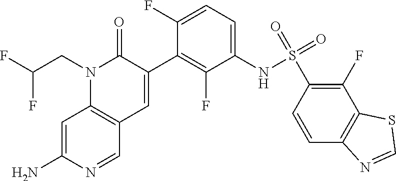 [SMILES: Nc1cc2c(cn1)cc(-c1c(F)ccc(NS(=O)(=O)c3ccc4ncsc4c3F)c1F)c(=O)n2CC(F)F] | N-(3-(7-amino-1-(2,2- difluoroethyl)-2-oxo-1,2- dihydro-1,6-naphthyridin-3- yl)-2,4-difluorophenyl)-7- fluorobenzo[d]thiazole-6- sulfonamide | 567.9 |
| P-0020 | 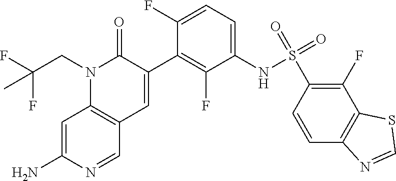 [SMILES: CC(F)(F)Cn1c(=O)c(-c2c(F)ccc(NS(=O)(=O)c3ccc4ncsc4c3F)c2F)cc2cnc(N)cc21] | N-(3-(7-amino-1-(2,2- difluoropropyl)-2-oxo-1,2- dihydro-1,6-naphthyridin-3- yl)-2,4-difluorophenyl)-7- fluorobenzo[d]thiazole-6- sulfonamide | 581.9 |
| P-0021 | 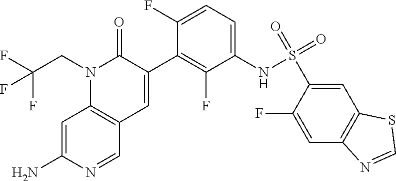 [SMILES: Nc1cc2c(cn1)cc(-c1c(F)ccc(NS(=O)(=O)c3cc4scnc4cc3F)c1F)c(=O)n2CC(F)(F)F] | N-(3-(7-amino-2-oxo-1- (2,2,2-trifluoroethyl)-1,2- dihydro-1,6-naphthyridin-3- yl)-2,4-difluorophenyl)-5- fluorobenzo[d]thiazole-6- sulfonamide | 585.9 |
| P-0022 | 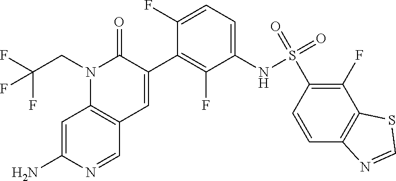 [SMILES: Nc1cc2c(cn1)cc(-c1c(F)ccc(NS(=O)(=O)c3ccc4ncsc4c3F)c1F)c(=O)n2CC(F)(F)F] | N-(3-(7-amino-2-oxo-1- (2,2,2-trifluoroethyl)-1,2- dihydro-1,6-naphthyridin-3- yl)-2,4-difluorophenyl)-7- fluorobenzo[d]thiazole-6- sulfonamide | 585.9 |
| P-0023 | 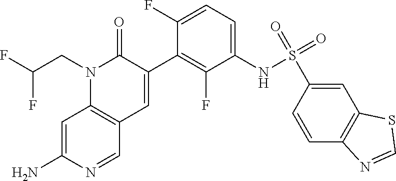 [SMILES: Nc1cc2c(cn1)cc(-c1c(F)ccc(NS(=O)(=O)c3ccc4ncsc4c3)c1F)c(=O)n2CC(F)F] | N-(3-(7-amino-1-(2,2- difluoroethyl)-2-oxo-1,2- dihydro-1,6-naphthyridin-3- yl)-2,4- difluorophenyl)benzo[d] thiazole-6-sulfonamide | 549.9 |
| P-0024 | 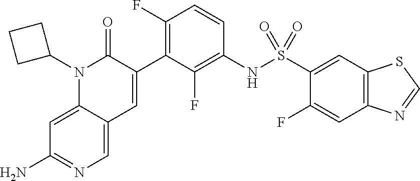 [SMILES: Nc1cc2c(cn1)cc(-c1c(F)ccc(NS(=O)(=O)c3cc4scnc4cc3F)c1F)c(=O)n2C1CCC1] | N-(3-(7-amino-1-cyclobutyl- 2-oxo-1,2-dihydro-1,6- naphthyridin-3-yl)-2,4- difluorophenyl)-5- fluorobenzo[d]thiazole-6- sulfonamide | 557.9 |
| P-0025 | 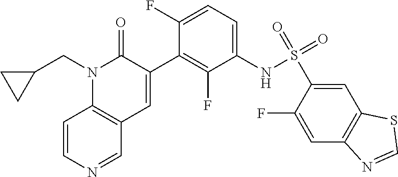 [SMILES: O=c1c(-c2c(F)ccc(NS(=O)(=O)c3cc4scnc4cc3F)c2F)cc2cnccc2n1CC1CC1] | N-(3-(1- (cyclopropylmethyl)-2-oxo- 1,2-dihydro-1,6- naphthyridin-3-yl)-2,4- difluorophenyl)-5- fluorobenzo[d]thiazole-6- sulfonamide | 542.9 |
| P-0026 | 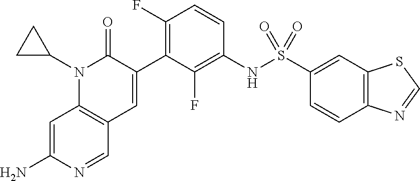 [SMILES: Nc1cc2c(cn1)cc(-c1c(F)ccc(NS(=O)(=O)c3ccc4ncsc4c3)c1F)c(=O)n2C1CC1] | N-(3-(7-amino-1- cyclopropyl-2-oxo-1,2- dihydro-1,6-naphthyridin-3- yl)-2,4- difluorophenyl)benzo[d] thiazole-6-sulfonamide | 525.8 |
| P-0027 | 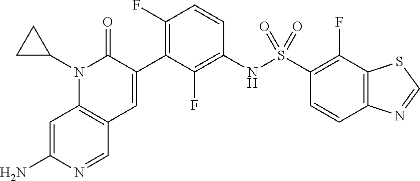 [SMILES: Nc1cc2c(cn1)cc(-c1c(F)ccc(NS(=O)(=O)c3ccc4ncsc4c3F)c1F)c(=O)n2C1CC1] | N-(3-(7-amino-1- cyclopropyl-2-oxo-1,2- dihydro-1,6-naphthyridin-3- yl)-2,4-difluorophenyl)-7- fluorobenzo[d]thiazole-6- sulfonamide | 543.9 |
| P-0028 | 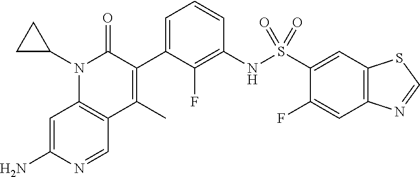 [SMILES: Cc1c(-c2cccc(NS(=O)(=O)c3cc4scnc4cc3F)c2F)c(=O)n(C2CC2)c2cc(N)ncc12] | N-(3-(7-amino-1- cyclopropyl-4-methyl-2- oxo-1,2-dihydro-1,6- naphthyridin-3-yl)-2- fluorophenyl)-5- fluorobenzo[d]thiazole-6- sulfonamide | 539.9 |
| P-0029 | 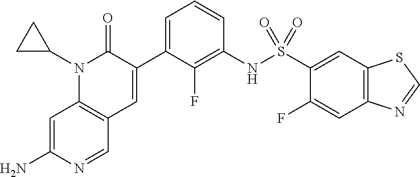 [SMILES: Nc1cc2c(cn1)cc(-c1cccc(NS(=O)(=O)c3cc4scnc4cc3F)c1F)c(=O)n2C1CC1] | N-(3-(7-amino-1- cyclopropyl-2-oxo-1,2- dihydro-1,6-naphthyridin-3- yl)-2-fluorophenyl)-5- fluorobenzo[d]thiazole-6- sulfonamide | 526.4 |
| P-0030 | 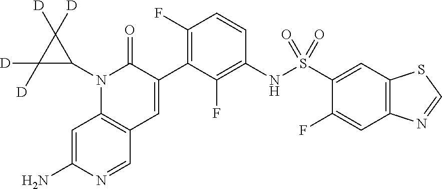 [SMILES: [2H]C1([2H])C(n2c(=O)c(-c3c(F)ccc(NS(=O)(=O)c4cc5scnc5cc4F)c3F)cc3cnc(N)cc32)C1([2H])[2H]] | N-(3-(7-amino-1- (cyclopropyl-2,2,3,3-d4)-2- oxo-1,2-dihydro-1,6- naphthyridin-3-yl)-2,4- difluorophenyl)-5- fluorobenzo[d]thiazole-6- sulfonamide | 548.8 |

### Biological Examples

### Biological Test Methods

### Background

CSK is a tyrosine kinase that regulates the activity of the SRC-family tyrosine kinases, which includes the lymphocyte-specific kinase LCK. LCK is activated following engagement of the T-cell receptor and undergoes autophosphorylation at a tyrosine residue within the active site (Y394). Catalytic activation of LCK is critical for the initiation of signaling downstream of the T-cell receptor and the resulting proliferation of T-cells. CSK phosphorylates a C-terminal tyrosine residue (Y505) on LCK which inhibits LCK catalytic activity, and blocks T-cell proliferation. Modest inhibition of CSK has been demonstrated to induce activation of LCK and increased T-cell proliferation in response to low affinity ligand stimulation of the T-cell receptor (Manz et al., 2015). Therefore development of CSK-specific inhibitors is a potential therapeutic approach to increase the T-cell anti-tumor response. To achieve the desired effect on T-cells, the inhibitor must show activity against CSK but not against LCK. This selectivity can be monitored through in vitro activity assays for both CSK and LCK.

To assess the ability of CSK inhibitors to activate LCK and stimulate signaling downstream of the T-cell receptor, an NFAT reporter assay was used for monitoring of LCK activation in cells through the activation of LCK-dependent signaling through a reporter assay. The Jurkat T-cell cell line was genetically engineered to express a luciferase reporter that is regulated by the NFAT-response element. (Clipstone, N. A., & Crabtree, G. R., Identification of calcineurin as a key signalling enzyme in T-lymphocyte activation, Nature, 357(6380), 695-697, 1992). Stimulation of the T-cell receptor with anti-CD3 antibody increases the expression of the luciferase reporter. To test the ability of CSK inhibitors to stimulate LCK and downstream T-cell signaling, the cells were stimulated with a suboptimal concentration of anti-CD3 ligand, to measure the ability of CSK inhibitors to increase luciferase expression.

### Human CSK Enzymatic Assay

This assay measures the ability of CSK to phosphorylate a biotinylated peptide containing multiple tyrosine residues in vitro. The CSK enzyme was purchased from Thermo Fisher. The biotinylated Poly (Glu4-Tyr) peptide substrate was purchased from Sigma-Aldrich. The CSK enzyme assay was performed using 0.75 ng CSK enzyme and 30 nM Poly (Glu4-Tyr) substrate in the presence of 25 mM Hepes pH 7.5, 5 mM Mg Acetate, 0.1% NP-40, 1 mM DTT, 0.01% BSA, and 100 μM ATP. A 19 μL volume of the above reaction mixture was added to wells of 384-well AlphaPlate (PerkinElmer GA, USA) containing 1 μL of various concentrations of test compound or DMSO vehicle and incubated for 60 minutes at room temperature. 16 wells containing all the components of reaction mixture and 5% DMSO serve as high control. 16 wells containing all the components except ATP of reaction mixture and 5% DMSO served as low control. The enzymatic reaction was stopped by the addition of 5 μL stop/detection mixture containing 25 mM Hepes pH 7.5 100 mM EDTA, and 5 μg/mL streptavidin-coated AlphaScreen donor beads (PerkinElmer GA, USA) and incubated for 20 minutes. A 5 μL mixture containing 25 mM Hepes pH 7.5, 100 mM EDTA, and 5 μg/mL anti-phosphotyrosine antibody PY20-coated AlphaScreen acceptor beads (PerkinElmer GA, USA) was added and the plate was incubated for 1 hour at room temperature. The streptavidin donor beads bind to the biotin moiety on the biotinylated Poly (Glu4-Tyr) substrate and the PY20 antibody-coated acceptor beads bind to tyrosine residues on the substrate that have been directly phosphorylated by CSK. Upon excitation of these beads with laser light at 680 nm, singlet oxygen was produced. This singlet oxygen was rapidly quenched, unless the AlphaScreen PY20 acceptor beads are in close proximity, in which case a proximity signal can be measured at 580 nm. In the presence of catalytic activity, there was a very strong proximity signal. Selective CSK inhibitors affect a decrease in this proximity signal through a decrease in phosphorylation of the Poly (Glu4-Tyr) substrate. The percentage of inhibition at individual concentrations relative to high and low controls was calculated. The data were analyzed by using nonlinear regression to generate IC₅₀ values. Human LCK enzymatic assay

This assay measures the ability of LCK to phosphorylate a biotinylated peptide containing multiple tyrosine residues in vitro. The LCK enzyme was purchased from ProQinase. The biotinylated Poly (Glu4-Tyr) peptide substrate was purchased from Sigma-Aldrich. The LCK enzyme assay was performed using 1 ng LCK enzyme and 30 nM Poly (Glu4-Tyr) substrate in the presence of 25 mM Hepes pH 7.5, 10 mM MnCl₂, 50 mM NaCl, 0.01% Tween-20, 0.5 mM DTT, 0.01% BSA, and 100 μM ATP. A 19 μL volume of the above reaction mixture was added to wells of 384-well AlphaPlate (PerkinElmer GA, USA) containing 1 μL of various concentrations of test compound or DMSO vehicle and incubated for 40 minutes at 25° C. 16 wells containing all the components of reaction mixture and 5% DMSO served as high control. 16 wells containing all the components except ATP of reaction mixture and 5% DMSO served as low control. The enzymatic reaction was stopped by the addition of 5 μL stop/detection mixture containing 25 mM Hepes pH 7.5 100 mM EDTA, and 10 μg/mL streptavidin-coated AlphaScreen donor beads (PerkinElmer GA, USA) and incubated for 20 minutes. A 5 μL mixture containing 25 mM Hepes pH 7.5, 100 mM EDTA, and 10 μg/mL anti-phosphotyrosine antibody PY20-coated AlphaScreen acceptor beads (PerkinElmer GA, USA) was added and the plate was incubated for 1 hour at room temperature. The streptavidin donor beads bind to the biotin moiety on the biotinylated Poly (Glu4-Tyr) substrate and the PY20 antibody-coated acceptor beads bind to tyrosine residues on the substrate that have been directly phosphorylated by LCK. Upon excitation of these beads with laser light at 680 nm, singlet oxygen was produced. This singlet oxygen was rapidly quenched, unless the AlphaScreen PY20 acceptor beads are in close proximity, in which case a proximity signal can be measured at 580 nm. In the presence of catalytic activity, there was a very strong proximity signal. Selective LCK inhibitors affect a decrease in this proximity signal through a decrease in phosphorylation of the Poly (Glu4-Tyr) substrate. The percentage of inhibition at individual concentrations relative to high and low controls was calculated. The data were analyzed by using nonlinear regression to generate IC₅₀ values.

### NFAT Reporter Assay

This assay measures the signaling activity downstream of the T-cell receptor, specifically the ability of the NFAT transcription factor to be translocated to the nucleus and induce expression of a firefly luciferase gene. Jurkat/NFAT-RE cells stably expressing the NFAT reporter construct were purchased from BPS Bioscience. The assay was performed by plating 40,000 Jurkat/NFAT-RE cells in a 50 μL volume of serum-free growth media in a 96-well plate. Following an overnight incubation, the cells were treated with CSK inhibitors and 0.02 μg/mL anti-CD3 antibody OKT3 (BD Bioscience) for 3 hours at 37° C. High control wells were stimulated with DMSO and 5 μg/mL OKT3 to maximally stimulate the NFAT reporter, and low control wells were stimulated with DMSO and 0.02 μg/mL OKT3. Following the 3 hour incubation, the cells were assayed for viability using CellTiter-Fluor™ (Promega), and for luciferase expression using ONE-Glo™ (Promega). The ONE-Glo™ signal was normalized to the CellTiter-Fluor™ signal, and the percentage of NFAT activation relative to high and low controls was calculated. The data were analyzed by using nonlinear regression to generate EC50 values. Selective CSK inhibitors affect an increase in luciferase expression in the presence of low level stimulation of the T-cell receptor.

The following Table 2 provides data indicating biochemical and/or cell inhibitory activity for exemplary compounds as described herein in Table 1. In Table 2 below, activity is provided as follows: +++=0.001 μM<IC₅₀<0.5 μM; ++=0.5 μM<IC₅₀<5 μM, +=5 μM<IC₅₀<100 μM.

TABLE 2

| | | | Jurkat EC50 (uM) |
|---|---|---|---|
| | CSK IC50 (μM) | LCK IC50 (uM) | Cell Assay: Reporter |
| | COFACTOR: | COFACTOR: | Cell Variant: NFAT |
| P # | 100 uM ATP | 100 uM ATP | response element |
| P-0001 | +++ | +++ | +++ |
| P-0002 | +++ | ++ | +++ |
| P-0003 | +++ | + | +++ |
| P-0004 | +++ | + | ++ |
| P-0005 | +++ | + | +++ |
| P-0006 | +++ | + | ++ |
| P-0007 | +++ | + | ++ |
| P-0008 | +++ | + | +++ |
| P-0009 | +++ | + | ++ |
| P-0010 | +++ | + | ++ |
| P-0011 | +++ | + | ++ |
| P-0012 | +++ | ++ | +++ |
| P-0013 | +++ | ++ | +++ |
| P-0014 | +++ | + | ++ |
| P-0015 | ++ | + | ++ |
| P-0016 | +++ | + | +++ |
| P-0017 | +++ | + | +++ |
| P-0018 | +++ | + | +++ |
| P-0019 | +++ | ++ | +++ |
| P-0020 | +++ | ++ | +++ |
| P-0021 | +++ | ++ | +++ |
| P-0022 | +++ | ++ | +++ |
| P-0023 | +++ | ++ | +++ |
| P-0024 | +++ | + | +++ |
| P-0025 | ++ | + | + |
| P-0026 | +++ | + | ++ |
| P-0027 | +++ | | +++ |
| P-0028 | + | + | |
| P-0029 | +++ | + | |
| P-0030 | | | |

All patents and other references cited herein are indicative of the level of skill of those skilled in the art to which the disclosure pertains, and are incorporated by reference in their entireties, including any tables and figures, to the same extent as if each reference had been incorporated by reference in its entirety individually.

One skilled in the art would readily appreciate that the present disclosure is well adapted to obtain the ends and advantages mentioned, as well as those inherent therein. The methods, variances, and compositions described herein as presently representative of the embodiments described herein are exemplary and are not intended as limitations on the scope of the disclosure. Changes therein and other uses will occur to those skilled in the art, which are encompassed within the spirit of the disclosure, are defined by the scope of the claims.

It will be readily apparent to one skilled in the art that varying substitutions and modifications may be made to the present disclosure described herein without departing from the scope and spirit of the disclosure. For example, variations can be made to provide additional compounds of the compounds of this disclosure and/or various methods of administration can be used. Thus, such additional embodiments are within the scope of the present disclosure and the following claims.

The present disclosure illustratively described herein suitably may be practiced in the absence of any element or elements, limitation or limitations which is not specifically described herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically described by the embodiments and optional features, modification and variation of the concepts herein described may be resorted to by those skilled in the art, and that such modifications and variations are considered to be within the scope of this disclosure as defined by the appended claims.

In addition, where features or aspects of the disclosure are described in terms grouping of alternatives, those skilled in the art will recognize that the disclosure is also thereby described in terms of any individual member or subgroup of members of the groups described herein.

Also, unless indicated to the contrary, where various numerical values are provided for embodiments, additional embodiments are described by taking any 2 different values as the endpoints of a range. Such ranges are also within the scope of the present disclosure.

Thus, additional embodiments are within the scope of the disclosure and within the following claims.

What is claimed is:

1. A compound of Formula (I): 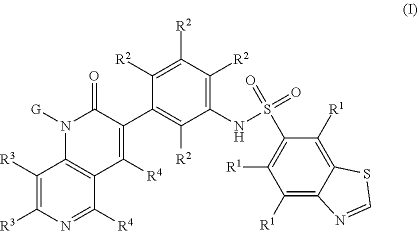
or a pharmaceutically acceptable salt, a tautomer, a stereoisomer, or a deuterated analog thereof, wherein:
G is H, alkyl substituted with 0-4 X³ groups and 0-1 Z¹ group, —[C(X¹)(X²)]₁₋₆-alkoxy substituted with 0-4 X³ groups and 0-1 Z¹ group, —[C(X¹)(X²)]₀₋₆-cycloalkyl substituted with 0-4 X⁴ groups and 0-1 Z¹ groups, or —[C(X¹)(X²)]₀₋₆-heterocycloalkyl substituted with 0-3 X⁴ groups and 0-1 Z¹ group;
each R¹ is independently H, halogen, OH, CN, C₁-C₃alkyl optionally substituted with 1-3 Z² groups, or alkoxy optionally substituted with 1-3 Z² groups;
each R² is independently H, halogen, CN, C₁-C₃alkyl optionally substituted with 1-3 Z² groups, or C₁-C₃alkoxy optionally substituted with 1-3 Z² groups;
each R³ is independently H, CN, halogen, —NHR⁵, alkyl optionally substituted with 1-4 Z² groups, alkoxy optionally substituted with 1-4 Z² groups, alkoxyalkyl optionally substituted with 1-4 Z² groups, cycloalkyl optionally substituted with 1-4 Z³ groups, cycloalkylalkyl optionally substituted with 1-4 Z³, heterocycloalkyl optionally substituted with 1-4 Z³ groups, heterocycloalkylalkyl optionally substituted with 1-4 Z³ groups, heteroaryl optionally substituted with 1-4 Z³ groups, heteroarylalkyl optionally substituted with 1-4 Z³ groups, aryl optionally substituted with 1-4 Z³ groups, or arylalkyl optionally substituted with 1-4 Z³ groups, provided that no more than one R³ is —NHR⁵;
each R⁴ is independently H, —C₁-C₃alkyl, halo, OH, or CN;
R⁵ is H, alkyl optionally substituted with 1-4 Z² groups, alkoxyalkyl optionally substituted with 1-4 Z² groups, cycloalkyl optionally substituted with 1-4 Z³ groups, cycloalkylalkyl optionally substituted with 1-4 Z³ groups, heterocycloalkyl optionally substituted with 1-4 Z³ groups, heterocycloalkylalkyl optionally substituted with 1-4 Z³ groups, heteroaryl optionally substituted with 1-4 Z³ groups, heteroarylalkyl optionally substituted with 1-4 Z³ groups, aryl optionally substituted with 1-4 Z³ groups, or arylalkyl optionally substituted with 1-4 Z³ groups provided that when R⁵ is heterocycloalkyl or heteroaryl, then any heteroatom of R cannot be attached to the nitrogen atom of —NHR⁵;
each X¹ is independently H, halogen or alkyl;
each X² is independently H, halogen or alkyl;
each X³ is independently OH, CN, or halogen;
each X⁴ is independently D, OH, CN, halogen, alkyl optionally substituted with 1-4 Z² groups, or alkoxy optionally substituted with 1-4 Z² groups;
Z¹ is cycloalkyl optionally substituted with 1-4 Z³ groups, cycloalkylalkyl optionally substituted with 1-4 Z³ groups, heterocycloalkyl optionally substituted with 1-4 Z³ groups, heterocycloalkylalkyl optionally substituted with 1-4 Z³ groups, heteroaryl optionally substituted with 1-4 Z³ groups, heteroarylalkyl optionally substituted with 1-4 Z³ groups, aryl optionally substituted with 1-4 Z³ groups, or arylalkyl optionally substituted with 1-4 Z³ groups;
each Z² is independently halogen or OH; and
each Z³ is independently halogen, OH, or alkyl optionally substituted with 1-3 halogens, provided that when Z³ is halogen or OH, then Z³ cannot be attached to any heteroatom from R⁵.

2. The compound according to claim 1 having Formula (II): 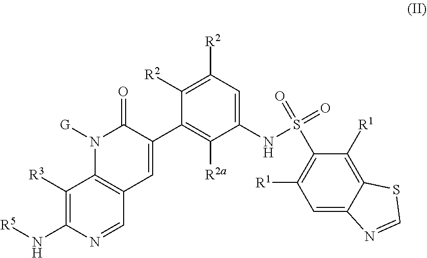
or a pharmaceutically acceptable salt, a tautomer, a stereoisomer, or a deuterated analog thereof, wherein:
G is H, C₁-C₆alkyl substituted with 0-4 X³ groups and 0-1 Z¹ group, —[C(X¹)(X²)]₀₋₄—C₃-C₆cycloalkyl substituted with 0-4 X⁴ groups and 0-1 Z¹ group, —[C(X¹)(X²)]₁₋₄—C₁-C₆alkoxy substituted with 0-3 X³ groups and 0-1 Z¹ group, or —[C(X¹)(X²)]₀₋₄-4-6 membered heterocycloalkyl substituted with 0-3 X⁴ groups and 0-1 Z¹ group;
each R¹ is independently H, halogen, CN, C₁-C₂alkyl optionally substituted with 1-3 Z² groups;
each R² is independently H, halogen, CN, C₁-C₂alkyl optionally substituted with 1-3 Z² groups;
R², is F or Cl;
R³ is H, CN, halogen, C₁-C₃alkyl optionally substituted with 1-3 Z² groups, C₁-C₃alkoxy optionally substituted with 1-3 Z² groups, or C₁-C₃alkoxyC₁-C₃alkyl optionally substituted with 1-3 Z² groups;
R⁵ is H, C₁-C₆alkyl optionally substituted with 1-4 Z² groups, C₁-C₄alkoxyC₁-C₄alkyl optionally substituted with 1-4 Z² groups, C₃-C₆cycloalkyl optionally substituted with 1-4 Z³ groups, —C₁-C₄alkyl-C₃-C₆cycloalkyl optionally substituted with 1-4 Z³ groups, 4-6 membered heterocycloalkyl optionally substituted with 1-4 Z³ groups, —C₁-C₄alkyl-4-6 membered heterocycloalkyl optionally substituted with 1-4 Z³ groups, 5-6 membered heteroaryl optionally substituted with 1-4 Z³ groups, —C₁-C₄alkyl-5-6 membered heteroaryl optionally substituted with 1-4 Z³ groups, phenyl optionally substituted with 1-4 Z³ groups, or —C₁-C₄alkyl-phenyl optionally substituted with 1-4 Z³ groups, provided that when R⁵ is a 4-6 membered heterocycloalkyl or 5-6 membered heteroaryl, then any heteroatom of R cannot be attached to the nitrogen atom of —NHR⁵;
each X¹ is independently H, halogen or methyl;
each X² is independently H, halogen or methyl;
each X³ is independently OH, CN or halogen;
each X⁴ is independently D, OH, CN, halogen, C₁-C₆alkyl optionally substituted with 1-4 Z² groups, or C₁-C₆alkoxy optionally substituted with 1-4 Z² groups;
Z¹ is cycloalkyl optionally substituted with 1-4 Z³ groups, cycloalkylalkyl optionally substituted with 1-4 Z³ groups, heterocycloalkyl optionally substituted with 1-4 Z³ groups, heterocycloalkylalkyl optionally substituted with 1-4 Z³ groups, heteroaryl optionally substituted with 1-4 Z³ groups, or heteroarylalkyl optionally substituted with 1-4 Z³ groups;
each Z² is independently halogen or OH; and
each Z³ is independently halogen, OH, or alkyl optionally substituted with 1-3 halogens, provided that when Z³ is halogen or OH, then Z³ cannot be attached to any heteroatom from R⁵.

3. The compound according to claim 1 having Formula (III): 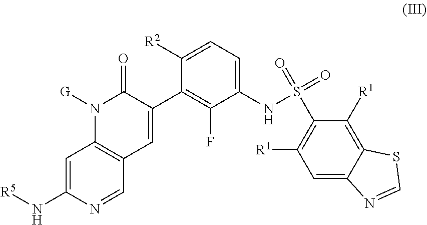
or a pharmaceutically acceptable salt, a tautomer, a stereoisomer, or a deuterated analog thereof, wherein:
G is H, C₁-C₄alkyl optionally substituted with 1-3 X³ groups, —[C(X¹)(X²)]₀₋₂—C₃-C₄cycloalkyl optionally substituted with 1-3 X⁴ groups, or C₁-C₄alkoxy optionally substituted with 1-3 X³ groups;
each R¹ is independently H, F or Cl;
R² is H, F or Cl;
R⁵ is H, C₁-C₆alkyl optionally substituted with 1-3 Z² groups, C₁-C₃alkoxyC₁-C₃alkyl optionally substituted with 1-3 Z² groups, C₃-C₄cycloalkyl optionally substituted with 1-3 Z³ groups, —C₁-C₃alkyl-C₃-C₄cycloalkyl optionally substituted with 1-3 Z³ groups, 4-6 membered heterocycloalkyl optionally substituted with 1-3 Z³ groups, —C₁-C₂alkyl-4-6 membered heterocycloalkyl optionally substituted with 1-3 Z³ groups, 5-6 membered heteroaryl optionally substituted with 1-3 Z³ groups, —C₁-C₂alkyl-5-6 membered heteroaryl optionally substituted with 1-3 Z³ groups, phenyl optionally substituted with 1-3 Z³ groups, or —C₁-C₂alkyl-phenyl optionally substituted with 1-3 Z³ groups, provided that when R⁵ is a 4-6 membered heterocycloalkyl or 5-6 membered heteroaryl, then any heteroatom of R cannot be attached to the nitrogen atom of —NHR⁵;
each X¹ is independently H, F, Cl, or CH₃;
each X² is independently H, F, Cl, or CH₃;
each X³ is independently F, Cl, or OH;
each X⁴ is independently F, Cl, OH or methyl;
each Z² is independently F, Cl or OH; and
each Z³ is independently F, Cl, OH, or C₁-C₃alkyl optionally substituted with 1-3 halogens, provided that when Z³ is F, Cl, or OH, then Z³ cannot be attached to any heteroatom from R⁵.

4. The compound according to claim 1 having one of the following Formulae: 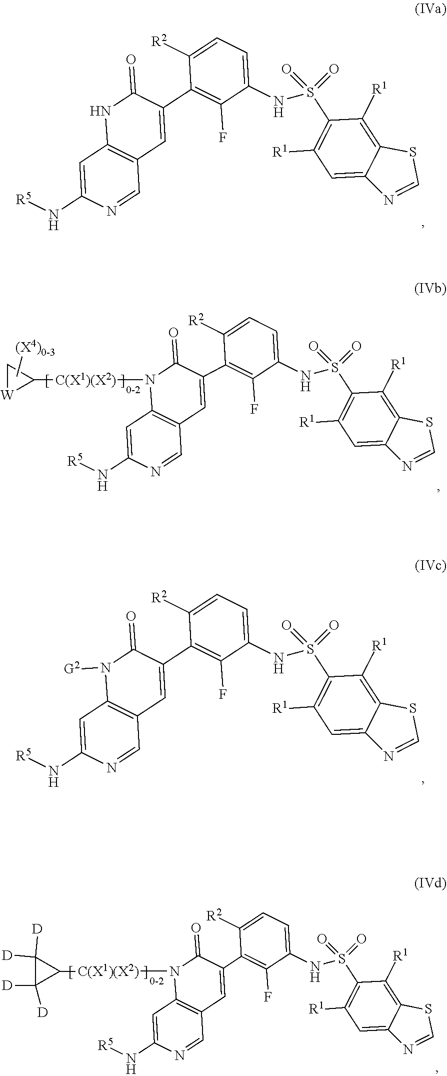
or a pharmaceutically acceptable salt, or a stereoisomer thereof, wherein:
G² is C₁-C₄alkyl optionally substituted with 1-3 X³ groups;
each R¹ is independently H, F or Cl;
R² is H, F or Cl;
R⁵ is H, C₁-C₆alkyl optionally substituted with 1-3 Z² groups, C₁-C₃alkoxyC₁-C₃alkyl optionally substituted with 1-3 Z² groups, C₃-C₄cycloalkyl optionally substituted with 1-3 Z³ groups, —C₁-C₃alkyl-C₃-C₄cycloalkyl optionally substituted with 1-3 Z³ groups, 4-6 membered heterocycloalkyl optionally substituted with 1-3 Z³ groups, —C₁-C₂alkyl-4-6 membered heterocycloalkyl optionally substituted with 1-3 Z³ groups, 5-6 membered heteroaryl optionally substituted with 1-3 Z³ groups, —C₁-C₂alkyl-5-6 membered heteroaryl optionally substituted with 1-3 Z³ groups, phenyl optionally substituted with 1-3 Z³ groups, or —C₁-C₂alkyl-phenyl optionally substituted with 1-3 Z³ groups, provided that when R⁵ is a 4-6 membered heterocycloalkyl or 5-6 membered heteroaryl, then any heteroatom of R cannot be attached to the nitrogen atom of —NHR⁵;
W is —CH₂—, —CH₂CH₂—, or —CH₂CH₂CH₂—;
each X¹ is independently H or F or CH₃;
each X² is independently H, F, or CH₃;
each X³ is independently F, Cl, or OH;
each X⁴ is independently F, Cl, OH or methyl;
each Z² is independently F, Cl or OH; and
each Z³ is independently F, Cl, OH, or C₁-C₃alkyl optionally substituted with 1-3 halogens, provided that when Z³ is F, Cl, or OH, then Z³ cannot be attached to any heteroatom from R⁵.

5. The compound according to claim 4 having Formula (IVa), wherein:
R² is H or F; and
R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R⁵ is a 5-6 membered heteroaryl, then any heteroatom of the 5-6 membered heteroaryl cannot be attached to the nitrogen atom of —NHR⁵.

6. The compound according to claim 4 having Formula (IVb), wherein:
R² is H or F;
R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R⁵ is a 5-6 membered heteroaryl, then any heteroatom of the 5-6 membered heteroaryl cannot be attached to the nitrogen atom of —NHR⁵;
W is —CH₂— or —CH₂CH₂—;
each X² is independently H or F; and
each X⁴ is independently F or methyl.

7. The compound according to claim 4 having Formula (IVc), wherein:
G² is C₁-C₄alkyl optionally substituted with 1-3 F;
R² is H or F; and
R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R is a 5-6 membered heteroaryl, then any heteroatom of the 5-6 membered heteroaryl cannot be attached to the nitrogen atom of —NHR⁵.

8. The compound according to claim 4 having Formula (IVd), wherein:
R² is H or F; and
R⁵ is H, C₁-C₄alkyl optionally substituted with 1-3 Z² groups, C₁-C₃alkoxyC₁-C₃alkyl optionally substituted with 1-3 Z² groups, 5-6 membered heteroaryl optionally substituted with 1-3 Z³ groups, —C₁-C₂alkyl-5-6 membered heteroaryl optionally substituted with 1-3 Z³ groups, provided that when R is a 5-6 membered heteroaryl, then any heteroatom of the 5-6 membered heteroaryl cannot be attached to the nitrogen atom of —NHR⁵.

9. The compound according to claim 1 having one of the following Formulae: 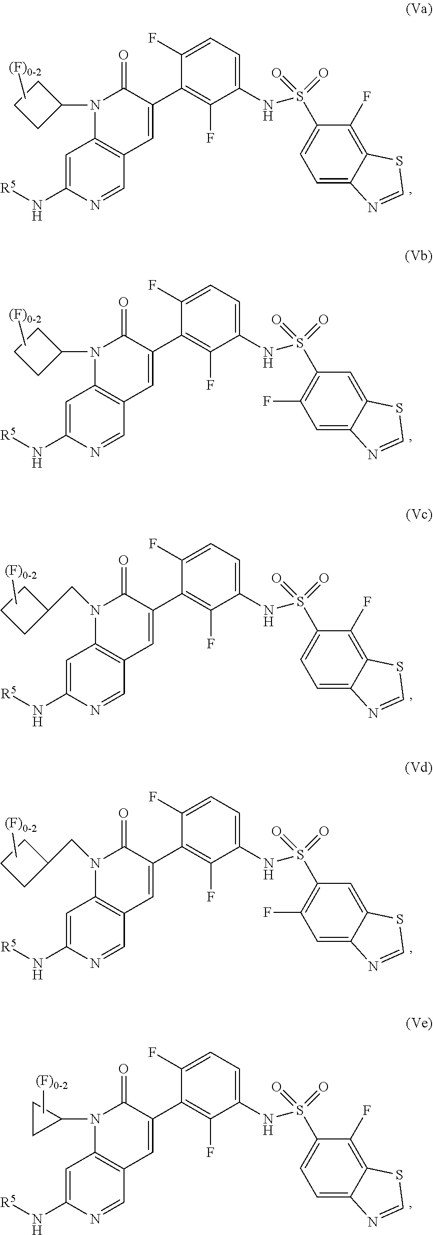 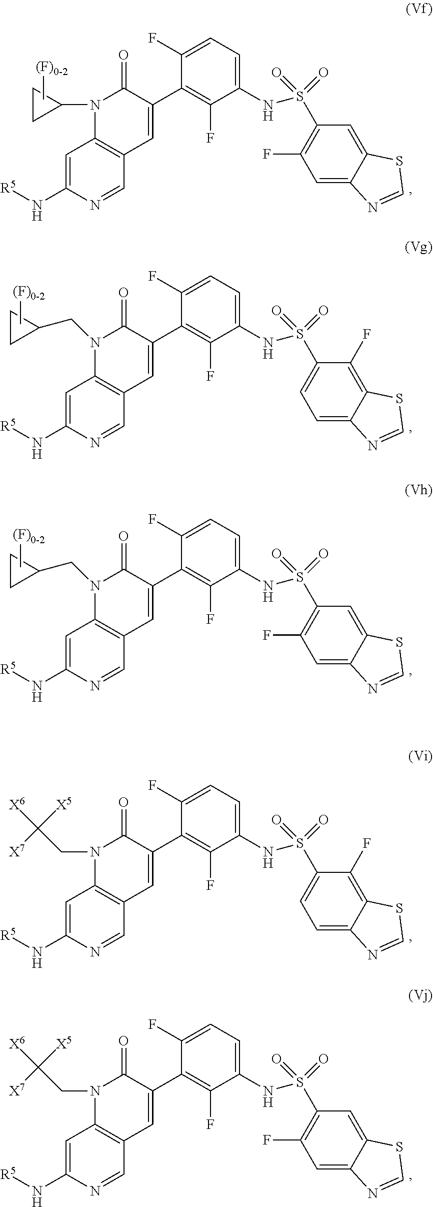 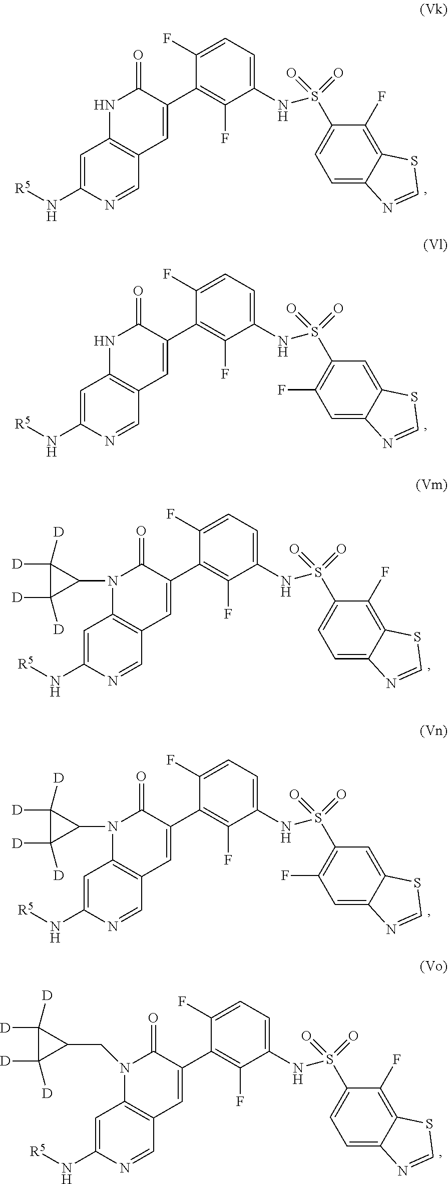 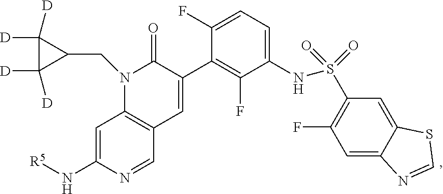
or a pharmaceutically acceptable salt, or a stereoisomer thereof, wherein:
R⁵ is H, C₁-C₆alkyl optionally substituted with 1-3 Z² groups, C₁-C₃alkoxyC₁-C₃alkyl optionally substituted with 1-3 Z² groups, C₃-C₄cycloalkyl optionally substituted with 1-3 Z³ groups, —C₁-C₃alkyl-C₃-C₄cycloalkyl optionally substituted with 1-3 Z³ groups, 4-6 membered heterocycloalkyl optionally substituted with 1-3 Z³ groups, —C₁-C₂alkyl-4-6 membered heterocycloalkyl optionally substituted with 1-3 Z³ groups, 5-6 membered heteroaryl optionally substituted with 1-3 Z³ groups, —C₁-C₂alkyl-5-6 membered heteroaryl optionally substituted with 1-3 Z³ groups, phenyl optionally substituted with 1-3 Z³ groups, or —C₁-C₂alkyl-phenyl optionally substituted with 1-3 Z³ groups, provided that when R⁵ is a 4-6 membered heterocycloalkyl or 5-6 membered heteroaryl, then any heteroatom of R cannot be attached to the nitrogen atom of —NHR⁵;
X⁵ is H or F;
X⁶ is H, F, or CH₃;
X⁷ is H, F, or CH₃;
each Z² is independently F, Cl or OH; and
each Z³ is independently F, Cl, OH, or C₁-C₃alkyl optionally substituted with 1-3 halogens, provided that when Z³ is F, Cl, or OH, then Z³ cannot be attached to any heteroatom from R⁵.

10. The compound according to claim 9 having one of Formulae (Va), (Vb), (Vc), (Vd), or a pharmaceutically acceptable salt, a tautomer, a stereoisomer, or a deuterated analog thereof.

11. The compound according to claim 9 having one of Formulae (Ve), (Vf), (Vg), (Vh), or a pharmaceutically acceptable salt, a tautomer, a stereoisomer, or a deuterated analog thereof.

12. The compound according to claim 9 having one of Formulae (Vi), (Vj), or a pharmaceutically acceptable salt, a tautomer, a stereoisomer, or a deuterated analog thereof.

13. The compound according to claim 9 having one of Formulae (Vk), (Vl), or a pharmaceutically acceptable salt, a tautomer, a stereoisomer, or a deuterated analog thereof.

14. The compound according to any of the preceding claims, wherein R⁵ is H, CH₃, hydroxyethyl, methoxyethyl, or 1-(difluoromethyl)-1H-pyrazolyl, provided that a nitrogen atom of 1-(difluoromethyl)-1H-pyrazolyl cannot be attached to the nitrogen atom of —NHR⁵.

15. A compound selected from Table 1, or a pharmaceutically acceptable salt thereof.

16. A pharmaceutical composition comprising a compound in any one of the preceding claims, and a pharmaceutically acceptable carrier.

17. The pharmaceutical composition of claim 16, further comprising a second pharmaceutical agent.

18. A method for treating a subject with a disease or condition mediated by CSK or T-cell activation, said method comprising administering to the subject an effective amount of a compound in any one of claims 1-15, or a pharmaceutically acceptable salt, deuterated analog, a tautomer or a stereoisomer thereof, or a pharmaceutical composition in any one of claims 16-17.

19. A method for treatment of a disease or condition according to claim 18, wherein there is selective inhibition of CSK over LCK.

20. A method for treatment of a disease or condition according to claim 18 or 19, wherein the disease or condition is a neoplastic disorder, a cancer, an age-related disease, an inflammatory disorder, a cognitive disorder or a neurodegenerative disease.

21. A method for treatment of a disease or condition according to claim 18 or 19, wherein the disease or condition is colorectal cancer, ovarian cancer, breast cancer, lung cancer, liver cancer, prostate cancer, kidney cancer, lymphoma, melanoma, pancreatic cancer, or leiomyosarcoma, e.g. uterine leiomyosarcoma.

22. The method according to any one of claim 18-21, further comprising administering one or more additional therapeutic agents.

23. The method according to claim 22, wherein the one or more additional therapeutic agents is one or more of i) an alkylating agent selected from adozelesin, altretamine, bizelesin, busulfan, carboplatin, carboquone, carmustine, chlorambucil, cisplatin, cyclophosphamide, dacarbazine, estramustine, fotemustine, hepsulfam, ifosfamide, improsulfan, irofulven, lomustine, mechlorethamine, melphalan, oxaliplatin, piposulfan, semustine, streptozocin, temozolomide, thiotepa, and treosulfan; ii) an antibiotic selected from bleomycin, dactinomycin, daunorubicin, doxorubicin, epirubicin, idarubicin, menogaril, mitomycin, mitoxantrone, neocarzinostatin, pentostatin, and plicamycin; iii) an antimetabolite selected from azacitidine, capecitabine, cladribine, clofarabine, cytarabine, decitabine, floxuridine, fludarabine, 5-fluorouracil, ftorafur, gemcitabine, hydroxyurea, mercaptopurine, methotrexate, nelarabine, pemetrexed, raltitrexed, thioguanine, and trimetrexate; iv) an immune checkpoint agent selected from a PD-1 inhibitor, a PD-L1 inhibitor, and an anti-CTLA4 inhibitor; v) a hormone or hormone antagonist selected from enzalutamide, abiraterone, anastrozole, androgens, buserelin, diethylstilbestrol, exemestane, flutamide, fulvestrant, goserelin, idoxifene, letrozole, leuprolide, magestrol, raloxifene, tamoxifen, and toremifene; vi) a taxane selected from DJ-927, docetaxel, TPI 287, paclitaxel and DHA-paclitaxel; vii) a retinoid selected from alitretinoin, bexarotene, fenretinide, isotretinoin, and tretinoin; viii) an alkaloid selected from etoposide, homoharringtonine, teniposide, vinblastine, vincristine, vindesine, and vinorelbine; ix) an antiangiogenic agent selected from AE-941 (GW786034, Neovastat), ABT-510, 2-methoxyestradiol, lenalidomide, and thalidomide; x) a topoisomerase inhibitor selected from amsacrine, edotecarin, exatecan, irinotecan, SN-38 (7-ethyl-10-hydroxy-camptothecin), rubitecan, topotecan, and 9-aminocamptothecin; xi) a kinase inhibitor selected from erlotinib, gefitinib, flavopiridol, imatinib mesylate, lapatinib, sorafenib, sunitinib malate, 7-hydroxystaurosporine, and vatalanib; xii) a targeted signal transduction inhibitor selected from bortezomib, geldanamycin, and rapamycin; xiii) a biological response modifier selected from imiquimod, interferon-α and interleukin-2; xiv) an IDO inhibitor; xv) a chemotherapeutic agent selected from 3-AP (3-amino-2-carboxyaldehyde thiosemicarbazone), altrasentan, aminoglutethimide, anagrelide, asparaginase, bryostatin-1, cilengitide, elesclomol, eribulin mesylate, ixabepilone, lonidamine, masoprocol, mitoguanazone, oblimersen, sulindac, testolactone, tiazofurin, an mTOR inhibitor, a PI3K inhibitor, a Cdk4 inhibitor, an Akt inhibitor, a Hsp90 inhibitor, a farnesyltransferase inhibitor and an aromatase inhibitor (anastrozole letrozole exemestane); xvi) a BRAF inhibitor; xvii) a Mek inhibitor; xviii) c-Kit mutant inhibitor, xix) an EGFR inhibitor, xx) an epigenetic modulator; xxi) other adenosine axis blockade agents selected from CD39, CD38, A2AR and A2BR; or xxii) agonists of TNFA super family member; and xxiii) an anti-ErbB2 mAb.
